SEQUENCE LISTING (1 sequence)

SEQ ID NO: 1 - DNA, 1485 bp, Mus musculus
gagctcgacgcgagccaggagtccggacatctctgcgtccggcgcagcagtcagagctgtccccactgccaccgctcggagccaactccgtctcggacctgggactctggactctaattcgtagctacttctcacctcggggctgcgctgtctccccgctagccttcccgttgtcctccaccgctcaggacgggggtgccacgatgtcccgctgctgcgcagggccccgggcctccctcgacgtgtgaccctagcctggtccccctgcttggctgtccgccctctccttggagacccccggcccggcccccgggggaagaggaagaagacgacgaggccgagggggggatgtccggctccaaaagcgtgagccccccgggctacgctgcacagacagcggcgtcgccagcgccccggggaggcccggagcatcgcgccgcttggggagaagccgattcccgcgccaatggctacccccacgcccccgggggatcaacccgcggctccaccaagagatctgggggagcggtgaccccacaacagcagcagcgcctggccagccgttggcgcggtggcgatgacgacgaagaccctccactaagcggtgatgaccctctggctgggggcttcggcttcagcttccgctctaagtccgcctggcaggagcgcggtggcgacgacggcggtcgcggcagcaggcggcagcggcggggcgcggctggagggggcagcacccgggcgccccctgcgggcggcagcggcagttcggcggcggccgcagcggccgcaggtggcacagaggtgcgcccccgctcggtggagctgggcctggaggagcgtcgaggaaaaggccgagcggccgaggagctggagcccgggactggcatcgtcgaggatggagacgggtcggaggatggaggcagttctgtggcgtcaggctctgggaccggcgcggtgctgtcgttgggcgcctgctgcctggccttgctgcagatattccgctctaagaagttcccgtcggacaaactggagcgtctgtaccagcgctacttcttccacctgaaccagagcagtctcaccatgctcatggccgtgctggtgcttgtgtgcctggtcatgctggctttccacgcggcgcgccccccgctccagatagcctacctggccgtgttggcagctgctgtgggcgtgatccttatcatggccgtgctctgcaaccgtgcagcgttccaccaggaccacatgggcctggcctgctatgcgctcattgcagtggtgctggccgtccaggtagtgggcctgttgctgccacagccacgcagcgcctccgagggcatctggtggaccgtgttcttcatctataccatctacaccctgctgcctgtgcgcatgagggctgcggtgctcagcggggtgcttctgtcggctctccacttggccatctctctgcacaccaactcccaggaccagtttctgctgaaacag

(19) United States
(12) Patent Application Publication
(10) Pub. No.: US 2006/0252774 A1
(43) Pub. Date: Nov. 9, 2006

(54) Regulation of type 5 adenylyl cyclase for treatment of neurodegenerative and cardiac diseases

(76) Inventor: Stephen F. Vatner, New York, NY (US)

Correspondence Address: KLAUBER & JACKSON, 411 HACKENSACK AVENUE, HACKENSACK, NJ, 07601, US

(21) Appl. No.: 10/429,214

(22) Filed: May 2, 2003

Related U.S. Application Data
(60) Provisional application No. 60/377,508, filed on May 2, 2002.
(60) Provisional application No. 60/408,247, filed on Sep. 5, 2002.

(51) Int. Cl. A61K 31/52

(52) U.S. Cl. 514/263.2; 514/263.23

(57) ABSTRACT

The invention concerns pharmaceutical compositions that contain a compound or compounds that can effectively regulate the activity of Type 5 Adenylyl Cyclase and methods for treatment of neurological diseases and disorders, as well as motor function loss therefrom, as well as treatment for cardiac conditions and diseases including conditions characterized by abnormal heart rate.

### CROSS REFERENCE TO RELATED APPLICATIONS

Priority is claimed under 35 USC §119(e) to Provisional Patent Application No. 60/377,508, filed May 2, 2002, and Provisional Patent Application No. 60/408,247, filed Sep. 5, 2002.

### ACKNOWLEDGEMENT OF GOVERNMENT FUNDING

This work was partially funded by the National Institutes of Health through grants HL59729, HL61476, HL67724, HL65183, HL65182, HL69020, HL59139, AG14121, and HL33107, and also by the American Heart Association grants 9940187N, 9950673N and 0020323U and therefore the United States Government may have certain rights in the invention.

### FIELD OF THE INVENTION

The invention relates to the use of Type 5 Adenylyl Cyclase (hereinafter “Type 5 AC” or “AC5”) inhibitors to treat and prevent hypertension and other cardiac diseases or ailments characterized by abnormal heart rate, as well as to the treatment and prevention of acute and chronic motor dysfunction associated with common neurodegenerative disorders such as Parkinson's disease, essential tremor, motor dysfunction after various neuronal infections and/or cerebrovascular accidents including stroke, cerebral trauma, and aneurysms, as well as other neuronal diseases, such as Huntington's disease, torsion dystonia, myoclonus and drug induced movements by modulating the “cyclic AMP (“cAMP”) signaling in the striatum.

### BACKGROUND OF THE INVENTION

Cyclic AMP is a major second messenger that converts an extracellular signal to an intracellular signal. Neurotransmitters and hormones bind to the cell surface G protein-coupled receptors, leading to the activation of Gsalpha protein by promoting the exchange of GDP for GTP. Activated Gsalpha, or the GTP-bound form of Gsalpha, binds to adenylyl cyclase (“AC”) and enhances the rate of catalytic activity of this enzyme, i.e., the conversion of ATP to cAMP. Due to the endogenous hydrolysis activity of Gsalpha, the GTP-bound form of Gsalpha eventually returns to the GDP-bound form of Gsalpha and dissociates from AC. Cyclic AMP, an intracellular second messenger, activates protein kinase A (PKA) by dissociating its regulatory subunit from the catalytic subunit. The catalytic subunit of PKA now initiates an enzymatic cascade of phosphorylation reactions within the cell, for example, various enzymes involved in myocyte contraction in the heart, such as troponin, phospholamban, those involved in glucose metabolism in the liver, such as glycogen phosphorylase, and those involved in neuronal function. By phosphorylating uniquely differentiated proteins in each cell type, cAMP signaling and thus PKA regulate the unique function of each organ. Cyclic AMP is eventually degraded to AMP by phosphodiesterase. PKA is inactivated by re-association of the catalytic subunit with the regulatory subunit⁷. Phosphorylated proteins are dephosphorylated by phosphatases, thereby pulling the protein conformation back into an inactive form.

It is known that the AC isoforms are diverse in both tissue distribution and biochemical properties. The AC isoform originally isolated from the brain (designated as type I) is calmodulin-sensitive and is expressed only in the brain⁸. Subsequently, additional AC isoforms (AC2 through AC9) were isolated, bringing the total number of known isoforms to nine^{9-12 13-15 16,17}. All AC isoforms share the same membrane topology, i.e., a tandem repetition of a six-transmembrane domain and a large cytoplasmic domain. The amino acid sequence within the membrane domain is not conserved among these isoforms; however, that of the cytoplasmic domain is relatively well-conserved and is considered to be the catalytic domain. Interestingly, a group of isoforms shows higher amino acid sequence homology with each other than with other isoforms. Subsequent biochemical studies also revealed that certain isoforms share not only a similar amino acid sequence, but also display similar biochemical properties. Based upon amino acid sequence homology, biochemical properties, and tissue distribution, the nine isoforms can be subdivided into at least five subgroups. Importantly, the diversity in their biochemical properties, in particular, regulation by calmodulin and Gbeta/gamma subunits, and in their tissue distribution may explain the conflicting findings of earlier studies in which membrane preparations from a variety of different tissues were used for AC assays.

The finding of diversity in the AC isoforms and in their regulation during the past decade has expanded our understanding of this classic intracellular signaling pathway. One of the questions yet remaining is the specific role of each isoform in a given cell type and organ function. A particular AC isoform must play a specific role in regulating the physiological function of a given organ. Unfortunately, previous in vitro experimental approaches were unable to address this issue. For this reason, as described in detail below, inventors have developed a transgenic mouse model lacking the expression of AC5. Secondly, advantage is taken of the diversity of AC isoforms, within the brain in a new pharmacotherapy. In particular, inventors have focused on AC5, which is a dominant isoform in the striatum³⁴, but the physiological significance of this stiatum-specific localization has remained unknown. The striatum is known to coordinate motor function of the body via receiving dopamingeric input that activates the striatal AC, thereby the regulation of AC5, a major effector enzyme of the dopamine receptor in the striatum, may regulate motor function. Unlike classic pharmacotherapy that targets the adrenergic receptor or dopamine receptor, for example, to regulate neuronal activity, such as L-dopa therapy, targeting the dominant AC isoform may have several advantages. Unlike dopamine receptors that are widely expressed, AC5 expression is limited to the striatum within the brain, and not readily detectable in other parts of the brain and the heart. The dopamine or beta-adrenergic receptors may undergo desensitization or massive down regulation under pathological conditions, leading to the loss of the receptor on the neuronal cell surface, while changes in AC5 expression is slow and the magnitude of changes is small, as inventors have demonstrated^{41,42}. Accordingly, AC5 specific regulators might replace beta-adrenergic receptor blockers, which are most commonly used to treat hypertension but are contraindicated in asthma patients because tracheal beta-adrenergic receptors are also blocked. In addition, AC5 specific regulators might replace dopamine receptor regulators, which are commonly used to treat Parkinson's disease, the most common motor function disease among the elderly. However, a major problem is the development of tolerance after chronic usage of this medication, which is most likely due to the changes at the level of dopamine receptors. The underlying hypothesis is that the AC5 isoform bears a distinct physiological role in the striatum to regulate motor function, either stimulatory or inhibitory. Disruption of such an AC isoform leads to impaired organ function that is directly or indirectly related to the unique property of this AC isoform. Therefore, establishing a method to regulate this isoform in a specific manner will lead to the regulation of its specific function in the striatum.

AC1 and AC8 form the neuronal subgroup, which are expressed only in the brain. They are both stimulated by calmodulin. Sequence homology between these two isoforms is the least conserved among all the subgroups. Gbeta/gamma subunits inhibit the catalytic activity of AC1 and AC8. There are at least three splice variants of the AC8 isoform that differ in calmodulin sensitivity¹⁸; in contrast, two potential splice variants of the AC1 isoform do not show any functional differences¹⁹. AC2, AC4, and AC7 form the ubiquitous subgroup, which are expressed in multiple tissues, including the heart. They are insensitive to calmodulin. In contrast to the neuronal isoforms, the ubiquitous isoforms are stimulated by Gbeta/gamma subunits. AC4 and AC7 can be detected in most tissues although one variant of AC7 is expressed only in the retina²⁰. The ubiquitous isoforms are potently stimulated by protein kinase C^{21,22}, although other isoforms can also be stimulated by this kinase as well²³. AC2 seems to be the major isoform in the lung; this isoform is expressed in airway smooth muscle cells²⁴ and vascular endothelial cells from a variety of tissues²⁵. Since this isoform responds to a variety of signals emanating from different receptors and G proteins²⁶, including chemotactic²⁷ and growth factor receptors, its output represents an integrated response to multiple extracellular stimuli, placing it at a key position to modulate airway resistance²⁴. Originally isolated from olfactory tissue, AC3 is calmodulin-sensitive⁹. Although its expression is highest in olfactory tissues, its mRNA can also be detected in other tissues such as the atria and brown fat²⁸.

AC5 and AC6 are the most closely related isoforms within the mammalian AC family. Although these isoforms were cloned from several tissue sources including the heart^{12,13}, the liver¹⁴ and neuronal cells¹⁵, these represent the major AC isoforms in the heart. In addition, AC5 is the dominant isoform in the striatum of the brain²⁹. AC9 is the newest member of the mammalian AC family. This isoform, isolated from a pituitary tumor cell line, shows a unique interaction with calcineurin^{30,31}, a Ca-sensitive serine-threonine phosphatase widely expressed in mammalian cells. This isoform can be inhibited by FK543, a calcineurin inhibitor, suggesting that the activity of this isoform is maintained though phosphorylation.

Thus, there is significant heterogeneity in the distribution and biochemical properties of the various AC isoforms. It is also apparent that a single tissue or cell expresses multiple AC isoforms: no cell type has been found thus far that expresses only one isoform of AC. Each tissue and cell type, however, likely possess a unique “mixture” of AC isoforms. The heterogeneity among AC isoforms distinguishes this enzyme family from the other components of the beta-adrenergic signaling pathway, specifically the beta-adrenergic receptor itself and Gsalpha, which lack this diversity either in the number of isoforms expressed or in their pattern of tissue-specific expression.

A number of neurotransmitters and neuromodulators in the brain are mediated though G protein-coupled receptors, including those of the classical neurotransmitters, dopamine, serotonin, and adrenaline. All the AC isoforms are subject to the regulation of G proteins and thus AC is a crucial molecule in modulating the physiological responses of this broadly expressed neurotransmission and neuromodulation system. The diversity of the AC family members may allow each isoform to function in a different signal transduction pathway of neurotransmitters, neuromodulators or neurotrophic factors. This is particularly important for the neuronal system, unlike the heart, in which a single neuron may receive stimulating and/or sequential multiple inputs from other neurons in a fraction of a second. Further, the mode of this input may differ from one region from the other in the brain. The coincidence detector of AC renders neurons capable of detecting simultaneous stimulation of two or more neurotransmitters. This neuronal integration of multiple signals may be determined by the biochemical characteristics of the AC that is expressed by the particular neuron. Because of the complexity and extensive involvement of AC in neuronal information processing, AC has been implicated in biological functions from synaptic plasticity and circadian rhythms³².

The distribution of the AC isoforms within the brain is heterogeneous, suggesting that each isoform is involved in a distinct aspect of neuronal signaling³³. The hippocampus is rich in AC1 and since this isoform is activated by Ca-calmodulin, it has been speculated that it plays a role in long-term potentiation mediated by the glutamate receptor. The olfactory bulb is rich in AC3. AC5 is most dominant in the striatum, implicating its involvement in motor regulation³⁴. AC5 is located mostly in medium-sized striatal neuronal cells expressing D1 dopaminergic receptors in the basal ganglia, and accordingly has been implicated in signal detection to dopaminergic function. In contrast, most AC6 is present in most neurons and is co-localized with various neurotransmitters systems, AC6 might be in regulation of the classical neuronal signal integration on the brain³⁵. However, the role of these isoforms in neuronal function in vivo is poorly understood. A key question that remains unanswered is what the specific role of an AC isoform is in neuronal function and cAMP signal whose expression is limited to a specific brain region.

AC5 and AC6 isoforms are abundantly expressed in the brain and the heart. The expression of these isoforms are regulated both developmentally⁴⁰ and pathophysiologically^{41,42}. However, under pathological conditions, the magnitude of change of AC was much smaller than that of the beta-adrenergic receptor. In contrast to the studies in the heart, changes in the expression of these isoforms, both developmentally and pathophysiologically, in the striatum have not been extensively examined.

Various molecules that may regulate the activity of AC5 have been examined. It has been demonstrated that AC5 is susceptible to dual regulation through phosphorylation: inhibition by protein kinase A⁴³ and stimulation by protein kinase C^{44,45}. Importantly, kinase-mediated regulation occurs only for a subset of AC isoforms, i.e., isoform-specifically. Molecules that can regulate AC in an isoform-specific manner are not limited to kinase and other classic AC regulators. Inventors have found that caveolin, a major protein component of caveolae, is another regulator of AC. Using peptides whose sequence was derived from caveolin subtypes, AC catalytic activity can be regulated in a caveolin-subtype and an AC isoform-specific manner⁴⁶. This study led to identification of the isoform-specific cross talk between AC and caveolin, as well as subcellular localization of AC and its related molecules in caveolae^{42,46-54}.

These findings led to a search for isoform-specific pharmacological regulators of AC. A result from such efforts is the finding of a forskolin derivative (NKH477) that stimulates AC5 with enhanced selectivity⁵⁸. This study was significant in that it was demonstrated, for the first time, that a classic pharmacological AC regulator (forskolin), which regulates AC non-selectively, can be developed into an AC5 isoform-specific regulator.

Indeed, inventors have found an even more selective stimulator (new forskolin derivative) of AC5 as well as an inhibitor of AC5^{58,59}. In these studies, inventors used the findings from crystallographic studies that have elucidated the binding mechanism of forskolin and P-site inhibitors to AC.

These findings lead to an investigation of neurodegenerative disorders, such as Parkinson's disease, which is characterized by akinesia, tremor, and rigidity that result largely from progressive degeneration of dopaminergic neurons in the Substantia Nigra (“SN”). Although the mechanisms responsible for neuronal degeneration in the Parkinson's disease remain unclear, numerous studies have tried to recapitulate the phenotype of this disease in experimental animals. A classic example is the toxin 1-methyl-4-phenyl-1,2,3,6-tetrahydropyridine (MPTP)-induced animal model, which induces Parkinson's disease-like pathology and behavioral symptoms¹⁰³. MPTP is metabolized by the enzyme monoamine oxidase (MAO) to 1-methyl-4-phenylpyridinium, which is transported selectively into dopaminergic terminals, and then concentrates in the mitochondria, where it induces oxidative stress and impairment of complex I activity¹⁰⁴. More recent studies have suggested that Parkinson's disease may be caused by a gene mutation(s). As an example, consider that mutations in the “parkin gene”, which is involved in ubiquitin-mediated proteolysis, are shown to cause autosomal recessive juvenile Parkinson's disease¹⁰⁵. Transgenic studies have demonstrated that, alteration in the amount of expression of several signaling molecules mimic the phenotype of Parkinson's disease. This result has been observed in several experiments, including alpha-synuclein overexpression mice¹⁰⁶, D1 dopaminergic receptor knockout mice⁶⁷ and retinoid receptor knockout mice¹⁰⁷.

More specifically, the retinoid receptor knockout reduced the expression of dopamine (D1 and D2) receptors in the striatum and blunted the locomotor function. The striatum receives neuronal input from the cortex, feed backing its signal to thalamus and cortex via the SN to coordinate the extra-pyramidal signal and regulate motor function. Neuronal activity of the striatum is modulated by dopaminergic neurons that are derived from the SN. Dopamine receptors in the striatum, both D1 and D2 subtypes, are coupled to AC via G protein to modulate cAMP signal within the striatum. Degeneration of these nigro-striatal neurons, i.e., the loss of dopaminergic input to the striatum, is a hallmark of pathological changes seen in Parkinson's disease, which is represented by kynesia, tremor, and the loss of coordinated movements. Parkinson's disease is common among the elderly; its incidence is second only to Alzheimer's disease. The nature of this neuronal dysfunction, however, is poorly understood. Previous studies demonstrated that alteration of the dopaminergic signal, such as the depletion of dopamine, produces a motor dysfunction that mimics Parkinson's disease, which can be restored by the administration of dopamine. A common problem in the current pharmacotherapy to regulate intracellular cAMP signal, such as beta-adrenergic receptor blockade or dopamine antagonist therapy, arises from the non-organ-selective effect of these drugs on multiple organs. For example, the dopamine-1 receptor is expressed abundantly in the brain, but also in the kidneys, while the beta-andrenergic receptor is expressed ubiquitously. Although dopamine-1 and dompamine-2 selective agonists and beta-adrenergic receptor blockers have been developed to avoid this problem, these receptors are expressed in various tissues including the gastrointestinal tract. In particular, major problems in treating Parkinson's disease by L-dopa, the most commonly prescribed drug for Parkinson's is the development of dyskinesia, meaning a “wearing-off phenomenon and hallucinations.” These effects are believed to be in part caused by changes at the level of the receptor as well as the effects of the drug on cellular proteins other than the dopamine receptor, such as the production of free radicals, which are toxic to neuronal cells. Similarly, beta 1-adrenergic receptor is expressed in the trachea and such drugs may cause airway obstruction and are contraindicated in patients having asthma.

Pharmacological inhibition of AC5 may enable us to regulate striatal function and cardiac function in an organ-specific manner because the expression of AC5 is dominant in the striatum and the heart, but scarce in the other organs. The tissue-selective expression of AC5 is more prominent in large animals such as humans than in small animals such as rodents, as demonstrated in a previous study⁴¹. Such drugs may be effective in pharmacotherapy for high blood pressure, arrhythmia or locomotor dysfunction, which is commonly seen in various neurodegenerative disorders such as Parkinson's disease, Huntington's disease, essential tremor, torsion dystonia, myoclonus, or Wilson's disease. In addition, motor dysfunction after cerebrovascular accidents is a preeminent problem in advanced countries, where hyperlipidemia, diabetes, or obesity are common. Targeting the striatum independent from other peripheral organs may be achieved by altering the hydrophobicity of AC5 inhibitors and thus regulating the passage of the blood brain barrier, as inventors have demonstrated with a forskolin analog in a previous paper¹²⁷.

The above work addressed the role of cAMP, a second messenger molecule that regulates various neuronal functions. Neovasculization is commonly seen in regions of the brain after stroke and helps neurons to survive in an ischemic condition. The process of neovasculization may be accelerated in the presence of cAMP as well as stem cells from various origins (most easily obtained from peripheral blood cells). Thus stem cells and cAMP may synergistically promote neovasculization. The possibility exists for using an adenovirus harboring type 5 adenylyl cyclase that possesses a high enzymatic activity to produce cAMP in conjunction with stem cells and thereby provide long term treatment for neuronal death caused by ischemic stress or chronic neurodegenerative disorders.

### SUMMARY OF THE INVENTION

The invention provides compositions and methods for treating neurodegenerative and cardiac diseases by administering to a patient an effective amount an AC5 regulator.

In a first aspect, the invention provides compositions and methods for the treatment of hypertension and other cardiac diseases and ailments characterized by abnormal heart rate by administering to a patient a pharmacologically effective amount of an AC5 regulator. Suitable AC5 regulators within the purview of the invention include, but are not limited to, forskolin derivatives according the formulae disclosed herein in a pharmaceutically acceptable form.

In a second aspect, the invention provides compositions and methods for the treatment of motor dysfunction associated with neurodegenerative disorders and diseases including Parkinson's disease by administering to a patient a pharmacologically effective amount of an AC5 regulator. Suitable AC5 regulators within the purview of the invention include, but are not limited to, forskolin derivatives according the formulae disclosed herein.

In a third aspect, the invention provides compositions and methods for treating motor dysfunction occurring after neuronal infections such as West Nile virus, or after cerebrovascular trauma and accidents such as stroke and aneurysm by administering to a patient a pharmacologically effective amount of an AC5 regulator. Suitable AC5 regulators within the purview of the invention include, but are not limited to, forskolin derivatives according the formulae disclosed herein.

In a fourth aspect, the invention provides compositions and methods for the treatment of neuronal diseases such as Hutington's disease, torsion dystonia, myoclonus, and drug-induced movements by administering to a patient a pharmacologically effective amount of an AC5 regulator. Suitable AC5 regulators within the purview of the invention include, but are not limited to, forskolin derivatives according the formulae disclosed herein.

In a fifth aspect, the invention provides a method for screening pharmaceutically active treatments for all of the aforementioned diseases, disorders, and ailments by administering said treatment to a mouse having the genotype described in Sequence 1.

In a sixth aspect, the invention is a recombinant vector comprising the isolated nucleotide Sequence 1.

In a seventh aspect, the invention is a gent targeting vector comprising the nucleotide Sequence 1 operatively associated with a selection marker for neomycin resistance and transfected into a host cell thereby altering the adenylyl cyclase expression in the host.

These and other aspects of the invention will be made more clear by reference to the following Figures and Detailed Description of the Preferred Embodiments.

### BRIEF DESCRIPTION OF THE FIGURES

FIG. 1A: Targeted disruption of the AC5 gene in a non-conditional manner: (a) Partial structure of the AC5 gene (Wild type), targeting vector construct[,] (Targeting vector), and (b) resultant mutated allele are shown. The position of the pgk-neo cassette (neo) and 5′-probe (EcoRI-HindIII; 400-bp) are indicated. K, KpnI; E, EcoRI; X, XhoI, A, ApaI; P, PstI; BS, BssHII, S, SpeI, H, HindIII, RV, EcoRV; N, NcoI; B, BamHI.

FIG. 1B: Southern blot analysis of genomic DNA from the offspring of F1-heterozygote intercross using 5′-probe.

FIG. 1C: Comparison of mRNA expression in AC5 and non-disrupted wild type (“WT”) mouse.

FIG. 1D: RNase protection assay showing no compensatory increase in AC3, AC4, AC6, AC7, or AC9 upon disruption of AC5 gene in mice.

FIG. 2A: Comparison of steady state AC activity measured as maximal capacity of cAMP production in heart membranes of AC5KO and WT in the presence of ISO (100 μM ISO+100 μM GTP), GTPγS (100 μM) or forskolin (100 μM).

FIG. 2B: Comparison of inhibition of ISO-stimulated AC activity in WT and AC5KO in the presence of carbachol (10 μM) (a muscrarinic agonist).

FIG. 3A: Comparison of basal and ISO stimualted cardiac function in WT and AC5KO. B: Parasympathetic control of Heart Rate in WT and AC5 KO.

FIGS. 4A, 4B, and 4C: Comparison of Heart Rate in WT and AC5KO under muscarinic stimulation with atropine.

FIG. 5: Impaired rotor-rod performance in AC5KO mice[.] : Three month old male were used (n=10 to 20). Data represent mean±SEM of five trials.

FIG. 6: cAMP production in primary cultured striatal neurons: Forskolin (50 micro M) and dopamine (100 micro M) stimulated cAMP accumulation was measured (Mean±SEM, n=4).

FIG. 7: AC activity in AC5KO[.:]. The steady state AC activity in the striatum (basal, GTPgammaS, and forskolin) and the cerebellum (forskolin), and the cortex (forskolin) were determined. Stimulation was performed at the level of G protein(100 micro M GTPgammaS) and AC (100 micro M forskolin).

FIG. 8: Rearings (left), locomotor (middle) and pole test (right) in AC5KO, hetero and WT.[:] Male mice of 3 month old were used from each group (n=10-20)*p<0.01.

FIG. 9: Effects of db-cAMP on rotor-rod test. Tests were performed before (pre), 30 minutes after (post), and 48 hours after (next) the injection of db-cAMP to WT, hetero and AC5KO (n=4-7).

FIG. 10: Effect of NKY80 and HI30435 on cAMP accumulation in H9C2 cells.[:] H9C2 cells were incubated in the presence of 10 micro M forskolin and isoproterenol and NKY80 or HI30435 (10 micro M), followed by cAMP accumulation assays (n=4-5, p<0.01). Note that NKY80 did not, but HI30435 inhibited cAMP accumulation in cells.

FIG. 11. Concentration-response curves of HI30435 (A) and 3′-AMP (B) on various AC isoforms: AC catalytic activity was measured in the presence of 5 mM MgCl2 with Gsalpha/GTPgammaS/forskolin (50 micro M). The relative inhibitory activity versus control (% control activity) for various AC isoforms are shown. Data are means±S.D. of quadruplicate determinations.

FIG. 12: Effect of HI30435 on AC activity in various brain regions and organs. Membrane preparations from the cerebellum, the cortex, and the striatum (12A) of WT and AC5KO, as well as (12B) those from the heart and lungs of WT were compared.

FIG. 13: Targeted disruption of the AC5 gene in conditional manner[.]: Partial structure of the AC5 gene (Wild-type), targeting vector construct (Targeting vector), recombinant allele in targeted ES cell and first exon deleted mutant. The position of the pgk-neo cassette (neo), hsv-tk cassette (tk), and IoxP site (IoxP) are indicated. After homologous recombination in ES cells, the IoxP-flanked marker cassette was removed by transient expression of Cre-recombinase, and the resulting ES cells were used to generate conditional KO mouse. K, KpnI; E, EcoRI; X, XhoI, A, ApaI; P, PstI; BH, BssHII, S, SpeI, H, HindIII, RV, EcoRV; N, NcoI; B, BamHI.

FIG. 14: Comparison of the neuropeptide mRNA expression between AC5-KNO and WT. Three representative neuropeptides for the dopaminergic signaling in the striatum, for example, dynorphin and substance P for D1 and enkephalin for D2, were quantified by nuclease protection assays.

FIG. 15: Effect of forskolin, 6[3-(dimethylamino)propionyl]-14,15-dihydroforskolin, and NKH477 on adenyl cyclase [isforms.] isoforms: Forskolin derivatives dose response effect (A-C). High five cell membranes overexpressing type II (A), type III (B), and type V (C) adenyl cyclases. Adenyl cyclase activity was measured in the presence of 5 mM of MgCl₂ with various concentrations of forskolin (closed circles), 6[3-(dimethylamino)propionyl]-14,15-dihydroforskolin (D, open triangles) or NKH477 (open circles). Means±SEM from 4 independent experiments are shown. *, p<0.01 differences from the value with forskolin.

FIG. 16: RNase protection assays of types 2, 3, 4, 6, 7, 9 AC and 28S rRNA in the hearts of 4-6 pairs of WT (+/+) and AC5KO (−/−). cRNA of the 28S rRNA was used as an internal control. Types 1 and 8 AC were hardly detectable (data not shown). Representative autoradiograph of each AC isoform and 28S rRNA is shown in he top panel. Quantitation of relative intensities of each AC isoform to 28 S rRNA are shown in the bargraph, NS=not significant.

FIG. 17: Adrenergic, muscarinic, and Ca²⁺-mediated regulation of cardiac AC activity. A, AC activity in vitro. The steady state AC activity was determined as the maximal cAMP production over 15 min. Stimulation was performed at the level of the β-AR (ISO), G-protein (GTPγS) and AC catalytic unit with forskolin (Fsk). *P<0.01, n=5. B, Effects of carbachol on AC activity. AC was preactivated by ISO. AC activity was then determined in the absence and presence of carbachol. Carbachol at 10 μmol/L produced its maximal inhibitory effect. *P<0.01, n−5. C, To investigate the inhibition of AC activity by Ca²⁺, we examined cAMP production in membranes from the hearts of WT and AC5KO at increasing Ca²⁺ concentrations in the presence of ISO. ISO-stimulated AC activity was inhibited more in WT than in AC5KO. The value of 0.08 μmol/L.

FIG. 18: Response of cardiac function to ISO or ACh in vivo. LVEF in response to β-AR stimulation with ISO, 0.04 μg/kg/min i.v., was significantly attenuated in AC5KO. Acetylcholine (ACh) superimposed in ISO reduced LVEF in WT, but its inhibition was attenuated in AC5KO. Absolute values of the responses to ISO and of ACh in the presence of ISO are plotted at the top. Bars representing the absolute changes in responses of LVEF to ISO and then to ACh in the presence of ISO are plotted on the bottom. *P<0.05, n=11.

FIG. 19: Muscarinic regulation of cardiac function in vivo. A, Effects of atropine. Baseline HR was significantly elevated in conscious AC5KO *P<0.01, n=14-15. Administration of atropine increased HR dose-dependently in conscious WT, but the elevation by atropine was impaired in AC5KO. B, Effect of ACh. Administration of ACh attenuated HR dose-dependently in conscious WT, but the inhibition by ACh at the dose of 0.01 mg/kg was impaired in AC5KO. *P<0.01, n=5. C, Baroreflex regulation of HR. Baroreflex slowing of HR in response to phenylephrine induced increase in arterial pressure is shown by the plot of systolic arterial pressure (SAP) vs. the inverse of heart rate, i.e., the R-R interval (msec). The depressed slope indicates that reflex parasympathetic bradycardia was impaired in AC5KO.

FIG. 20: Western blot analysis for protein expression of Gsα, Giα, Gqα, Gβ, Gγ, β-adrenergic receptor kinase (βARK), as well as α₁-AR and muscarinic receptor type 2 (mAChR) in WT and AC5KO. There were no differences in any of these proteins in AC5KO.

FIG. 21: A, Carbachol-activated K⁺ current in atrial myocytes isolated from WT and AC5KO. The cells are held at −40 mV and carbachol was applied as indicated at the bar above each trace. B, Mean carbachol-induced current density. Peak outward K⁺ currents were normalized to cell capacitance to yield current density (pA/pF). Data are means±SEM of WT (n=27) and AC5KO (n=16) cells.

FIG. 22: A, Effects of ISO (1 μmol/L) on I_{Ca} in WT and AC5KO myoctyes. Traces show currents recorded from a holding potential of −50 mV to indicated potentials in control before (a) and after application of ISO (1 μmol/L) (b). Peak I_{Ca} were normalized to the cell capacitance to give current densities (pA/pF) and were plotted as a function of voltage (c). B, Concentration-dependent effects of ISO on I_{Ca} measured in myocytes dialyzed with EGTA or BAPTA, and on Ba²⁺ currents with EGTA. The relative increase of peak current amplitude was plotted against ISO concentration. The solid lines were best fit to one-to-one binding model. Data are from 8-30 myocytes.

FIG. 23. Comparison of cardiac hypertrophy after aortic banding in WT and AC5KO. Transverse aortic banding or sham operation was applied to either WT or AC5KO. (A) LV weight (LVW; mg)/tibial length (TL; mm) was determined at 1 and 3 weeks. LVW/TL of sham-operated animals was obtained at 1 and 3 weeks, and the data were combined. The degree of cardiac hypertrophy increased progressively at 1 and 3 weeks, but was similar in both WT and AC5KO (n=6 for 1 week and n=8-10 for 3 weeks) (B) Cardiac myocyte cross-sectional area was determined at 3 weeks. There was also no significant difference in myocyte cross-sectional area between WT and AC5KO. (n=4-5 each for sham and banded), NS, not significant.

FIG. 24. Changes in LV function after banding in WT and AC5KO Echocardiographic measurements of LV function [LV end-diastolic diameter (EDD) (A) and LV ejection fraction (EF) (B)] were performed in WT and AC5KO after 1 and 3 weeks of banding. The data were compared with those from sham (S) operated controls at 1 and 3 weeks. LVEDD was significantly increased (A) and LVEF was significantly decreased (B) after 3 weeks of banding in WT (n=8) but not in AC5KO (n=10) while both determinations were unchanged between in sham-operated and 1 week-banded mice in WT (n=6) and AC5KO (n=6). *P<0.05, NS, not significant.

FIG. 25. Comparison of TUNEL staining after banding between WT and AC5KO (A) DAPI staining and TUNEL staining of the LV myocardium at 3 weeks after aortic bnding in WT and AC5KO. A white arrow indicates a TUNEL-positive myocyte nucleus. Bar=50 μm. (B) TUNEL-positive myocytes in LV myocardium were counted in WT and AC5KO and expressed as % of myocytes. The number of TUNEL-positive myocytes was significantly smaller in AC5KO than in WT after either 1 or 3 weeks of banding (b=6 each). *P<0.05.

FIG. 26. Western blotting and RNase protection assay of Bcl-2 after banding in WT and AC5KO (A) Expression of Bcl-2 after 3 weeks of aortic banding was compared between WT and AC5KO. Protein expression of Bcl-2 was determined by Western blot analysis. Although Bcl-2 was hardly detectable in the sham groups (data not shown), it was detected after banding in both WT and AC5KO; however, expression of Bcl-2 in AC5KO was greater than that in WT. The Bcl-2 expression level in WT after banding was taken as 100% in each experiment. *P<0.05, n=9-10. Representative immunoblots of Bcl-2 after separation by 4-20% SDS-PAGE are shown in the top panel. (B) The mRNA level of Bcl-2 after 3 weeks of banding was compared between WT and AC5KO. mRNA of Bcl-2 was determined by RNase protection assays. Relative intensity of Bcl-2 to 18S rRNA was shown in the bar graph. Representative autoradiograph of Bcl-2 and 18S rRNA was shown in the top panel, n=5. NS; not significant.

FIG. 27. Motor dysfunction in ACD-^{-/-} mice. Rotarod test (A)—Each mouse was placed on a 3.5-cm diameter rod covered with rubber to evaluate rotarod performance (14). Mice were left for 1 minute on the rod for habituation. The rod rotated gradually increasing from 4 to 40 rpm over the course of 5 min and the time that mice could stay on an accelerating rotarod without failing was recorded. Five trials were conducted for each individual 10-25 min apart within the dark phase of the light/dark cycle. Mice that stayed on the rotarod for >300 sec were considered complete responders, and their latencies were recorded as 300 sec. *p<0.05, relative to ⁺/₊; ⁺/₋, n=17-20. Activity tests (B and C)—The tests were performed as previously described (14, 15). Mice were put in the darkened testing room 60 minutes for habituation before testing. Locomotor activity activity (B) was assessed using an activity monitor equipped with photocell beams (Columbus Instruments, Ohio, USA). The number of photobeam interruptions in each perpendicular axis was recorded and totaled for 5 min. The behavior of the mice was recorded simultaneously by video camera for later analysis, and their rearing actions were counted (C). *p<0.05, relative to ⁺/₊, n=15-20. Pole test (D)—In order to evaluate bradykinesia, pole test was performed (16). In brief, mice were placed head upward on the top of a rough-surfaced pole (8 mm in diameter and 50 cm in height) that was wrapped with gauze to prevent slipping. The time until it turned completely downward (open bars, Tturn) and the time until it climbed down to the floor (closed bars, TLA) were measured *p<0.01, relative to ⁺/₊, n=14-17. Means±SEM are shown. Homozygous to (ACD^{-/-}) (closed circles, ⁻/₋), heterozygous (shaded circles, ⁺/₋), and wild type (WT) (open circles, ⁺/₊).

FIG. 28: AC activity and its mRNA expression in various brain regions. AC catalytic activity was compared as previously described (15) using the membrane preparations from the striatum, cortex and cerebellum A). AC assays were conducted in the presence of 50 μM forskolin. Expression of AC5 mRNA (AC5) was quantitated by RNase protection assays with 28S rRNA as loading standard (28S RRNA) (B). A representative result is shown. D1 or D2 receptor agonist-stimulated AC activity was also examined (C). AC assays were conducted in the presence of 1 μM SKF 38392 and 10 μM quinpirole (QPL) in a reaction buffer containing 10 μM GTP. Each AC catalytic activity was compared to that in the presence of 10 μM GTP alone. Open bars, WT (⁺/₊); closed bars, AC5KO (⁻/₋). Means±SEM are shown. *p<0.05, **p<0.01, relative to ⁺/₊, n=4.

FIG. 29. Expression of receptors, Gs, neurotransmitters and AC isoforms in the striatum Dopaminergic receptor subtype expression was quantitated by radioligand binding assays as previously described using ³H-SCH23390 (left, for D1) and ³H-spiperone (right, for D2) in striatal membrane preparations (A). Relative changes in the Bmax values are shown in percent. Note that preliminary experiments demonstrated the Kd values of D1 and D2 were similar to those previously reported. Open bars, WT (⁺/₊); closed bars, AC5KO (⁻/₋). Means±SEM are shown. *p<0.01, n=5. Expression of Gs protein in the striatal membranes was determined by immunoblotting (B). Upper, a representative immunoblotting of Gs protein (long, the long Gs form; short, the short Gs form). Lower, comparison of the amount of both Gs forms (Gs short and Gs long) between WT and AC5KO. Open bars, WT (⁺/₊); closed bars, AC5KO (⁻/₋). Means±SEM are shown. *p<0.01, n=4-6. Expression of dynorphin (Dyn), substance-P (Sub-P), and enkephalin (Enk) mRNA were compared by RNase protection assays (C). 28S rRNA was used for standardization. Relative values were compared between WT (open bars, ⁺/₊) and AC5KO (closed bars, ⁻/₋). Means±SEM are shown. *p<0.05, n=4-6. Comparative levels of each AC isoform mRNA expression (AC1-AC9) were determined by RNase protection assays (D). All AC isoforms except AC4 and AC8 could be detectable. 28S rRNA was used for standardization. Open bars, WT (⁺/₊); closed bars, AC5KO (⁻/₋). Means±SEM are shown. *p<0.05, n=4-7.

FIG. 32. Effect of dopaminergic agonists on motor functions Effects of SKF38393 on locomotor activity (A), rotarod test (B), and pole test (C) were compared. SKF38393, a D1 dopaminergic agonist, was administered to WT (⁺/₊) and AC5KO (⁻/₋) subcutaneously (open bars, vehicle; shaded bars, 25 mg/kg; closed bars, 50 mg/kg). Means±SEM are shown. * p<0.05, **p<0.01, compared with vehicle, n=7-14 in (A) and (B), and 9-14 in (C). In rotarod test, the best performance out of five trials in each individual was evaluated. Effects of cabergoline on locomotor activity (D), rotarod performance (E), and pole test (F) were compared. Cabergoline, a D2 dopaminergic agonist, was administered to WT (⁺/₊) and AC5KO (⁻/₋) subcutaneously (open bars, vehicle; shaded bars, 0.2 mg/kg; closed bars, 1.0 mg/kg). After injection, mice were placed in a holding cage until testing. Means±SEM are shown. *p<0.05, **p<0.01, compared with vehicle, n=7-15 in (D), 8-14 in (E), and 8-12 in (F).

### DETAILED DESCRIPTION OF THE PREFERRED EMBODIMENTS

Definitions

Alkenyl, alkinyl, and alkynyl.

Alkenyl refers to a trivalent straight chain or branched chain unsaturated aliphatic radical.

Alkinyl or Alkynyl refers to a straight or branched chain aliphatic radical that includes at least two carbons joined by a triple bond.

If no number of carbons is specified alkenyl and alkinyl each refer to radicals having from 2-12 carbon atoms.

Alkyl refers to saturated aliphatic groups including straight-chain, branched-chain and cyclic groups having the number of carbon atoms specified, or if no number is specified, having up to 12 carbon atoms. The term “cycloalkyl” as used herein refers to a mono-, bi-, or tricyclic aliphatic ring having 3 to 14 carbon atoms and preferably 3 to 7 carbon atoms.

Carbocyclic ring structure, C₃₋₁₆ carbocyclic mono, bicyclic or tricyclic ring structure, or the like are each intended to mean stable ring structures having only carbon atoms as ring atoms wherein the ring structure is a substituted or unsubstituted member selected from the group consisting of: a stable monocyclic ring which is aromatic ring (“aryl”) having six ring atoms; a stable monocyclic non-aromatic ring having from 3 to 7 ring atoms in the ring; a stable bicyclic ring structure having a total of from 7 to 12 ring atoms in the two rings wherein the bicyclic ring structure is selected from the group consisting of: ring structures in which both of the rings are aromatic; ring structures in which one of the rings is aromatic; and ring structures in which both of the rings are non-aromatic; and a stable tricyclic ring structure having a total of from 10 to 16 atoms in the three rings, wherein the tricyclic ring structure is selected from the group consisting of: ring structures in which three of the rings are aromatic; ring structures in which two of the rings are aromatic; and ring structures in which three of the rings are non-aromatic. In each case, the non-aromatic rings when present in the monocyclic, bicyclic or tricyclic ring structure may independently be saturated, partially saturated, or fully saturated. Examples of such carbocyclic ring structures include, but are not limited to, cyclopropyl, cyclobutyl, cyclopentyl, cyclohexyl, adamantyl, cyclooctyl, bicyclooctane, bicyclononane, bicyclodecane (decalin), bicyclooctane, fluorenyl, phenyl, naphthyl, indanyl, adamantyl, or tetrahydronaphthyl (tetralin). Moreover, the ring structures described herein may be attached to one or more indicated pendant groups via any carbon atom which results in a stable structure. The term “substituted” as used in conjunction with carbocyclic ring structures means that hydrogen atoms attached to the ring carbon atoms of ring structures described herein may be substituted by one or more of the substituents indicated for that structure if such substitution(s) would result in a stable compound.

Aryl, which is included with the term “carbocyclic ring structure”, refers to an unsubstituted or substituted aromatic ring, substituted with one, two or three substituents selected from lower alkoxy, lower alkyl, lower alkylamino, hydroxy, halogen, cyano, hydroxyl, mercapto, nitro, thioalkoxy, carboxaldehyde, carboxyl, carboalkoxy and carboxamide, including, but not limited to, carbocyclic aryl, heterocyclic aryl, and biaryl groups and the like, all of which may be optionally substituted. Preferred aryl groups include phenyl, halophenyl, loweralkylphenyl, napthyl, biphenyl, phenanthrenyl and naphthacenyl.

Arylalkyl, which is included with the term “carbocyclic aryl”, refers to one, two, or three aryl groups having the number of carbon atoms designated, appended to an alkyl group having the number of carbon atoms designated. Suitable arylalkyl groups include, but are not limited to, benzyl, picolyl, naphthylmethyl, phenethyl, benzyhydryl, trityl, and the like, all of which may be optionally substituted.

As used herein, the term “heterocyclic ring” or “heterocyclic ring system” is intended to mean a substituted or unsubstituted member selected from the group consisting of stable monocyclic ring having from 5-7 members in the ring itself and having from 1 to 4 hetero ring atoms selected from the group consisting of N, O and S; a stable bicyclic ring structure having a total of from 7 to 12 atoms in the two rings wherein at least one of the two rings has from 1 to 4 hetero atoms selected from N, O and S, including bicyclic ring structures wherein any of the described stable monocyclic heterocyclic rings is fused to a hexane or benzene ring; and a stable tricyclic heterocyclic ring structure having a total of from 10 to 16 atoms in the three rings wherein at least one of the three rings has from 1 to 4 hetero atoms selected from the group consisting of N, O and S. Any nitrogen and sulfur atoms present in a heterocyclic ring of such a heterocyclic ring structure may be oxidized. Unless indicated otherwise the terms “heterocyclic ring” or “heterocyclic ring system” include aromatic rings, as well as non-aromatic rings which can be saturated, partially saturated or fully saturated non-aromatic rings. Also, unless indicated otherwise the term “heterocyclic ring system” includes ring structures wherein all of the rings contain at least one hetero atom as well as structures having less than all of the rings in the ring structure containing at least one hetero atom, for example bicyclic ring structures wherein one ring is a benzene ring and one of the rings has one or more hetero atoms are included within the term “heterocyclic ring systems” as well as bicyclic ring structures wherein each of the two rings has at least one hetero atom. Moreover, the ring structures described herein may be attached to one or more indicated pendant groups via any hetero atom or carbon atom which results in a stable structure. Further, the term “substituted” means that one or more of the hydrogen atoms on the ring carbon atom(s) or nitrogen atom(s) of the each of the rings in the ring structures described herein may be replaced by one or more of the indicated substituents if such replacement(s) would result in a stable compound. Nitrogen atoms in a ring structure may be quaternized, but such compounds are specifically indicated or are included within the term “a pharmaceutically acceptable salt” for a particular compound. When the total number of O and S atoms in a single heterocyclic ring is greater than 1, it is preferred that such atoms not be adjacent to one another. Preferably, there are no more than one O or S ring atoms in the same ring of a given heterocyclic ring structure.

Examples of monocyclic and bicyclic heterocyclic ring systems, in alphabetical order, are acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolinyl, indolizinyl, indolyl, 3H-indolyl, isobenzofuranyl, isochromanyl, isoindazolyl, isoindolinyl, isoindolyl, isoquinolinyl (benzimidazolyl), isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyroazolidinyl, pyrazolinyl, pyrazolyl, pyridazinyl, pryidooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl and xanthenyl. Preferred heterocyclic ring structures include, but are not limited to, pyridinyl, furanyl, thienyl, pyrrolyl, pyrazolyl, pyrrolidinyl, imidazolyl, indolyl, benzimidazolyl, 1H-indazolyl, oxazolinyl, or isatinoyl. Also included are fused ring and spiro compounds containing, for example, the above heterocyclic ring structures.

As used herein the term “aromatic heterocyclic ring system” has essentially the same definition as for the monocyclic and bicyclic ring systems except that at least one ring of the ring system is an aromatic heterocyclic ring or the bicyclic ring has an aromatic or non-aromatic heterocyclic ring fused to an aromatic carbocyclic ring structure.

Alkylene Chain refers to straight or branched chain unsaturated divalent radical consisting solely of carbon and hydrogen atoms containing no unsaturation and having from one to six carbon atoms, e.g., methylene, ethylene, propylene, butylenes, and the like. The term “methylene” refers to —CH₂—. The term “Bu” refers to “butyl” or —CH₂CH₂CH₂CH₂—; the term “Ph” refers to “phenyl”; the term “Me” refers to “methyl” or —CH₃; the term “Et” refers to “ethyl” or —CH₂CH₃; the term “Bu(t)” or “t-Bu” refers to “tert-butyl” or —C(CH₃)₄.

Biological property refers to an in vivo effector or antigenic function or activity that is directly or indirectly performed by a compound of this invention that is often shown by in vitro assays. Effector functions include receptor or ligand binding, any enzyme activity or enzyme modulatory activity, any carrier binding activity, any hormonal activity, any activity in promoting or inhibiting adhesion of cells to an extracellular matrix or cell surface molecules, or any structural role. Antigenic functions include possession of an epitope or antigenic site that is capable of reacting with antibodies raised against it.

Diabetes refers to a life-long disease for which there is no cure. There are several types of diabetes including insulin-dependent diabetes (Type I); noninsulin-dependent diabetes (Type II); and gestational diabetes. For all types of diabetes the metabolism of carbohydrates, proteins and fats are altered.

Dopamine 1 refers to the Dopamine receptor responsible for aspects of motor function and cardiovascular function and increases cAMP.

Dopamine 2 refers to the Dopamine receptor responsible for aspects of motor function and cardiovascular function and decreases cAMP.

Dyskinesia refers to abnormal involuntary movement.

Dystonia refers to a disabling but rarely fatal disorder characterized by involuntary muscle contractions which force certain parts of the body into abnormal, sometimes painful, movement or postures.

Essential Tremor refers to a neurological disorder involving shaking that is typically elicited with activity and purposeful movement. Tremors may be occasional, temporary or intermittent and may affect any part of the body.

Myoclonus refers to a rapid spasm of the soft palate, facial muscles and the diaghram, palatal myoclonus or the legs, nocturnal myoclonus. Palatal myoclonus is usually caused by lesions on the brain/nerve pathways whereas some nocturnal myoclonus may be caused by peripheral nerve disease and other cases are caused by unknown factors. Noncturnal myoclonus is also known as Restless Leg Syndrome.

Enkephalin refers to any peptide that has the sequence Tyrosine-Glycine-Glycine-Phenalyine-Xaa.

Forskolin refers to a plant derived substance from the Coleus forskohlii that activation of the enzyme, adenylate cyclase, which in turn increase cyclic adenosine monophosphate (cAMP) in cells.

Halo or halogen refer to Cl, Br, F or I substituents. The term “haloalkyl”, and the like, refer to an aliphatic carbon radicals having at least one hydrogen atom replaced by a Cl, Br, F or I atom, including mixtures of different halo atoms, e.g. trihaloalkyl includes trifluoromethyl and the like as preferred radicals.

Hyperlipidemia. There are 6 types of hyperlipidemia which are differentiated by the type(s) of lipids that are elevated in the blood. Some of the types may be due to a primary disorder such as familial combined hyperlipidemia and some are due to secondary causes. Secondary causes of hyperlipidemia are related to risk factors such as diseases such as diabetes, hypothyroidism, or Cushing's syndrome, diet such as high dietery fat intake, obesity, or excessive alcohol use, or brought on as a side effect of certain pharmacologic compounds.

Hypertension refers to a condition wherein the systolic pressure is consistently over 140 mm Hg, or the diastolic blood pressure is consistently over 90 mm Hg and may be caused by a variety of factors including water volume in the body, salt content of the body, condition of the kidneys, nervous system or blood vessels and hormone levels in the body. The condition of hypertension may also be induced by pharmacological compounds including but not limited to: corticosteriods and other hormones, including estrogens and birth control pills, cyclosporine, and nasal decongestants.

Inhibitor includes but is not limited to, any suitable molecule, compound, protein or fragment thereof, nucleic acid, formulation or substance that can regulate AC5 activity in such a way that AC5 activity is decreased. The inhibitor can include, but is not limited to, the specifically identified ribose-substituted P-site ligands such THFA 9-(tetrahydro-2-furyl) adenine and CPA 9-(cyclopentyl) adenine or 2-amino-7-(2-furanyl)-7,8-dihydro-5(6H)-quinazoline (NKY80).

Left Ventricular Ejection Fraction is an indicator of left ventricular systolic function and is calculated either by echocardiograph or radionuclide ventriculography by estimating the Conceptually, an estimation is made of the volume of blood in the ventricle at the end of diastole and the volume of blood remaining in the ventricle at the end of systole. LVEF is the fraction of blood ejected in systole and is calculated as (Volume at end of diastole−Volume at end of systole )/(Volume of blood at end of diastole).

Mammal refers to any animal classified as a mammal, including humans, domestic and farm animals, and zoo, sports, and pet companion animals, and other domesticated animal such as, but not limited to, cattle, sheep, ferrets, swine, horses, poultry, rabbits, goats, dogs, cats, and the like.

Motor Function refers to the ability of a mammal to control both voluntary and involuntary movement.

Neurodegenerative Disorders refers to a broad class of disorders that present as neurodysfunction, especially the impairment of motor function, including but not limited to: Cerebral palsy, Atrophy, Cerebrovascular ferrocalcinosis, motor neuron disease, and peroneal muscular atrophy.

Parenteral refers to introduction of the polypeptide by intravenous, intraarterial, intraperitoneal, intramuscular, intraventricular, intracranial, subcutaneous, subdermal, transvaginal, oral, nasal or rectal routes.

Patient refers to a mammal, preferably a human, in need of treatment for a condition, disorder or disease.

Pharmaceutically acceptable salts include salts of compounds derived from the combination of a compound and an organic or inorganic acid. These compounds are useful in both free base and salt form. In practice, the use of the salt form amounts to use of the base form; both acid and base addition salts are within the scope of the present invention.

Pharmaceutically acceptable acid addition salt refers to salts retaining the biological effectiveness and properties of the free bases and which are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicyclic acid and the like.

Pharmaceutically acceptable base addition salts include those derived from inorganic bases such as sodium, potassium, lithium, ammonium, calcium, magnesium, iron, zinc, copper, manganese, aluminum salts and the like. Particularly preferred are the ammonium, potassium, sodium, calcium and magnesium salts. Salts derived from pharmaceutically acceptable organic nontoxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, polyamine resins and the like. Particularly preferred organic nontoxic bases are isopropylamine, diethylamine, ethanolamine, trimethamine, dicyclohexylamine, choline, and caffeine.

Prodrug refers to a pharmacologically inactive derivative of a parent drug molecule that requires biotransformation, either spontaneous or enzymatic, within the organism to release the active drug. Prodrugs are variations or derivatives of the compounds of this invention which have groups cleavable under metabolic conditions. Prodrugs become the compounds of the invention which are pharmaceutically active in vivo, when they undergo solvolysis under physiological conditions or undergo enzymatic degradation.

Striatum refers to part of the basal ganglia of the brain and is responsible for motor function.

Stimulator includes but is not limited to, any suitable molecule, compound, protein or fragment thereof, nucleic acid, formulation or substance that can regulate AC5 activity in such a way that AC5 activity is increased. The stimulator can include, but is not limited to, the specifically identified forskolin and its derivatives, divalent cations, peptides and enzymes.

Substance P refers to a neuropeptide secreted upon D1 receptor stimulation in the striatum.

Therapeutically Effective Dose refers to the dose that produces the effects for which it is administered.

Treat, Treatment refers to both therapeutic treatment and prophylactic or preventative measures, wherein the object is to prevent or slow down (lessen) an undesired physiological condition, disorder or disease or to obtain beneficial or desired clinical results. For purposes of this invention, beneficial or desired clinical results include, but are not limited to, alleviation of symptoms; diminishment of extent of condition, disorder or disease; stabilization (i.e. not worsening) a state or condition, disorder or disease; delay or slowing of a condition, disorder, or disease progression; amelioration of the condition, disorder or disease state; remission (whether partial or total), whether detectable or undetectable; or enhancement or improvement of a condition, disorder or disease. Treatment includes eliciting a cellular response that is clinically significant, without excessive side effects. Treatment also includes prolonging survival as compared to expected survival without treatment

Wilson's Disease refers to a rare genetic disorder which prevents the body from properly excreting copper; as a result, the liver and nervous systems are damaged and patients experience a wide array of neurological or psychiatric problems. The disease may mimic Parkinson's Disease.

### PREFERRED EMBODIMENTS

The invention provides compounds capable of regulating the activity of Type 5 Adenolyl Cylase (“AC5”). The invention further provides pharmaceutical compositions containing effective amounts of at least one AC5-regulating compound and also provides methods for the use of these compopsitions in the treatment of cardiac and neurological diseases. In order to evaluate compounds suitable to be utilized in compositions and methods of the invention, a computer-assisted drug design program was used to examine over 200 newly synthesized forskolin derivatives. Forskolin, like digitalis, is a natural plant extract, which has been used in traditional medicine. Forskolin directly activates AC to increase the concentration of intracellular cAMP. This mechanism for activation is now explained as follows. Forskolin binds to the catalytic core at the opposite end of the same ventral cleft that contains the active site, and activates the enzyme by gluing together the two cytoplasmic domains in the core (C₁ and C₂) using a combination of hydrophobic and hydrogen bond interactions. As predicted by a recent crystallographic study, there is a relatively large open space between the C6/C7 positions of forskolin and its binding site within AC. It had been hypothesized that a forskolin derivative modified in these positions might have altered isoform-selectivity without disrupting their activity; this is consistent with the findings.

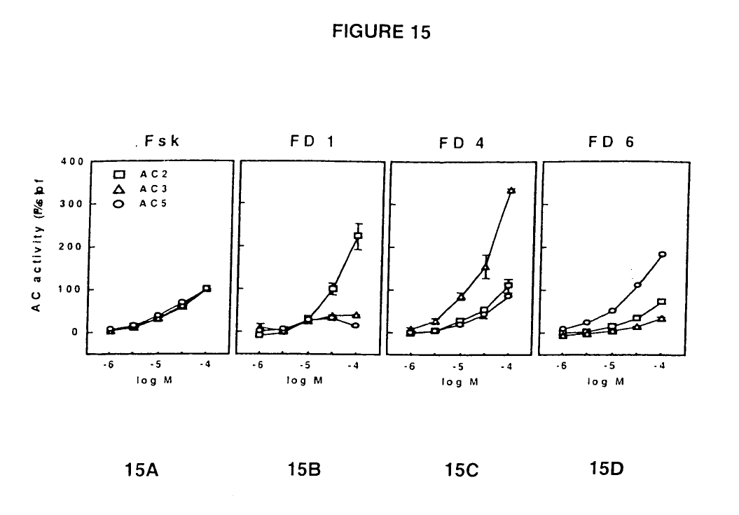

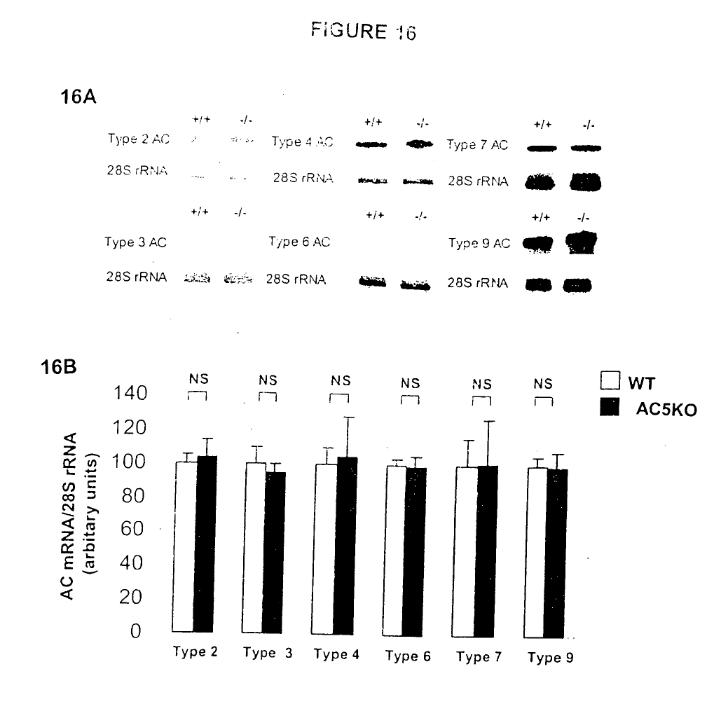

It has been previously reported that 6-[3-(dimethylaminopropionyl forskolin (NKH477) had enhanced stimulation of AC5 while the potency of stimulating other isoforms (AC2 and AC3) remained similar. It should be noted that NKH477! is now used to stimulate cardiac AC in patients with congestive heart failure in some countries. Several other forskolin derivatives, in which a positively charged group, such as 3-(dimethylaminopropionyl group, was attached to the position of C6 or C7, show a similar enhancement in AC5-selectivity. Thus, modification of the C6 or the C7 positions with a positively charged residue results in enhanced AC5-selectivity without losing potency for other AC isoforms. 14,15-Dihydroforskolin has a weak stimulatory effect on AC, but shows a small enhancement in AC5 selectivity. Further modifications, namely, placement of a 3-(dimethylaminopropionyl group at the C6 position of 14,15-dihydroforskolin yields a forskolin derivative, 6-3(dimethylaminopropionyl]-14,15-dihydroforskolin) that possesses a further enhancement in selectivity for AC5. As shown in FIG. 15, the relative potency of stimulation of this derivative versus forskolin was 66% for AC2, 31% for AC3 and 139% for AC5.

Classic inhibitors of AC include adenosine analogs or P-site inhibitors, and MDL12330A, a non-nucleic acid inhibitor. However, not much was known about the isoform selectivity of these inhibitors. Classic P-site inhibitors with phosphate at the 3′ position such as 2′-d-3′-AMP and 3′-AMP potently inhibited AC catalytic activity. 2′-d-3′-AMP potently inhibited AC5 and AC3 while to a lesser degree AC2; the selectivity ratio was 27 between AC5 and AC2. The IC₅₀ values for each isoform were calculated to be 0.82 micro M for AC5, 2.8 micro M for AC3 and 22.4 micro M for AC2. In contrast, ribose-substituted P-site inhibitors, such as THFA and CPA, potently inhibited AC5 while they inhibited AC2 and AC3 only to a modest degree in the presence of Gsalpha/GTPgammaS/forskolin. The IC₅₀ value was calculated as 2.2 micro M for AC5, 101 micro M for AC3 and 285 micro M for AC2. It was previously noted that AC2 was less sensitive to THFA than the other isoforms, giving a selectivity ratio of 1.8 when compared between AC6 and AC2. Inventors found that the selectivity ratio was even greater (130) between AC5 and AC2.

The above data demonstrated that ribose-substituted P-site ligands such as THFA (9-(tetrahydro-2-furyl)adenine) and CPA 9-(cyclopentyl)adenine selectively inhibited AC5. First, inventors looked for the pharmacophore within THFA that is essential for the inhibition of AC5. Inventors found that the presence of an intact adenine structure at the C2 position was important. The intact adenine structure at the C6 position may also be essential for inhibiting AC catalytic activity because a crystallographic study has already shown that the N1 and amino group at the C6 position of the adenine ring bind to AC via hydrogen bonding. Accordingly, inventors screened 850 thousand compounds that are commercially available using a pharmacophore screening algorithm, and selected 682 compounds that have the pharmacophore (═C²H—N¹═C⁶(NH₂)—) in their structure. Inventors then examined 32 representative compounds and identified 2-amino-7-(2-furanyl)-7,8-dihydro-5(6H)-quinazolinone (NKY80). NKY80 showed a similar AC5 selectivity to THFA in inhibiting AC catalytic activity with a selectivity ratio of 210 between AC5 and AC2. The IC₅₀ values were calculated to be 8.3 micro M for AC5, 132 micro M for AC3 and 1.7 mM for AC2. A Lineweaver-Burk plot analysis demonstrated that the mode of inhibition of NKY80 was not competitive with respect to ATP or forskolin. These binding mechanisms are in agreement with that of P-site regulators.

Using the above established screening strategy, inventors re-screened a compound library and also newly synthesized compounds according to the pharmaco-design at the Millennium Pharmaceuticals, Inc. Among a few hundred candidates, inventors selected HI30435, N-7 linked adenine hydroxamate, for the detailed analysis. This compound significantly inhibited the catalytic activity of AC5 by 91% of control while to a lesser degree AC2 by 21% of control and AC3 by 28% of control (10 micro M). These data suggested that the class of compounds identified below act as selective inhibitors of AC5 in the striatum and thereby inhibited further cAMP signaling. This ratio of selectivity is much greater than NKY80, a previously identified AC5 inhibitor.

In one preferred embodiment the present invention relates to a compound of the formula (I): 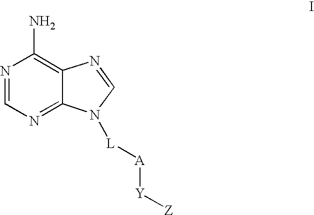
wherein:
A is a direct link or A is divalent member selected from the group consisting of:

phenyl, thienyl, furanyl, pyrrolyl, indolyl, 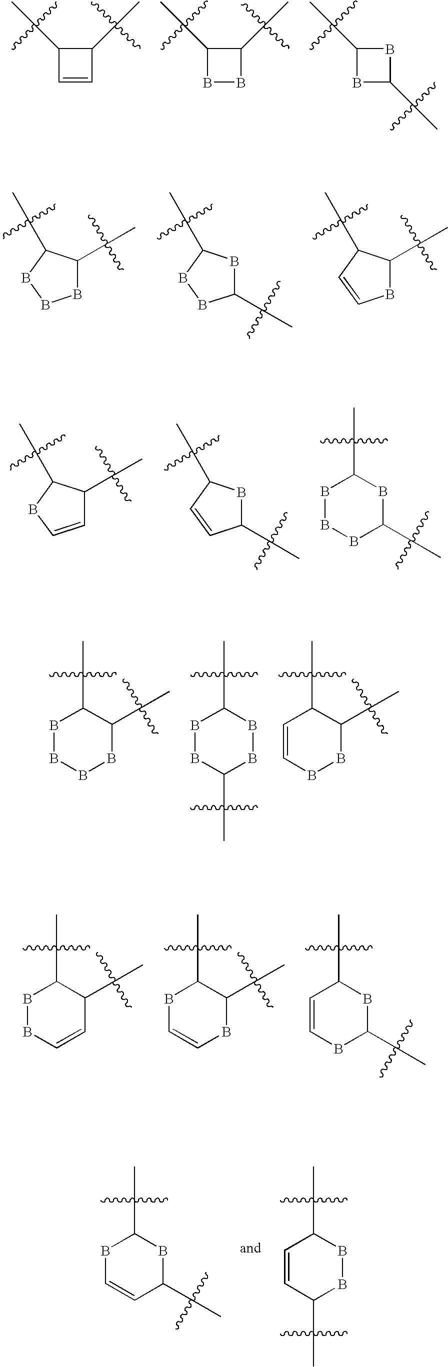
wherein
each B is independently —C(—R¹)(—R²)—, —O— or —N(-J-R³)—, and wherein only one ring B is either O or —N(-J-R³)—;
m and n are each independently an integer from 0-4;
q is an integer from 0 to 8;
Y is —(CH₂)_{q}—, —(CH₂)ₘO—, —(CH₂)ₘ—N(-J¹-)-R⁴;
Z is —(CH₂)ₙ—C(═O)—NHOH and —(CH₂)ₙCCOOH;
L is —(CH₂)_{q}—, —(CH₂)ₘO—, —(CH₂)ₘ—N-(-J²-)-R⁵;
J, J¹ and J² are each independently —C(═O)— or a direct link;
R¹ is H, —N(-J³-R⁶)(-J⁴-R⁷) or —O-J⁵-R⁸, wherein J³, J⁴ and J⁵ are each independently —C(═O)— or a direct link, and at least one of J³ and J⁴ is a direct link;
R² is H, —N(J⁶-R⁹)(-J⁷-R¹⁰) or —O-J⁸-R¹¹, wherein J⁶, J⁷ and J⁸ are each independently a —C(═O)— or a direct link, and at least one of J⁶ and J⁷ is a direct link;
R³ is H, C₁-C₈ alkyl, CF₃, or —O—R¹²;
R⁴ is H, C₁-C₈ alkyl, CF₃, or —O—R¹³;
R⁵ is H, C₁-C₈ alkyl, CF₃, or —O—R¹⁴;
R⁶ is H, C₁-C₈ alkyl, CF₃, or —O—R¹⁵;
R⁷ is H, C₁-C₈ alkyl, CF₃, or —O—R¹⁶;
R⁸ is H, C₁-C₈ alkyl, CF₃, or —O—R¹⁷;
R⁹ is H, C₁-C₈ alkyl, CF₃, or —O—R¹⁸;
R₁₀ is H, C₁-C₈ alkyl, CF₃, or —O—R¹⁹;
R₁₁ is H, C₁-C₈ alkyl, CF₃, or —O—R²⁰;
R¹², R¹³, R¹⁴, R¹⁵, R¹⁶, R¹⁷, R¹⁸, R¹⁹ and R²⁰ are each independently a C₁-C₄ alkyl, cycloalkyl or benzyl;
   and all pharmaceutically acceptable isomers, salts, hydrates, solvates and prodrug derivatives thereof.

The pharmaceutically acceptable salts of the compounds according to formula (I) include pharmaceutically acceptable acid addition salts, metal salts, ammonium salts, organic amine addition salts, amino acid addition salts, etc. Examples of the pharmaceutically acceptable acid addition salts of the compounds of formula (I) are inorganic acid addition salts such as hydrochloride, sulfate and phosphate, and organic acid addition salts such as acetate, maleate, fumarate, tartrate, citrate and methanesulfonate. Examples of the pharmaceutically acceptable metal salts are alkali metal salts such as sodium salt and potassium salt, alkaline earth metal salts such as magnesium salt and calcium salt, aluminum salt and zinc salt. Examples of the pharmaceutically acceptable ammonium salts are ammonium salt and tetramethyl ammonium salt. Examples of the pharmaceutically acceptable organic amine addition salts include heterocyclic amine salts such as morpholine and piperidine salts. Examples of the pharmaceutically acceptable amino acid addition salts are salts with lysine, glycine and phenylalanine.

This invention also encompasses prodrug derivatives of the compounds contained herein. Prodrug compounds of this invention may be called single, double, triple etc., depending on the number of biotransformation steps required to release the active drug within the organism, and indicating the number of functionalities present in a precursor-type form. Prodrug forms often offer advantages of solubility, tissue compatibility, or delayed release in the mammalian organism (Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, Calif., 1992). Prodrugs commonly known in the art include acid derivatives well known to practitioners of the art, such as, for example, esters prepared by reaction of the parent acids with a suitable alcohol, or amides prepared by reaction of the parent acid compound with an amine, or basic groups reacted to form an acylated base derivative. Moreover, the prodrug derivatives of this invention may be combined with other features herein taught to enhance bioavailability.

In the compounds of this invention, carbon atoms bonded to four non-identical substituents are asymmetric. Accordingly, the compounds may exist as diastereoisomers, enantiomers or mixtures thereof. The syntheses described herein may employ racemates, enantiomers or diastereomers as starting materials or intermediates. Diastereomeric products resulting from such syntheses may be separated by chromatographic or crystallization methods, or by other methods known in the art. Likewise, enantiomeric product mixtures may be separated using the same techniques or by other methods known in the art. Each of the asymmetric carbon atoms, when present in the compounds of this invention, may be in one of two configurations (R or S) and both are within the scope of the present invention.

In a second preferred embodiment, the invention provides compounds according to formula (I): 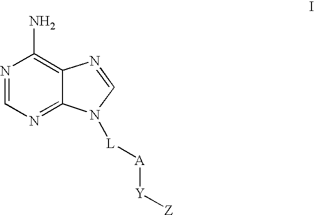
wherein:
A is a direct link, or
A is divalent member selected from the group consisting of: 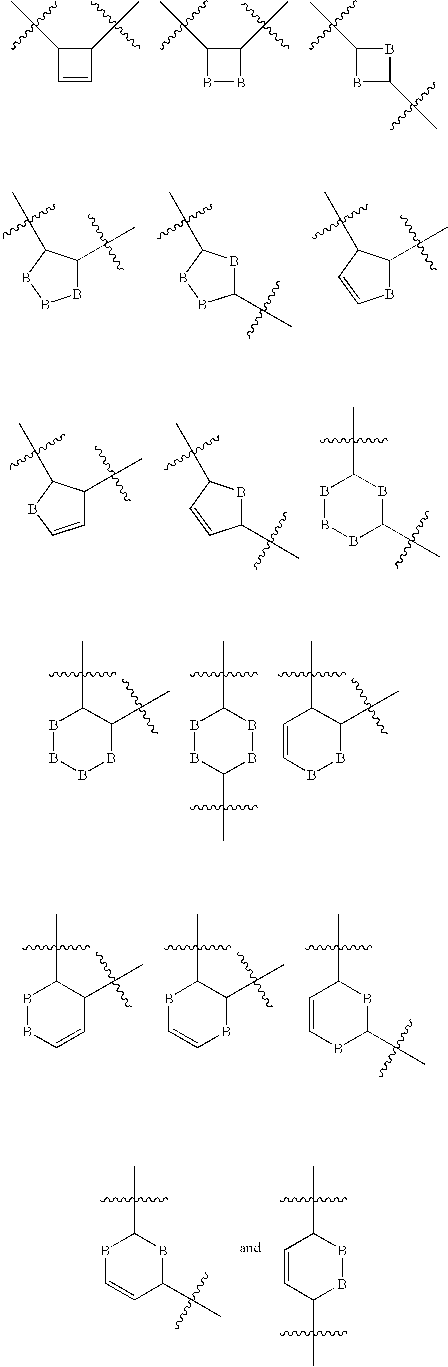
   wherein
each B is independently —C(—R¹)(—R²)—, —O— or —N(-J-R³)—, and wherein only one ring B is either O or —N(-J-R³)—;
m and n are each independently an integer from 0-4;
q is an integer from 0 to 8;
Y is a —(CH₂)_{q}— and —(CH₂)ₘO—;
Z is —(CH₂)ₙ—C(═O)—NHOH and —(CH₂)ₙCOOH;
L is —(CH₂)_{q}— and —(CH₂)ₘO—;
J is —C(═O)— or a direct link;
R¹ is H or —O-J⁵-R⁸, wherein J⁵ is a —C(═O)— or a direct link;
R² is a H or —O-J⁸-R¹, wherein J⁸ is —C(═O)— or a direct link;
R⁸ is H, C₁-C₈ alkyl, CF₃, or —O—R¹⁷;
R¹¹ is H, C₁-C₈ alkyl, CF₃, or —O—R²⁰;
R¹⁷ and R²⁰ are each independently a C₁-C₄ alkyl, cycloalkyl or benzyl;
   and all pharmaceutically acceptable isomers, salts, hydrates, solvates and prodrug derivatives thereof.

A third preferred embodiment is a compound of the formula (I): 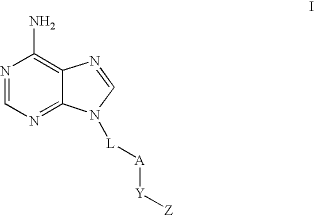
wherein:
A is divalent member selected from the group consisting of: 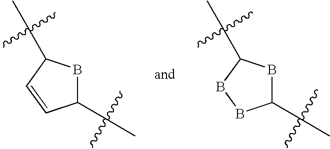
   wherein
each B is independently the substituted group —C(—R¹)(—R²)—;
Y is —(CH₂)_{q}— and —(CH₂)ₘO—;
Z is —(CH₂)ₙ—C(═O)—NHOH;
L is a —(CH₂)_{q}—;
m and n are each independently an integer from 0-4;
q is an integer from 0 to 8; and
R¹ and R² are each H;
   and all pharmaceutically acceptable isomers, salts, hydrates, solvates and prodrug derivatives thereof.

The compounds may be prepared using methods and procedures in the Examples presented herein. Starting materials may be made or obtained as described therein as well. Leaving groups such as halogen, lower alkoxy, lower alkylthio, lower alkylsulfonyloxy, arylsulfonyloxy, etc, may be utilized when necessary except for the reaction point, followed by deprotection. Suitable amino protective groups are those commonly known in the art such as ethoxycarbonyl, t-butoxycarbonyl, acetyl and benzyl. The protective groups can be introduced and eliminated according to conventional methods used in organic synthetic chemistry (T. W. Greene, Protective Groups in Organic Synthesis, John Wiley & Sons Inc. (1981)).

In such processes, if the defined groups change under the conditions of the working method or are not appropriate for carrying out the method, the desired compound can be obtained by using conventional organic synthetic methods for introducing and eliminating protective groups. Conversion of functional groups contained in the substituents can be carried out by known methods. (e.g., R. C. Larock, Comprehensive Organic Transformations (1989)), in addition to the above-described processes, and some of the active compounds of formula I may be utilized as intermediates for further synthesizing novel derivatives according to formula I.

The intermediates and the desired compounds in the processes described above can be isolated and purified by purification methods conventionally used in organic synthetic chemistry, for example, neutralization, filtration, extraction, washing, drying, concentration, recrystallization, and various kinds of chromatography. The intermediates may be subjected to the subsequent reaction without purification.

There may be tautomers for some compounds of formula I, and the present invention covers all possible isomers including tautomers and mixtures thereof. Where chiral carbons lend themselves to two different enantiomers, both enantiomers are contemplated as well as procedures for separating the two enantiomers. In the compounds of this invention, carbon atoms bonded to four non-identical substituents are asymmetric. Accordingly, the compounds may also exist as diastereoisomers, enantiomers or mixtures thereof. The syntheses described herein may employ racemates, enantiomers or diastereomers as starting materials or intermediates. Diastereomeric products resulting from such syntheses may be separated by chromatographic or crystallization methods, or by other methods known in the art. Likewise, enantiomeric product mixtures may be separated using the same techniques or by other methods known in the art. Each of the asymmetric carbon atoms, when present in the compounds of this invention, may be in one of two configurations (R or S) and both are within the scope of the present invention. In the processes described herein, the final products may, in some cases, contain a small amount of diastereomeric or enantiomeric products, however these products do not affect their therapeutic or diagnostic application.

In the case where a salt of a compound of formula I is desired and the compound is produced in the form of the desired salt, it can be subjected to purification as such. In the case where a compound of formula I is produced in the free state and its salt is desired, the compound of formula I is dissolved or suspended in a suitable organic solvent, followed by addition of an acid or a base to form a salt.

Formulations and Methods of Administration

A pharmaceutical composition useful in the present invention comprises an AC5 inhibitor and a pharmaceutically acceptable carrier, excipient, diluent and/or salt. Pharmaceutically acceptable carrier, diluent, excipient and/or salt means that the carrier, diluent, excipient and/or salt must be compatible with the other ingredients of the formulation, does not adversely affect the therapeutic benefit of the AC5 inhibitor, and is not deleterious to the recipient thereof.

Administration of the compounds or pharmaceutical compositions thereof for practicing the present invention can be by any method that delivers the compounds systemically. These methods include oral routes, parenteral routes, intraduodenal routes, etc.

For topical applications, the compound or pharmaceutical composition thereof can be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax, sugars such as lactose and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Depending on the particular condition, disorder or disease to be treated, additional therapeutic agents can be administered together with the AC5 inhibitor. Those additional agents can be administered sequentially in any order, as part of a multiple dosage regimen, from the AC5 inhibitor-containing composition (consecutive or intermittent administration). Alternatively, those agents can be part of a single dosage form, mixed together with the AC5 inhibitor in a single composition (simultaneous or concurrent administration).

For oral administration, a pharmaceutical composition useful in the invention can take the form of solutions, suspensions, tablets, pills, capsules, powders, granules, semisolids, sustained release formulations, elixirs, aerosols, and the like. Tablets containing various excipients such as sodium citrate, calcium carbonate and calcium phosphate are employed along with various disintegrants such as starch, preferably potato or tapioca starch, and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof. The choice of formulation depends on various factors such as the mode of drug administration (e.g., for oral administration, formulations in the form of tablets, pills or capsules are preferred) and the bioavailability of the drug substance.

A suitable pharmaceutical composition for parenteral injection can comprise pharmaceutically acceptable sterile aqueous or nonaqueous solutions, dispersions, suspensions or emulsions as well as sterile powders for reconstitution into sterile injectable solutions or dispersions just prior to use. Aqueous solutions are especially suitable for intravenous, intramuscular, subcutaneous and intraperitoneal injection purposes. In this connection, the sterile aqueous media employed are all readily obtainable by standard techniques well-known to those skilled in the art. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The pharmaceutical compositions useful in the present invention can also contain adjuvants such as, but not limited to, preservatives, wetting agents, emulsifying agents, and dispersing agents. Prevention of the action of microorganisms can be ensured by the inclusion of various antibacterial and antifungal agents, such as for example, paraben, chlorobutanol, phenol sorbic acid, and the like. It can also be desirable to include isotonic agents such as sugars, sodium chloride, and the like. Prolonged absorption of the injectable pharmaceutical form can be brought about by the inclusion of agents that delay absorption such as aluminum monostearate and gelatin.

Injectable depot forms are made by forming microencapsule matrices of the drug in biodegradable polymers such as polylactide, polyglycolide, and polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

For purposes of transdermal (e.g., topical) administration, dilute sterile, aqueous or partially aqueous solutions (usually in about 0.1% to 5% concentration), otherwise similar to the above parenteral solutions, are prepared.

The pharmaceutical compositions useful in the invention can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents.

In nonpressurized powder compositions, the active ingredients in finely divided form can be used in admixture with a larger-sized pharmaceutically acceptable inert carrier comprising particles having a size, for example, of up to 100 μm in diameter. Suitable inert carriers include sugars such as lactose. Desirably, at least 95% by weight of the particles of the active ingredient have an effective particle size in the range of 0.01 to 10 μm.

Alternatively, the composition can be pressurized and contain a compressed gas, such as, e.g., nitrogen, carbon dioxide or a liquefied gas propellant. The liquefied propellant medium and indeed the total composition are preferably such that the active ingredients do not dissolve therein to any substantial extent. The pressurized composition can also contain a surface active agent. The surface active agent can be a liquid or solid non-ionic surface active agent or can be a solid anionic surface active agent. It is preferred to use the solid anionic surface active agent in the form of a sodium salt.

Compositions for rectal or vaginal administration are preferably suppositories which can be prepared by mixing the compounds of the invention with suitable non-irritating excipients or carriers such as cocoa butter, polyethylene glycol or a suppository wax which are solid at room temperature but liquid at body temperature and therefore melt in the rectum or vaginal cavity and release the drugs.

The compositions useful in the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to the compounds of the invention, stabilizers, preservatives, excipients, and the like. The preferred lipids are the phospholipids and the phosphatidyl cholines (lecithins), both natural and synthetic. Methods to form liposomes are known in the art (e.g., Prescott, E., Meth. Cell Biol. 14:33 (1976)).

Other pharmaceutically acceptable carrier includes, but is not limited to, a non-toxic solid, semisolid or liquid filler, diluent, encapsulating material or formulation auxiliary of any type, including but not limited to ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Solid pharmaceutical excipients include, but are not limited to, starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients can be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Methods of preparing various pharmaceutical compositions with a certain amount of active ingredient are known, or will be apparent in light of this disclosure, to those skilled in this art. Other suitable pharmaceutical excipients and their formulations are described in Remington's Pharmaceutical Sciences, edited by E. W. Martin, Mack Publishing Company, 19th ed. (1995).

Pharmaceutical compositions useful in the present invention can contain 0.1%-95% of the compound(s) of this invention, preferably 1%-70%. In any event, the composition or formulation to be administered will contain a quantity of a compound(s) according to this invention in an amount effective to treat the condition, disorder or disease of the subject being treated.

One of ordinary skill in the art will appreciate that pharmaceutically effective amounts of the AC5 inhibitor can be determined empirically and can be employed in pure form or, where such forms exist, in pharmaceutically acceptable salt, ester or prodrug form. The agents can be administered to a patient as pharmaceutical compositions in combination with one or more pharmaceutically acceptable excipients. It will be understood that, when administered to, for example, a human patient, the total daily usage of the agents or composition of the present invention will be decided within the scope of sound medical judgement by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, sex and diet of the patient; the time of administration, route of administration, and rate of excretion of the agent; the duration of the treatment; drugs used in combination or coincidental with the specific agent; and like factors well known in the medical arts. For example, it is well within the skill of the art to start doses of the agents at levels lower than those required to achieve the desired therapeutic effect and to gradually increase the dosages until the desired effect is achieved.

For example, satisfactory results are obtained by oral administration of the compounds at dosages on the order of from 0.05 to 500 mg/kg/day, preferably 0.1 to 100 mg/kg/day, more preferably 1 to 50 mg/kg/day, administered once or, in divided doses, 2 to 4 times per day. On administration parenterally, for example, by i.v. bolus, drip or infusion, dosages on the order of from 0.01 to 1000 mg/kg/day, preferably 0.05 to 500 mg/kg/day, and more preferably 0.1 to 100 mg/kg/day, can be used. Suitable daily dosages for patients are thus on the order of from 2.5 to 500 mg p.o., preferably 5 to 250 mg p.o., more preferably 5 to 100 mg p.o., or on the order of from 0.5 to 250 mg i.v., preferably 2.5 to 125 mg i.v. and more preferably 2.5 to 50 mg i.v.

Dosaging can also be arranged in a patient specific manner to provide a predetermined concentration of the agents in the blood, as determined by techniques accepted and routine in the art (HPLC is preferred). Thus patient dosaging can be adjusted to achieve regular on-going blood levels, as measured by HPLC, on the order of from 50 to 5000 ng/ml, preferably 100 to 2500 ng/ml.

The following non-limiting reaction schemes demonstrate how compounds according to the Invention may be made.

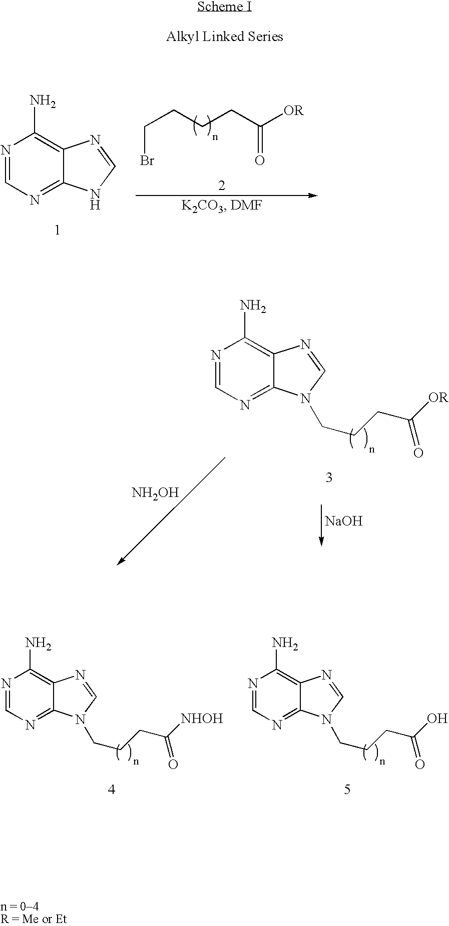

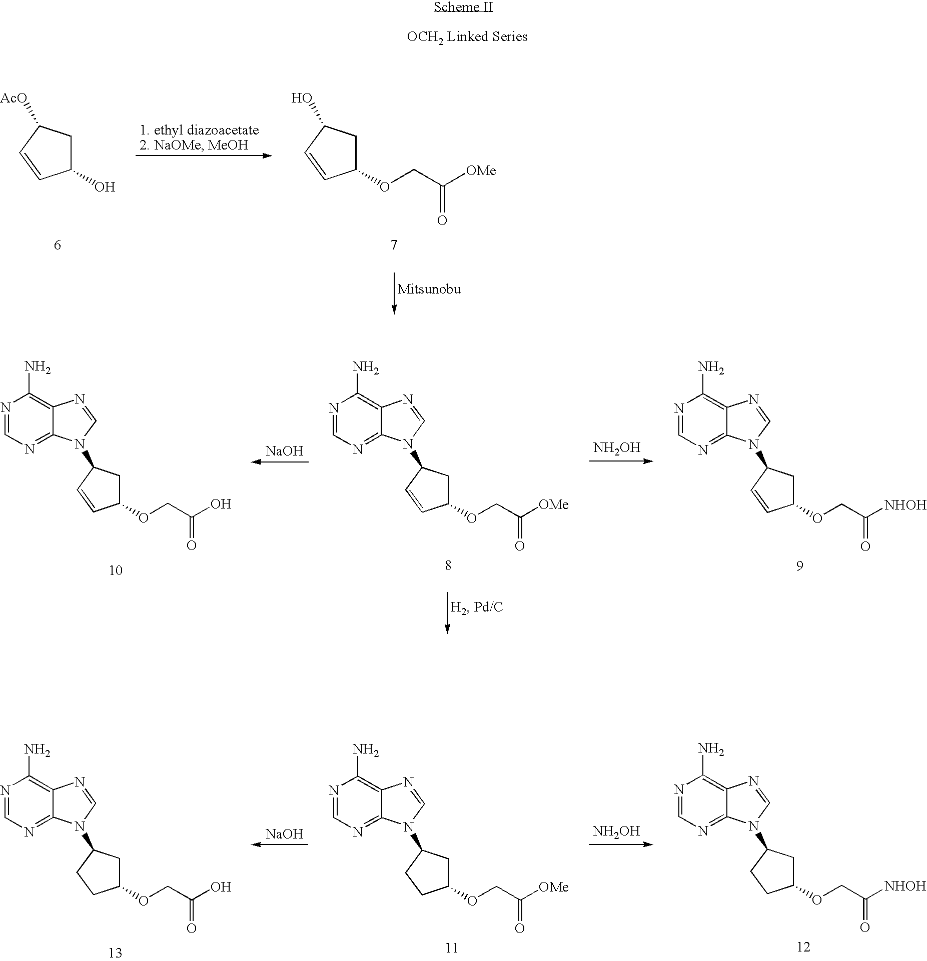

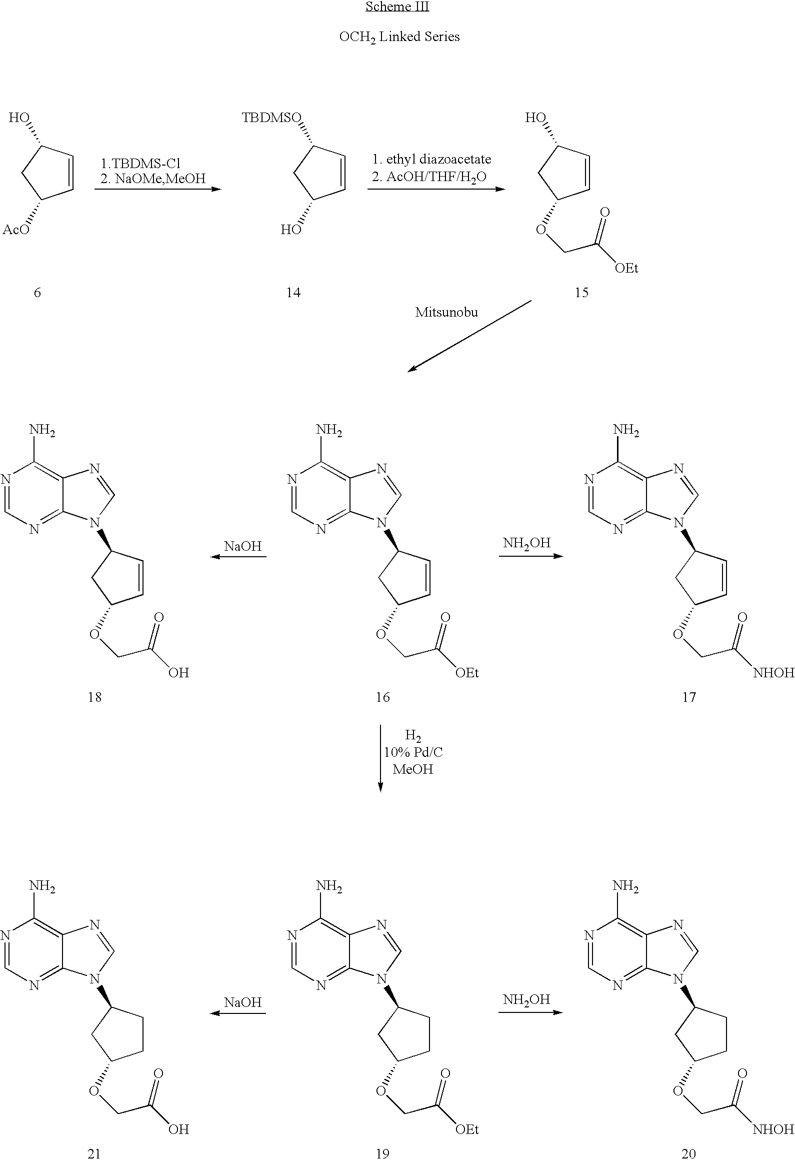

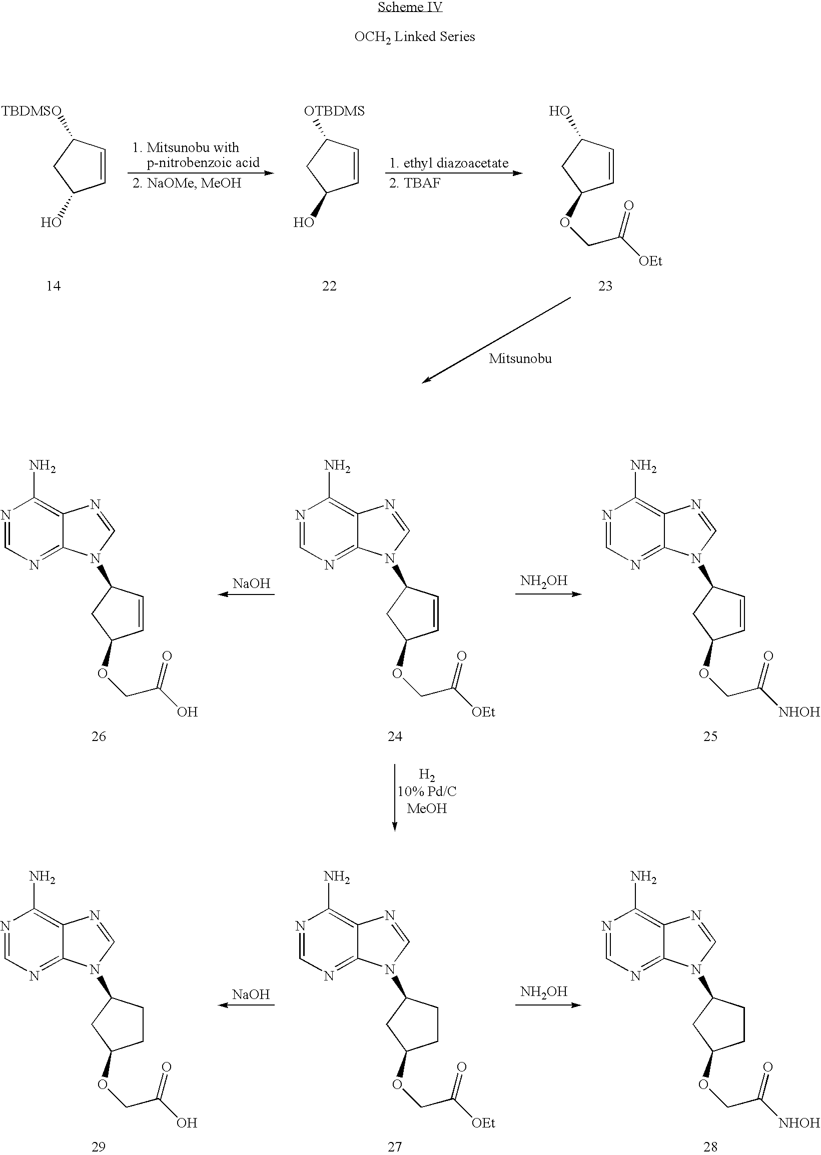

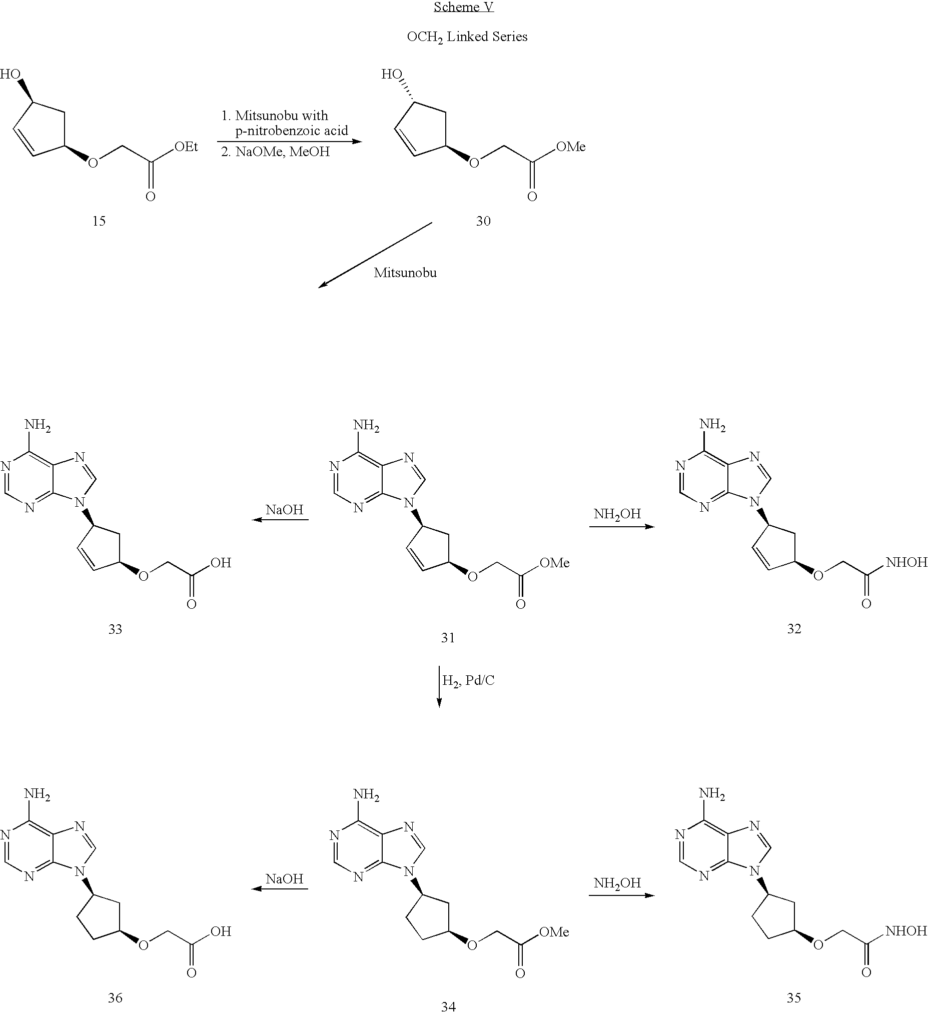

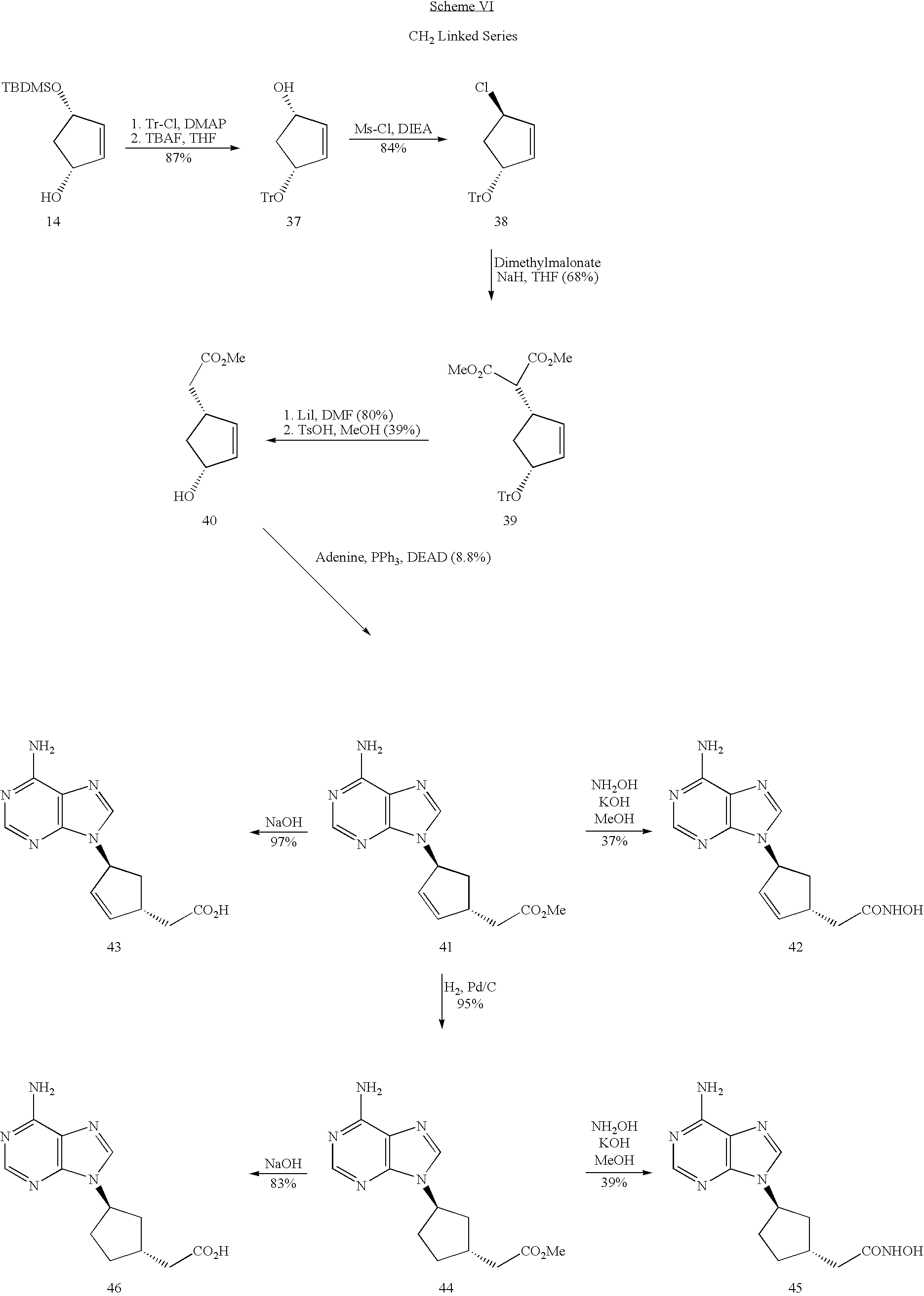

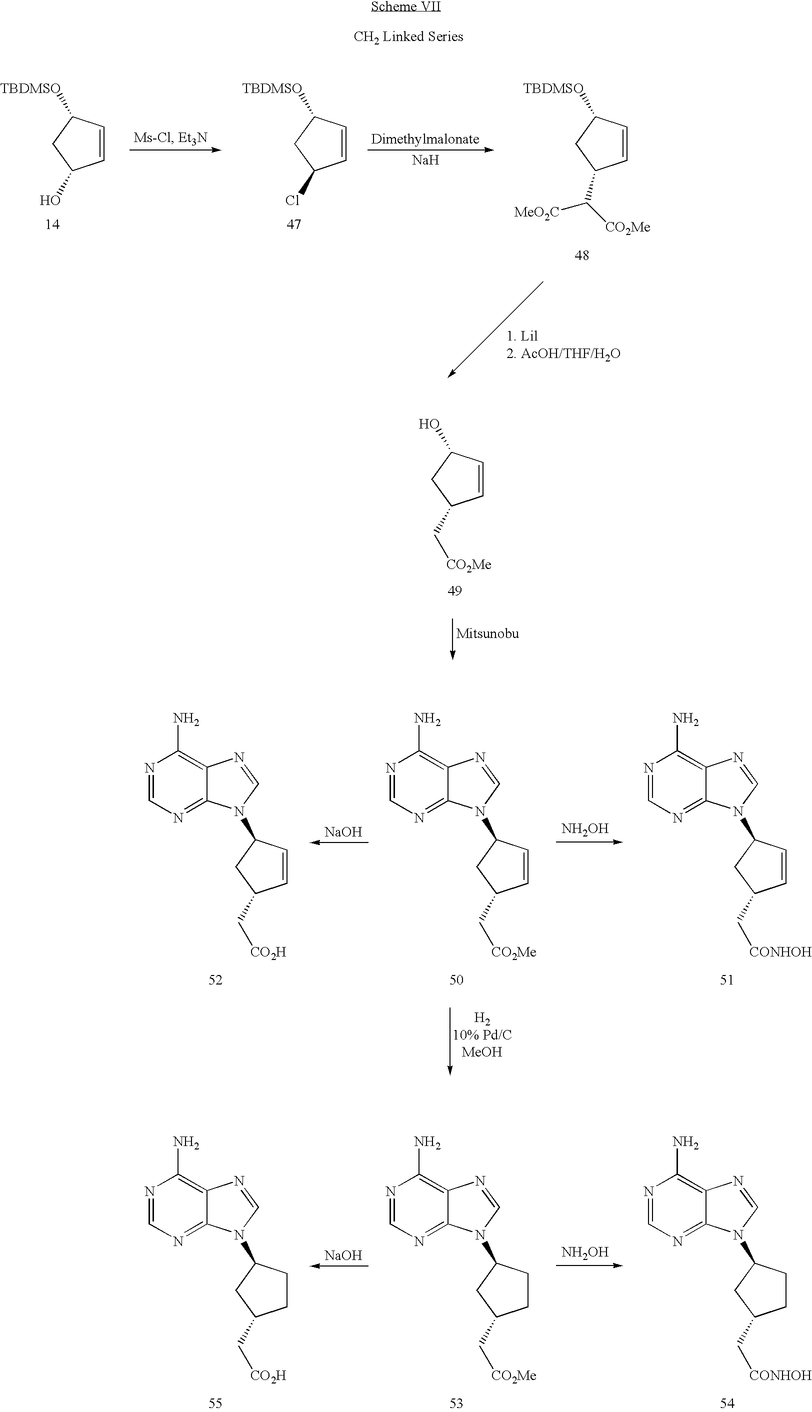

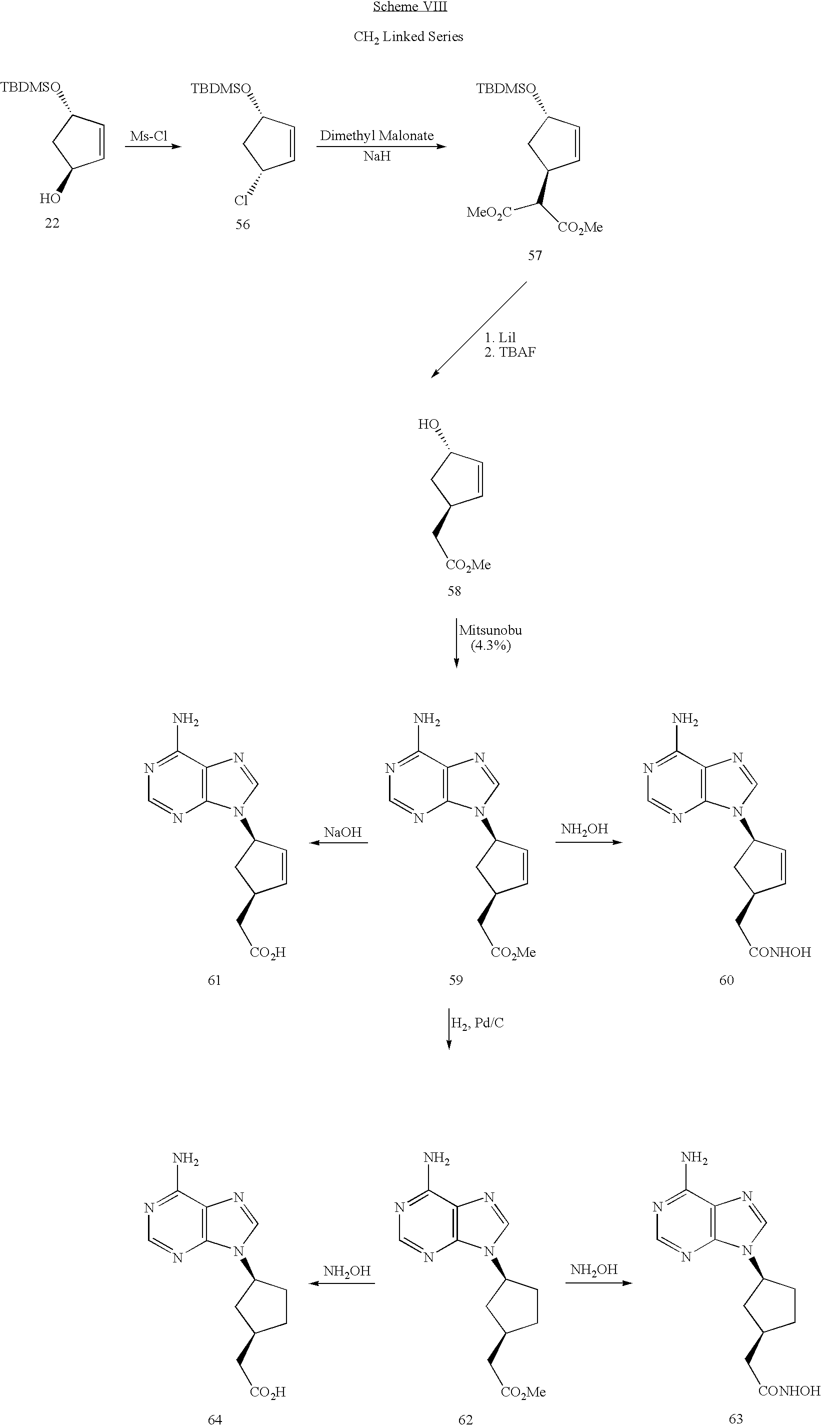

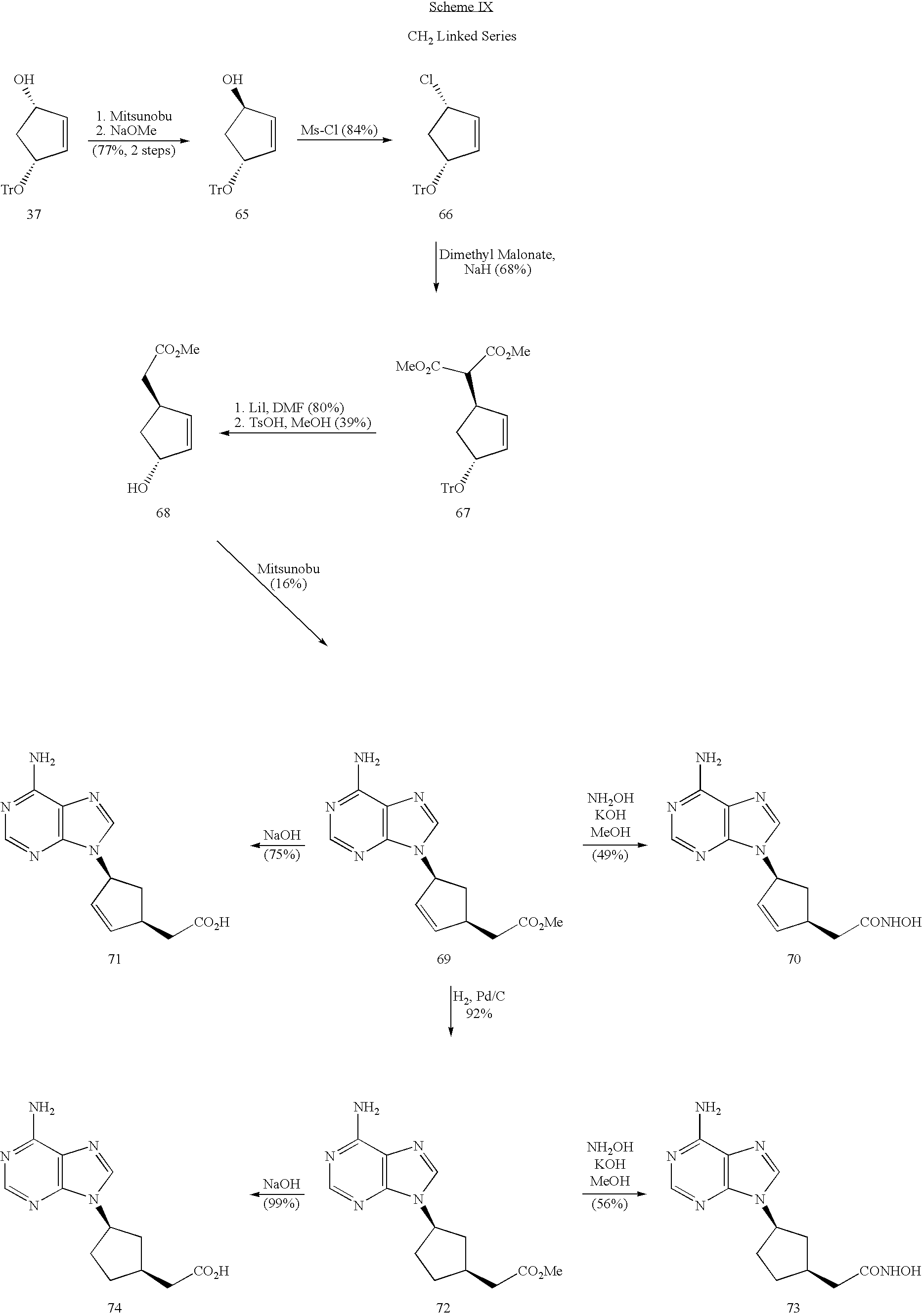

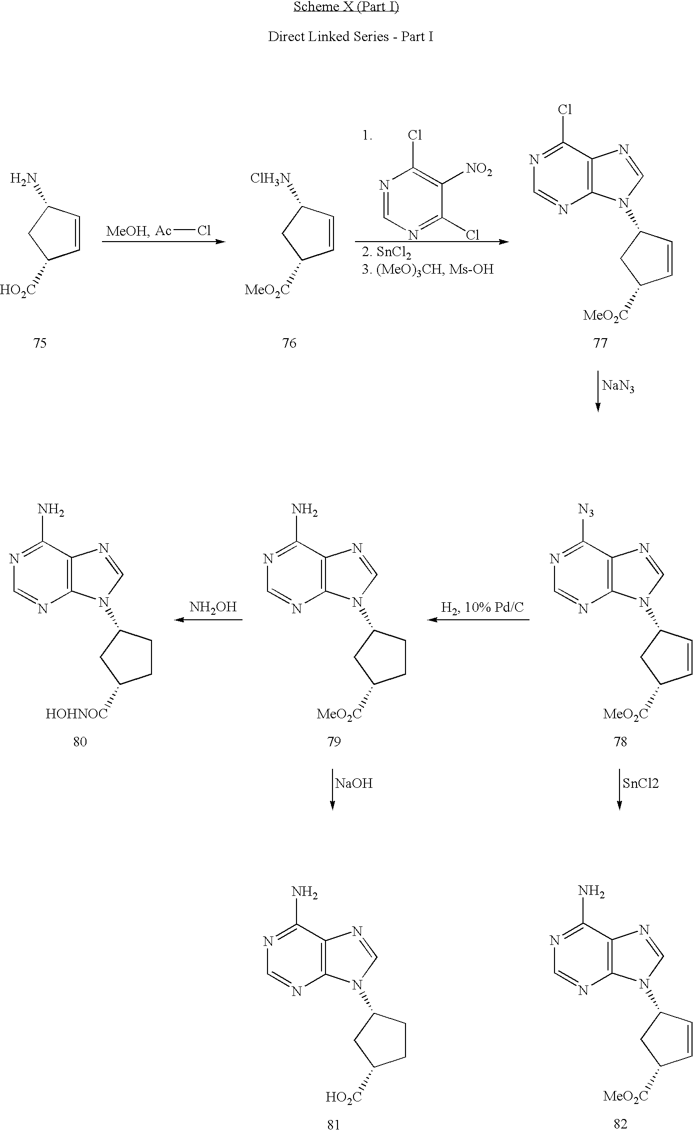

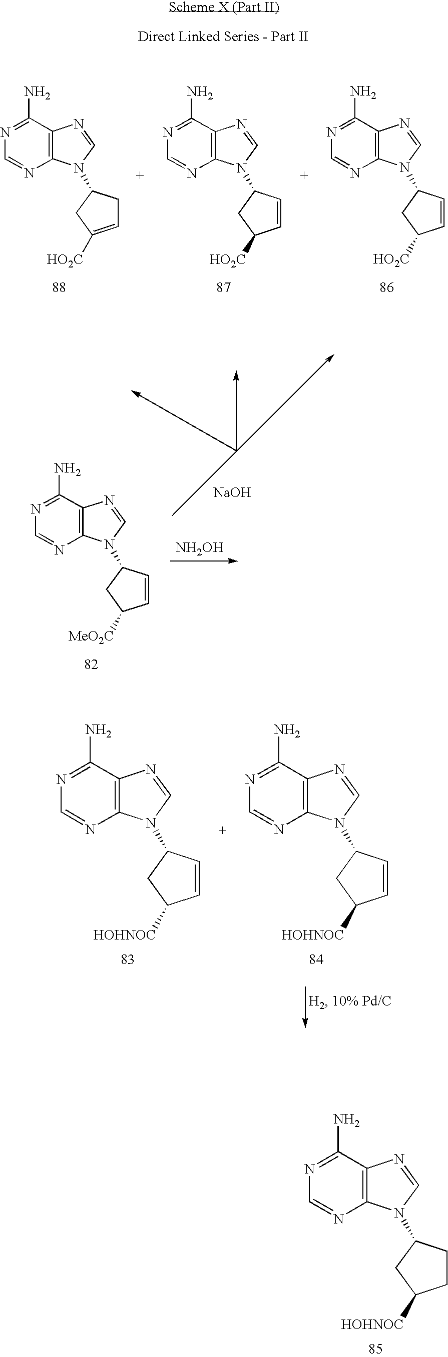

The following non-limiting Examples provide general chemical procedures for the synthesis of compounds according to the invention. While in no way intending to be bound by theory or limited, the following procedures refer to preparation of compounds according to Schemes I-X in order to more fully describe the invention.

### EXAMPLE 1

General Procedure A—Adenine Alkylations

Adenine (1.18 mmole) was combined with an alkyl bromide (3.54 mmoles), K₂CO₃ (5.91 mmoles) and DMF (5.00 mL). The mixture was heated to 60° C. for 20 hours. After cooling to room temperature, the reaction was diluted with brine (50 mL) and washed with EtOAc (3×20 mL). The combined organic washes were dried over anhydrous MgSO₄, filtered and concentrated to dryness. The product was purified on silica gel (5% MeOH/CHCl₃).

General Procedure B—Hydroxamic Acids

KOH (3.8 M in MeOH, 0.45 mL) was added to HONH₂.HCl (1.6 M in MeOH, 0.67 mL) and cooled to 0° C. for 2 hours. A methyl or ethyl ester (0.15 mmoles) was dissolved in MeOH (0.31 mL) and the HONH₂ solution was added by filtration. After stirring for 45 minutes at room temperature, the reaction was concentrated to dryness and the residue was purified by reverse phase preparative HPLC (0-10% CH₃CN/30 minutes). The isolated product was desalted with MP-carbonate resin (Argonaut) in MeOH, filtered and concentrated to dryness giving the desired hydroxamic acid.

General Procedure C—Carboxylic Acids

A methyl or ethyl ester (0.53 mmoles) was dissolved in MeOH (2.40 mL) and NaOH (2.00 M in H₂O, 1.60 mmoles) was added. The reaction was stirred at room temperature for 2.5 hours after which, it was acidified to pH=2 with DOWEX acid resin (50WX₂-100, MeOH washed). The reaction was filtered and concentrated to dryness giving the desired carboxylic acid.

General Procedure D—Rhodium Acetate

An alcohol (6.08 mmoles) was dissolved in CH₂Cl₂ (65 mL) and [Rh(OAc)2]2 (0.15 mmole) was added. Ethyl diazoacetate (13.30 mmoles) was added dropwise and the reaction was stirred at room temperature for 24 hours. After concentrating to dryness, the product was purified on silica gel (20% EtOAc/hexane).

General Procedure E—Acetate/p-nitrobenzoate Cleavage (NaOMe)

An acetate or p-nitrobenzoate (5.18 mmoles) was dissolved in anhydrous MeOH (15 mL) and catalytic NaOMe (solution in MeOH) was added. The reaction was stirred at room temperature for 24 hours after which, it was quenched with H2O (1.0 mL) and concentrated to dryness. The product was purified on silica gel (50% EtOAc/Hexane).

General Procedure F—Adenine Mitsunobu

An allylic alcohol (10.93 mmoles), triphenylphosphine (10.93 mmoles) and adenine (10.93 mmoles) were dissolved in THF (40 mL) and cooled to 0° C. Diethyl azodicarboxylate (10.93 mmoles) was added dropwise and the reaction was stirred at room temperature for 18 hours. After heating the reaction to 40° C. for an additional 4 hours, the mixture was cooled to room temperature and the solids were removed by filtration. The filtrate was concentrated to dryness and the residue was purified on silica gel (EtOAc then 5% MeOH/CHCl₃).

General Procedure G—Olefin Hydrogenation (Also Azide Reduction)

An olefin (100 mg) and 10% Pd/C (25 mg) were placed under Argon and MeOH (10 mL) was added. The mixture was degassed under vacuum and stirred under H₂ (1 atm) for 20 hours. The reaction was filtered and concentrated giving the desired product.

General Procedure H—TBDMS Protection

An alcohol (72.24 mmoles) was dissolved in THF (200 mL) and imidazole (108.36 mmoles) was added followed by TBDMS-Cl (90.30 mmoles). The reaction was stirred at room temperature for 24 hours after which, the solids were removed by filtration and the filtrate was concentrated to dryness. The residue was dissolved in EtOAc (300 mL) and washed with HCl (1 N, 3×50 mL), saturated NaHCO₃ (3×50 mL) and brine (50 mL). The organic phase was dried over anhydrous MgSO₄, filtered and concentrated and the residue was used with no further purification.

General Procedure I—TBDMS Cleavage (AcOH/THF/Water)

A TBDMS ether (4.52 mmoles) was combined with THF (1 mL), H₂O (1 mL) and acetic acid (3 mL). The reaction was stirred at room temperature for 6 hours after which, it was azeotroped with benzene (3×15 mL). The residue was dried under vacuum and purified on silica gel (25% EtOAc/Hexane).

General Procedure J—p-nitrobenzoic Acid Mitsunobu

An allylic alcohol (60.70 mmoles), p-nitrobenzoic acid (242.81 mmoles) and triphenylphospine (242.81 mmoles) were combined with THF (200 mL) and cooled to 0° C. under argon. Diethylazodicarboxylate (242.81 mmoles) was added dropwise and the reaction was stirred at room temperature for 15 hours and 40° C. for an additional 3 hours. After cooling to room temperature, the reaction was concentrated to dryness and the residue was diluted with EtOAc (200 mL). The resulting solution was washed with HCl (1 N, 3×50 mL), brine (50 mL), saturated NaHOC₃ (3×50 mL) and brine (50 mL). After drying over anhydrous MgSO₄, the organics were filtered, concentrated and stirred with Et₂O (150 mL) for 18 hours. The resulting solids were removed by filtration and the filtrate was concentrated to dryness. The isolated residue was used without further purification.

General Procedure K—TBDMS Cleavage (TBAF)

A TBDMS ether (69.58 mmoles) was dissolved in THF (500 mL) and tetrabutylammonium fluoride (1 M in THF, 104 mL) was added. The reaction was stirred at room temperature for 2 hours and concentrated to dryness. The residue was filtered through silica gel (EtOAc) and again concentrated to dryness. Final purification was achieved on silica gel (10% then 25% then 50% EtOAc/Hexane).

General Procedure L—Allyl Chloride

An allylic alcohol (46.17 mmoles) was dissolved in CH₂Cl₂ and diisopropylethyl-amine (69.25 mmoles) was added. The resulting solution was cooled to 0° C. under argon and methanesulfonyl chloride (57.71 mmoles) was added. After stirring at 0° C. for 3 hours, the reaction was diluted with EtOAc (600 mL). The mixture was then washed with HCl (1 N, 3×50 mL), saturated NaHCO₃ (3×50 mL) and brine (50 mL). The organics were dried over anhydrous MgSO₄, filtered and concentrated and the residue was purified on silica gel (5% EtOAc/Hexane).

General Procedure M—Malonate Coupling

NaH (60%, 149.03 mmoles) was suspended in anhydrous THF (400 mL) and cooled to 0° C. under argon. Dimethylmalonate (149.03 mmoles) was added dropwise over 30 minutes and the reaction was allowed to warm to room temperature. An allyl chloride (29.81 mmoles) was dissolved in anhydrous THF (100 mL) and added to the malonate solution via cannula. After heating to 75° C. for 19 hours, the reaction was cooled, concentrated to a volume of 150 mL and diluted with 50% EtOAc/Hexane (300 mL). The resulting solution was washed with saturated NH₄Cl (3×50 mL) and brine (2×50 mL). Following concentration, the organics were partitioned between hexane (150 mL) and H₂O (150 mL). The hexane layer was further washed with H₂O (2×50 mL), dried over anhydrous MgSO₄, filtered and concentrated. The residue was purified on silica gel (5% EtOAc/Hexane).

General Procedure N—Decarboxylation (Lil)

A substituted malonate (31.93 mmoles) was combined with Lil (191.58 mmoles) and dissolved in DMF (260 mL). The mixture was degassed under vacuum, placed under argon and heated to 130° C. for 17 hours. After cooling to room temperature, the reaction was diluted with 25% EtOAc/Hexane (1500 mL) and washed with H₂O (3×300 mL) and brine (100 mL). The organic phase was dried over anhydrous MgSO₄, filtered and concentrated. The resulting residue was purified on silica gel (5% EtOAc/Hexane).

General Procedure O—Tritylation

An allylic alcohol (9.39 mmoles), trityl chloride (46.96 mmoles) and DMAP (56.36 mmoles) were combined and dissolved in DMF (30 mL). After heating to 100° C. for 20 hours, the reaction was cooled to room temperature and diluted with H2O (200 mL). The aqueous mixture was washed with 50% EtOAc/Hexane (200 mL) and the organics were sequentially washed with HCl (1 N, 3×25 mL), saturated NaHCO₃ (3×25 mL) and brine (25 mL). The organics were dried over MgSO₄, filtered, concentrated to dryness and used without further purification.

General Procedure P—Trityl Cleavacie (TsOH)

A trityl ether (15.75 mmoles) was dissolved in MeOH (100 mL) and p-toluene-sulfonic acid (0.79 mmoles) was added. After stirring at room temperature for 1.25 hours, the reaction was quenched with saturated NaHCO₃ (100 mL). The resulting mixture was washed with EtOAc (3×100 mL) and the combined organic extracts were washed with brine (50 mL). After drying over anhydrous MgSO₄, the product was purified on silica gel (25% then 50% EtOAc/Hexane).

General Procedure Q—Methyl Ester Formation (MeOH, Ac—Cl)

Acetyl chloride (9.00 mmoles) was slowly added to MeOH (35.00 mL) and cooled to 0° C. A carboxylic acid (7.87 mmoles) was added and the resulting mixture was stirred at room temperature for 4 hours. Concentration of the reaction mixture provided the desired product requiring no further purification.

General Procedure R—Coupling With Pyrimidine

An amine hydrochloride (7.97 mmoles) was combined with dichloronitro-pyrimidine (11.95 mmoles) and EtOH (80 mL). Triethylamine (23.90 mmoles) was added and the reaction was stirred at room temperature for 3.5 hours. Following dilution with EtOAc (320 mL), the mixture was sequentially washed with HCl (1 N, 3×50 mL), saturated NaHCO₃ (3×30 mL) and brine (30 mL). The organics were dried over anhydrous MgSO₄, filtered and concentrated. The isolated residue was used with no further purification.

General Procedure S—Nitro Group Reduction (SnCl₂)

A nitropyrimidine (9.36 mmoles) was dissolved in EtOH (75 mL) and SnCl₂ (28.09 mmoles) was added. The reaction was heated to reflux for 50 minutes and cooled to room temperature. Following quenching with saturated NaHCO₃ (300 mL), the reaction was washed with EtOAc (3×75 mL). The organic extracts were washed with brine (2×75 mL), dried over anhydrous MgSO₄, filtered and concentrated. No further purification was required.

General Procedure T—Purine Formation (orthoformate, Ms-OH)

A diaminopyrimidine (9.36 mmoles) was dissolved in trimethylorthoformate (25 mL) and methanesulfonic acid (0.22 mL) was added. The reaction was stirred at room temperature for 4.5 hours and diluted with EtOAc (150 mL). The resulting mixture was washed with saturated NaHCO₃ (3×25 mL) and brine (25 mL). The organic phase was dried over anhydrous MgSO₄, filtered and concentrated. The product was purified on silica gel (50% EtOAc/Hexane).

General Procedure U—Azidopurine

A chloropurine (3.02 mmoles), sodium azide (9.06 mmoles), EtOH (13 mL) and H₂O (6.5 mL) were combined and heated to 50° C. for 20 hours. After stirring for an additional 17 hours at room temperature, the reaction was concentrated to dryness. The residue was diluted with H₂O (20 mL) and the resulting solids were filtered, washed with H₂O and dried in a dessicator. No further purification was required.

General Procedure V—Azide Reduction (SnCl₂)

An azidopurine (0.42 mmoles) was dissolved in EtOH (3.25 mL) and SnCl₂ (1.27 mmoles) was added. The reaction was heated to reflux for 20 minutes and cooled to room temperature. Following quenching with saturated NaHCO₃ (15 mL), the reaction was washed with EtOAc (3×15 mL). The organic extracts were dried over anhydrous MgSO4, filtered and concentrated. No further purification was required.

HPLC Methods

A 10%-90% CH₃CN/10 minutes

B 0%-90% CH₃CN/10 minutes

C 5%-85% CH₃CN/9 minutes

### ethyl-3-(9-adenenyl)-propionoate (3a)

Compound 3a was prepared by coupling adenine with methylbromopropionate according to general procedure A. Yield=7%. TLC: R_{f}=0.17 (5% MeOH/CHCl₃). ¹H NMR (400 MHz, DMSO): δ 3.10 (t, 2H), 3.70 (s, 3H), 4.50 (t, 2H), 7.30 (s, 2H), 8.20 (s, 1H), 8.23 (s, 1H).

### Ethyl-4-(9-adenenyl)-butyrate (3b)

Compound 3b was prepared by coupling adenine with ethylbromobutyrate according to general procedure A. Yield=60%. TLC: R_{f}=0.15 (5% MeOH/CHCl₃). ¹H NMR (400 MHz, DMSO): δ 1.25 (t, 3H), 2.15 (m, 2H), 2.40 (t, 2H), 4.10 (q, 2H), 4.30 (t, 2H), 7.30 (s, 2H), 8.23 (s, 1H), 8.25 (s, 1H).

### Methyl-5-(9-adenenyl)-pentanoate (3c)

Compound 3c was prepared by coupling adenine with methylbromopentanoate according to general procedure A. Yield=40%. TLC: R_{f}=0.26 (5% MeOH/CHCl₃). Purity: >95% (HPLC method A). ¹H NMR (400 MHz, DMSO): δ 1.58 (m, 2H), 1.95 (m, 2H), 2.45 (t, 2H), 3.65 (s, 3H), 4.25 (t, 2H), 7.30 (s, 2H), 8.23 (s, 1H), 8.25 (s, 1H).

### Ethyl-6-(9-adenenyl)-hexanoate (3d)

Compound 3d was prepared by coupling adenine with ethylbromohexanoate according to general procedure A. Yield=55%. TLC: R_{f}=0.22 (5% MeOH/CHCl₃). Purity: >90% (HPLC method A). ¹H NMR (400 MHz, DMSO): δ 1.25 (t, 3H), 1.35 (m, 2H), 1.65 (m, 2H), 1.90 (m, 2H), 2.35 (t, 2H), 4.10 (q, 2H), 4.25 (t, 2H), 7.30 (s, 2H), 8.23 (s, 1H), 8.25 (s, 1H).

### Ethyl-7-(9-adenenyl)-heptanoate (3e)

Compound 3e was prepared by coupling adenine with ethylbromoheptanoate according to general procedure A. Yield=39%. TLC: R_{f}=0.25 (5% MeOH/CHCl₃). Purity: >95% (HPLC method A). ¹H NMR (400 MHz, DMSO): δ 1.25 (t, 3H), 1.35 (m, 4H), 1.60 (m, 2H), 1.90 (m, 2H), 2.35 (t, 2H), 4.15 (q, 2H), 4.25 (t, 2H), 7.30 (s, 2H), 8.24 (s, 1H), 8.25 (s, 1H).

### N-Hydroxy-3-(9-adenenyl)-propionamide (4a)

Compound 4a was prepared by subjecting compound 3a to general procedure B. Yield=60%. TLC: R_{f}=0.17 (CHCl₃/MeOH/H₂O 150/45/5). Purity: >95% (HPLC method C). ¹H NMR (400 MHz, DMSO): δ 2.70 (t, 2H), 4.45 (t, 2H), 7.30 (s, 2H), 8.10 (s, 1H), 8.25 (s, 1H), 8.90 (s, 1H), 10.60 (s, 1H).

### N-Hydroxy-4-(9-adenenyl)-butyramide (4b)

Compound 4b was prepared by subjecting compound 3b to general procedure B. Yield=68%. TLC: R_{f}=0.19 (CHCl₃/MeOH/H₂O 150/45/5). Purity: >99% (HPLC method B). ¹H NMR (400 MHz, DMSO): δ 2.05 (m, 2H), 2.15 (m, 2H), 4.25 (t, 2H), 7.30 (s, 2H), 8.25 (s, 2H), 8.85 (s, 1H), 10.50 (s, 1H).

### N-Hydroxy -5-(9-adenenyl)-pentanamide (4c)

Compound 4c was prepared by subjecting compound 3c to general procedure B. Yield=74%. TLC: R_{f}=0.24 (CHCl₃/MeOH/H₂O 150/45/5). Purity: >90% (HPLC method A). ¹H NMR (400 MHz, DMSO): δ 1.50 (m, 2H), 1.90 (m, 2H), 2.10 (t, 2H), 4.20 (t, 2H), 7.30 (s, 2H), 8.20 (s, 2H), 8.75 (s, 1H), 10.40 (s, 1H).

### N-Hydroxy -6-(9-adenenyl)-hexanamide (4d)

Compound 4d was prepared by subjecting compound 3d to general procedure B. Yield=80%. TLC: R_{f}=0.32 (CHCl₃/MeOH/H₂O 150/45/5). Purity: >90% (HPLC method A). ¹H NMR (400 MHz, DMSO): δ 1.30 (m, 2H), 1.60 (m, 2H), 1.90 (m, 2H), 2.00 (t, 2H), 4.20 (t, 2H), 7.30 (s, 2H), 8.20 (s, 2H), 8.75 (s, 1H), 10.40 (s, 1H).

### N-Hydroxy -7-(9-adenenyl)-heptanamide (4e)

Compound 4e was prepared by subjecting compound 3e to general procedure B. Yield=93%. TLC: R_{f}=0.42 (CDCl₃/MeOH/H₂O 150/45/5). Purity: >90% (HPLC method A). ¹H NMR (400 MHz, DMSO): δ 61.30 (m, 4H), 1.55 (m, 2H), 1.90 (m, 2H), 2.00 (t, 2H), 4.20 (t, 2H), 7.30 (s, 2H), 8.20 (s, 2H), 8.75 (s, 1H), 10.40 (s, 1H).

### 5-(9-Adenenyl)-pentanoic acid (5c)

Compound 5c was prepared by subjecting compound 3c to general procedure C. Yield=26%. Purity: >95% (HPLC method A). ¹H NMR (400 MHz, DMSO): δ 1.50 (m, 2H), 1.90 (m, 2H), 2.35 (t, 2H), 4.20 (t, 2H), 7.30 (s, 2H), 8.21 (s, 1H), 8.22 (s, 1H), 12.2 (bs, 1H).

### 6-(9-Adenenyl)-hexanoic acid (5d)

Compound 5d was prepared by subjecting compound 3d to general procedure C. Yield=31%. Purity: >95% (HPLC method A). ¹H NMR (400 MHz, DMSO): δ 1.35 (m, 2H), 1.60 (m, 2H), 1.90 (m, 2H), 2.30 (t, 2H), 4.20 (t, 2H), 7.30 (s, 2H), 8.21 (s, 1H), 8.22 (s, 1H), 12.2 (bs, 1H).

### 7-(9-Adenenyl)-heptanoic acid (5e)

Compound 5e was prepared by subjecting compound 3e to general procedure C. Yield=31%. Purity: >95% (HPLC method A). ¹H NMR (400 MHz, DMSO): δ 1.35 (m, 4H), 1.55 (m, 2H), 1.90 (m, 2H), 2.30 (t, 2H), 4.20 (t, 2H), 7.30 (s, 2H), 8.21 (s, 1H), 8.22 (s, 1H), 12.1 (s, 1H).

### (1R,3S)-1-Hydroxy-3-(methyl-carboxymethoxy)-4-cyclopentene (7)

(1R,3S)-1-Acetoxy-3-hydroxy-4-cyclopentene was subjected to general procedure D. Yield=85%. TLC: R_{f}=0.33 (25% EtOAc/Hexane). ¹H NMR (400 MHz, CDCl₃): δ 1.25 (t, 3H), 1.75 (m, 1H), 2.05 (s, 3H), 2.75 (m, 1H), 4.10 (s, 2H), 4.20 (q, 2H), 4.55 (m, 1H), 5.45 (m, 1H), 6.00 (d, 1H), 6.15 (d, 1H). Subsequent subjection of the product to general procedure E gave compound 7. Yield=84%. TLC: R_{f}=0.59 (EtOAc). ¹H NMR (400 MHz, CDCl₃): δ 1.70 (s, 2H), 2.65 (m, 1H), 3.75 (s, 3H), 4.10 (s, 2H), 4.45 (m, 1H), 4.65 (m, 1H), 6.05 (m, 2H).

### (1S,3S)-1-(9-Adenenyl)-3-(methyl-carboxymethoxy)-4-cyclopentene (8)

Compound 8 was prepared by subjecting compound 7 to general procedure F. Yield=17%. TLC: R_{f}=0.16 (5% MeOH/CHCl₃). Purity: >93% (HPLC method C). ¹H NMR (400 MHz, DMSO): δ 2.45 (m, 2H), 3.80 (s, 3H), 4.35 (s, 2H), 5.10 (m, 1H), 5.85 (m, 1H), 6.25 (m, 1H), 6.45 (m, 1H), 7.35 (s, 2H), 8.15 (s, 1H), 8.25 (s, 1H).

### (1S,3S)-1-(9-Adenenyl)-3-(N-hydroxycarbamoylmethoxy)-4-cyclopentene (9)

Compound 9 was prepared by subjecting compound 8 to general procedure B. Yield=95%. TLC: R_{f}=0.33 (CHCl₃/MeOH/H₂O 150/45/5). Purity: >91% (HPLC method C). ¹H NMR (400 MHz, DMSO): δ 2.45 (m, 1H), 2.55 (m, 1H), 4.05 (s, 2H), 5.05 (m, 1H), 5.90 (m, 1H), 6.30 (m, 1H), 6.50 (m, 1H), 7.95 (bs, 2H), 8.25 (s, 1H), 8.35 (s, 1H), 8.95 (s, 1H), 10.70 (s, 1H).

### (1S,3S)-1-(9-Adenenyl)-3-carboxymethoxy-4-cyclopentene (10)

Compound 10 was prepared by subjecting compound 8 to general procedure C. Yield=52%. TLC: R_{f}=0.07 (CHCl₃/MeOH/H₂O 150/45/5). Purity: >99% (HPLC method C). ¹H NMR (400 MHz, DMSO): δ 2.45 (m, 2H), 4.10 (s, 2H), 5.10 (m, 1H), 5.85 (m, 1H), 6.25 (m, 1H), 6.45 (m, 1H), 7.35 (s, 2H), 8.15 (s, 1H), 8.25 (s, 1H).

### (1R,3R)-1-(9-Adenenyl)-3-(methyl-carboxymethoxy)cyclopentane (11)

Compound 11 was prepared by subjecting compound 8 to general procedure G. Yield=85%. TLC: R_{f}=0.33 (CHCl₃/MeOH/H₂O 150/45/5). Purity: >95% (HPLC method C). ¹H NMR (400 MHz, DMSO): δ 1.90 (m, 1H), 2.10 (m, 1H), 2.35 (m, 4H), 3.80 (s, 3H), 4.25 (s, 2H), 4.35 (m, 1H), 5.10 (m, 1H), 7.30 (s, 2H), 8.20 (s, 1H), 8.35 (s, 1H).

### (1R,3R)-1-(9-Adenenyl)-3-(N-hydroxycarbamoylmethoxy)cyclopentane (12)

Compound 12 was prepared by subjecting compound 11 to general procedure B. Yield=99%. TLC: R_{f}=0.22 (5% MeOH/CHCl₃). Purity: >99% (HPLC method C). ¹H NMR (400 MHz, DMSO): δ 1.90 (m, 1H), 2.10 (m, 1H), 2.35 (m, 4H), 3.95 (s, 2H), 4.30 (m, 1H), 5.10 (m, 1H), 7.35 (s, 2H), 8.20 (s, 1H), 8.35 (s, 1H), 8.95 (s, 1H), 10.65 (s, 1H).

### (1R,3R)-1-(9-Adenenyl)-3-carboxymethoxycyclopentane (13)

Compound 13 was prepared by subjecting compound 11 to general procedure C. Yield=65%. TLC: R_{f}=0.07 (CHCl₃/MeOH/H₂O 150/45/5). Purity: >99% (HPLC method C). ¹H NMR (400 MHz, DMSO): δ 1.90 (m, 1H), 2.10 (m, 1H), 2.35 (m, 4H), 4.15 (s, 2H), 4.35 (m, 1H), 5.10 (m, 1H), 7.30 (s, 2H), 8.25 (s, 1H), 8.35 (s, 1H).

### (1R,3S)-1-Hydroxy-3-(tert-Butyl-dimethylsiloxy)-4-cyclopentene (14)

(1R,3S)-1-Acetoxy-3-hydroxy-4-cyclopentene to general procedure H. Yield=100%. TLC: R_{f}=0.48 (10% EtOAc/Hexane). ¹H NMR (400 MHz, CDCl₃): δ 0.05 (s, 6H), 0.90 (s, 9H), 1.55 (m, 1H), 2.00 (s, 3H), 2.80 (m, 1H), 4.70 (m, 1H), 5.45 (m, 1H), 5.85 (m, 1H), 5.95 (m, 1H). Subsequent subjection of the product to general procedure E gave compound 14. Yield=90%. TLC: R_{f}=0.43 (25% EtOAc/Hexane). ¹H NMR (400 MHz, CDCl₃): δ 0.05 (s, 6H), 0.90 (s, 9H), 1.50 (m, 1H), 2.65 (m, 1H), 4.55 (m, 1H), 4.65 (m, 1H), 5.85 (m, 1H), 5.95 (m, 1H).

### (1S,3R)-1-Hydroxy-3-(ethyl-carboxymethoxy)-4-cyclopentene (15)

Compound 14 was subjected to general procedure D. Yield=77%. TLC: R_{f}=0.29 (10% EtOAc/Hexane). ¹H NMR (400 MHz, CDCl₃): δ 0.05 (s, 6H), 0.90 (s, 9H), 1.25 (t, 3H), 1.60 (m, 1H), 2.65 (m, 1H), 4.10 (s, 2H), 4.20 (q, 2H), 4.55 (m, 1H), 4.65 (m, 1H), 5.95 (m, 2H). Subsequent subjection of the product to general procedure I gave compound 15. Yield=79%. TLC: R_{f}=0.65 (EtOAc). ¹H NMR (400 MHz, CDCl₃): δ 1.25 (t, 3H), 1.65 (m, 1H), 1.75 (s, 1H), 2.65 (m, 1H), 4.10 (s, 2H), 4.20 (q, 2H), 4.45 (m, 1H), 4.65 (m, 1H), 6.05 (m, 2H).

### (1R,3R)-1-(9-Adenenyl)-3-(ethyl-carboxymethoxy)-4-cyclopentene (16)

Compound 16 was prepared by subjecting compound 15 to general procedure F. Yield=21%. TLC: R_{f}=0.18 (5% MeOH/CHCl₃). Purity: >95% (HPLC method C). ¹H NMR (400 MHz, DMSO): δ 1.35 (t, 3H), 2.45 (m, 2H), 4.25 (q, 2H), 4.35 (s, 2H), 5.10 (m, 1H), 5.85 (m, 1H), 6.25 (m, 1H), 6.45 (m, 1H), 7.35 (s, 2H), 8.15 (s, 1H), 8.25 (s, 1H).

### (1R,3R)-1-(9-Adenenyl)-3-(N-hydroxycarbamoylmethoxy)-4-cyclopentene (17)

Compound 17 was prepared by subjecting compound 16 to general procedure B. Yield=88%. TLC: R_{f}=0.33 (CHCl₃/MeOH/H₂O 150/45/5). Purity: >95% (HPLC method C). ¹H NMR (400 MHz, DMSO): δ 2.45 (m, 1H), 2.55 (m, 1H), 4.05 (s, 2H), 5.05 (m, 1H), 5.90 (m, 1H), 6.30 (m, 1H), 6.50 (m, 1H), 7.30 (bs, 2H), 8.10 (s, 1H), 8.20 (s, 1H), 8.95 (s, 1H), 10.70 (s, 1H).

### (1R,3R)-1-(9-Adenenyl)-3-carboxymethoxy-4-cyclopentene (18)

Compound 18 was prepared by subjecting compound 16 to general procedure C. Yield=70%. TLC: R_{f}=0.07 (CHCl₃/MeOH/H₂O 150/45/5). Purity: >95% (HPLC method C). ¹H NMR (400 MHz, DMSO): δ 2.45 (m, 2H), 4.10 (s, 2H), 5.10 (m, 1H), 5.85 (m, 1H), 6.25 (m, 1H), 6.45 (m, 1H), 7.35 (s, 2H), 8.15 (s, 1H), 8.25 (s, 1H).

### (1S,3S)-1-(9-Adenenyl)-3-(ethyl-carboxymethoxy)cyclopentane (19)

Compound 19 was prepared by subjecting compound 16 to general procedure G. Yield=94%. TLC: R_{f}=0.23 (CHCl₃/MeOH/H₂O 150/45/5). Purity: >95% (HPLC method C). ¹H NMR (400 MHz, DMSO): δ 1.35 (t, 3H), 1.90 (m, 1H), 2.10 (m, 1H), 2.35 (m, 4H), 4.25 (s, 2H), 4.28 (q, 2H), 4.35 (m, 1H), 5.10 (m, 1H), 7.30 (s, 2H), 8.20 (s, 1H), 8.35 (s, 1H).

### (1S,3S)-1-(9-Adenenyl)-3-(N-hydroxycarbamoylmethoxy)cyclopentane (20)

Compound 20 was prepared by subjecting compound 19 to general procedure B. Yield=90%. TLC: R_{f}=0.36 (CHCl₃/MeOH/H₂O 150/45/5). Purity: >95% (HPLC method C). ¹H NMR (400 MHz, DMSO): δ 1.90 (m, 1H), 2.10 (m, 1H), 2.35 (m, 4H), 3.95 (s, 2H), 4.30 (m, 1H), 5.10 (m, 1H), 7.35 (s, 2H), 8.20 (s, 1H), 8.35 (s, 1H), 8.95 (s, 1H), 10.65 (s, 1H).

### (1S,3S)-1-(9-Adenenyl)-3-carboxymethoxycyclopentane (21)

Compound 21 was prepared by subjecting compound 19 to general procedure C. Yield=100%. TLC: R_{f}=0.07 (CHCl₃/MeOH/H₂O 150/45/5). Purity: >95% (HPLC method C). ¹H NMR (400 MHz, DMSO): δ 1.90 (m, 1H), 2.10 (m, 1H), 2.35 (m, 4H), 4.15 (s, 2H), 4.35 (m, 1H), 5.10 (m, 1H), 7.30 (s, 2H), 8.25 (s, 1H), 8.35 (s, 1H).

### (1R,3R)-1-(tert-Butyl-dimethylsiloxy)-3-hydroxy-4-cyclopentene (22)

Compound 14 was subjected to general procedure J. Yield=81%. TLC: R_{f}=0.50 (10% EtOAc/Hexane). ¹H NMR (400 MHz, CDCl₃): δ 0.05 (s, 6H), 0.90 (s, 9H), 2.20 (m, 1H), 2.30 (m, 1H), 5.10 (s, 1H), 6.05 (m, 2H), 6.10 (m, 1H), 8.20 (d, 2H), 8.30 (d, 2H). Subsequent subjection of the product to general procedure E gave compound 22. Yield=85%. TLC: R_{f}=0.33 (25% EtOAc/Hexane). ¹H NMR (400 MHz, CDCl₃): δ 0.05 (s, 6H), 0.90 (s, 9H), 2.05 (m, 2H), 5.00 (m, 1H), 5.05 (m, 1H), 5.95 (m, 2H).

### (1S,3S)-1-Hydroxy-3-(ethyl-carboxymethoxy)-4-cyclopentene (23)

Compound 22 was subjected to general procedure D. Yield=71%. TLC: R_{f}=0.33 (10% EtOAc/Hexane). ¹H NMR (400 MHz, CDCl₃): δ 0.05 (s, 6H), 0.90 (s, 9H), 1.30 (t, 3H), 1.90 (m, 1H), 2.20 (m, 1H), 4.05 (s, 2H), 4.20 (q, 2H), 4.75 (m, 1H), 5.05 (m, 1H), 6.00 (m, 2H). Subsequent subjection of the product to general procedure K gave compound 23. Yield=91%. TLC: R_{f}=0.25 (50% EtOAc/Hexane). ¹H NMR (400 MHz, CDCl₃): δ 1.25 (t, 3H), 2.00 (m, 1H), 2.25 (m, 1H), 4.05 (s, 2H), 4.20 (q, 2H), 4.80 (m, 1H), 5.05 (m, 1H), 6.05 (m, 2H).

### (1R, 3S)-1-(9-Adenenyl)-3-(ethyl-carboxymethoxy)-4-cyclopentene (24)

Compound 24 was prepared by subjecting compound 23 to general procedure F. Yield=13%. TLC: R_{f}=0.21 (5% MeOH/CHCl₃). Purity: >95% (HPLC method C). ¹H NMR (400 MHz, DMSO): δ 1.30 (t, 3H), 2.05 (m, 1H), 3.00 (m, 1H), 4.25 (q, 2H), 4.35 (s, 2H), 4.80 (m, 1H), 5.60 (m, 1H), 6.30 (m, 1H), 6.45 (m, 1H), 7.35 (s, 2H), 8.15 (s, 1H), 8.25 (s, 1H).

### (1R,3S)-1-(9-Adenenyl)-3-(N-hydroxycarbamoylmethoxy)-4-cyclopentene (25)

Compound 25 was prepared by subjecting compound 24 to general procedure B. Yield=88%. TLC: R_{f}=0.32 (CHCl₃/MeOH/H₂O 150/45/5). Purity: >90% (HPLC method C). ¹H NMR (400 MHz, DMSO): 6 2.05 (m, 1H), 3.00 (m, 1H), 4.10 (s, 2H), 4.75 (m, 1H), 5.60 (m, 1H), 6.25 (m, 1H), 6.45 (m, 1H), 7.35 (s, 2H), 8.15 (s, 1H), 8.25 (s, 1H), 8.95 (s, 1H), 10.70 (s, 1H).

### (1R,3S)-1-(9-Adenenyl)-3-carboxymethoxy-4-cyclopentene (26)

Compound 26 was prepared by subjecting compound 24 to general procedure C. Yield=100%. TLC: R_{f}=0.07 (CHCl₃/MeOH/H₂O 150/45/5). Purity: >75% (HPLC method C). ¹H NMR (400 MHz, DMSO): δ 2.00 (m, 1H), 2.95 (m, 1H), 3.90 (s, 2H), 4.75 (m, 1H), 5.55 (m, 1H), 6.20 (m, 1H), 6.45 (m, 1H), 7.30 (s, 2H), 8.10 (s, 1H), 8.15 (s, 1H).

### (1S,3R)-1-(9-Adenenyl)-3-(ethyl-carboxymethoxy)cyclopentane (27)

Compound 27 was prepared by subjecting compound 24 to general procedure G. Yield=89%. TLC: R_{f}=0.18 (5% MeOH/CHCl₃). Purity: >95% (HPLC method C). ¹H NMR (400 MHz, DMSO): δ 1.35 (t, 3H), 1.95 (m, 1H), 2.10 (m, 1H), 2.15 (m, 2H), 2.35 (m, 1H), 2.60 (m, 1H), 4.25 (m, 5H), 5.05 (m, 1H), 7.30 (s, 2H), 8.25 (s, 1H), 8.35 (s, 1H).

### (1S,3R)-1-(9-Adenenyl)-3-(N-hydroxycarbamoylmethoxy)cyclopentane (28)

Compound 28 was prepared by subjecting compound 27 to general procedure B. Yield=86%. TLC: R_{f}=0.35 (CHCl₃/MeOH/H₂O 150/45/5). Purity: >95% (HPLC method C). ¹H NMR (400 MHz, DMSO): δ 1.90 (m, 1H), 2.10 (m, 1H), 2.20 (m, 3H), 2.60 (m, 1H), 3.95 (s, 2H), 4.20 (m, 1H), 5.00 (m, 1H), 7.30 (s, 2H), 8.20 (s, 1H), 8.35 (s, 1H), 8.95 (s, 1H), 10.65 (s, 1H).

### (1S,3R)-1-(9-Adenenyl)-3-carboxymethoxycyclopentane (29)

Compound 29 was prepared by subjecting compound 27 to general procedure C. Yield=100%. TLC: R_{f}=0.11 (CHCl₃/MeOH/H₂O 150/45/5). Purity: >90% (HPLC method C). ¹H NMR (400 MHz, DMSO): δ 1.90 (m, 1H), 2.10 (m, 1H), 2.15 (m, 2H), 2.30 (m, 1H), 2.55 (m, 1H), 4.00 (s, 2H), 4.25 (m, 1H), 5.00 (m, 1H), 7.30 (s, 2H), 8.25 (s, 1H), 8.50 (s, 1H).

### (1R,3R)-1-Hydroxy-3-(methyl-carboxymethoxy)-4-cyclopentene (30)

Compound 15 was subjected to general procedure J. Yield=89%. TLC: R_{f}=0.31 (25% EtOAc/Hexane). ¹H NMR (400 MHz, CDCl₃): δ 1.30 (t, 3H), 2.35 (m, 2H), 4.10 (s, 2H), 4.25 (q, 2H), 4.90 (m, 1H), 6.05 (m, 1H), 6.20 (m, 1H), 6.30 (m, 1H), 8.20 (d, 2H), 8.30 (d, 2H). Subsequent subjection of the product to general procedure E gave compound 30. Yield=91%. TLC: R_{f}=0.18 (50% EtOAc/Hexane). ¹H NMR (400 MHz, CDCl₃): δ 2.00 (m, 1H), 2.25 (m, 1H), 3.75 (s, 3H), 4.05 (s, 2H), 4.80 (m, 1H), 5.05 (m, 1H), 6.05 (s, 2H).

### (1S,3R)-1-(9-Adenenyl)-3-(methyl-carboxymethoxy)-4-cyclopentene (31)

Compound 31 was prepared by subjecting compound 30 to general procedure F. Yield=16%. TLC: R_{f}=0.14 (5% MeOH/CHCl₃). Purity: >95% (HPLC method C). ¹H NMR (400 MHz, DMSO): δ 2.05 (m, 1H), 3.00 (m, 1H), 3.75 (s, 3H), 4.35 (s, 2H), 4.80 (m, 1H), 5.60 (m, 1H), 6.30 (m, 1H), 6.45 (m, 1H), 7.35 (s, 2H), 8.15 (s, 1H), 8.25 (s, 1H).

### (1S,3R)-1-(9-Adenenyl)-3-(N-hydroxycarbamoylmethoxy)-4-cyclopentene (32)

Compound 32 was prepared by subjecting compound 31 to general procedure B. Yield=100%. TLC: R_{f}=0.32 (CHCl₃/MeOH/H₂O 150/45/5). Purity: >95% (HPLC method C). ¹H NMR (400 MHz, DMSO): δ 2.05 (m, 1H), 3.00 (m, 1H), 4.10 (s, 2H), 4.75 (m, 1H), 5.60 (m, 1H), 6.25 (m, 1H), 6.45 (m, 1H), 7.35 (s, 2H), 8.15 (s, 1H), 8.25 (s, 1H), 8.95 (s, 1H), 10.70 (s, 1H).

### (1S,3R)-1-(9-Adenenyl)-3-carboxymethoxy-4-cyclopentene (33)

Compound 33 was prepared by subjecting compound 31 to general procedure C. Yield=88%. TLC: R_{f}=0.07 (CHCl₃/MeOH/H₂O 150/45/5). Purity: >95% (HPLC method C). ¹H NMR (400 MHz, DMSO): δ 2.00 (m, 1H), 2.95 (m, 1H), 4.15 (s, 2H), 4.75 (m, 1H), 5.60 (m, 1H), 6.25 (m, 1H), 6.45 (m, 1H), 7.35 (s, 2H), 8.15 (s, 1H), 8.25 (s, 1H).

### (1R,3S)-1-(9-Adenenyl)-3-(methyl-carboxymethoxy)cyclopentane (34)

Compound 34 was prepared by subjecting compound 31 to general procedure G. Yield=95%. TLC: R_{f}=0.24 (5% MeOH/CHCl₃). Purity: >95% (HPLC method C). ¹H NMR (400 MHz, DMSO): δ 1.95 (m, 1H), 2.10 (m, 1H), 2.15 (m, 2H), 2.35 (m, 1H), 2.60 (m, 1H), 3.80 (s, 3H), 4.25 (m, 1H), 4.30 (s, 2H), 5.05 (m, 1H), 7.30 (s, 2H), 8.25 (s, 1H), 8.35 (s, 1H).

### (1R,3S)-1-(9-Adenenyl)-3-(N-hydroxycarbamoylmethoxy)cyclopentane (35)

Compound 35 was prepared by subjecting compound 34 to general procedure B. Yield=87%. TLC: R_{f}=0.34 (CHCl₃/MeOH/H₂O 150/45/5). Purity: >90% (HPLC method C). ¹H NMR (400 MHz, DMSO): δ 1.90 (m, 1H), 2.10 (m, 1H), 2.20 (m, 3H), 2.60 (m, 1H), 3.95 (s, 2H), 4.20 (m, 1H), 5.00 (m, 1H), 7.30 (s, 2H), 8.20 (s, 1H), 8.35 (s, 1H), 9.00 (s, 1H), 10.70 (s, 1H).

### (1R,3S)-1-(9-Adenenyl)-3-carboxymethoxycyclopentane (36)

Compound 36 was prepared by subjecting compound 34 to general procedure C. Yield=95%. TLC: R_{f}=0.08 (CHCl₃/MeOH/H₂O 150/45/5). Purity: >85% (HPLC method C). ¹H NMR (400 MHz, DMSO): δ 1.90 (m, 1H), 2.10 (m, 1H), 2.15 (m, 2H), 2.30 (m, 1H), 2.55 (m, 1H), 3.90 (s, 2H), 4.25 (m, 1H), 5.00 (m, 1H), 7.30 (s, 2H), 8.25 (s, 1H), 8.60 (s, 1H).

### (1S,3R)-1-Hydroxy-3-triphenylmethoxy-4-cyclopentene (37)

Compound 14 was subjected to general procedure O. TLC: R_{f}=0.27 (Hexane). ¹H NMR (400 MHz, CDCl₃): δ 0.00 (s, 6H), 1.85 (s, 9H), 1.55 (m, 1H), 2.15 (m, 1H), 4.35 (m, 2H), 4.95 (m, 1H), 5.60 (m, 1H), 7.20 (m, 3H), 7.25 (m, 6H), 7.50 (d, 6H). Subsequent subjection of the product to general procedure K gave compound 37. Yield=87% (2 steps). TLC: R_{f}=0.32 (25% EtOAc/Hexane). ¹H NMR (400 MHz, CDCl₃): δ 1.40 (m, 1H), 2.20 (m, 1H), 4.35 (m, 1H), 4.45 (m, 1H), 5.15 (m, 1H), 5.75 (m, 1H), 7.20 (m, 3H), 7,25 (t, 6H), 7.50 (d, 6H).

### (1R,3R)-1-Chloro-3-triphenylmethoxy-4-cyclopentene (38)

Compound 38 was prepared by subjecting compound 37 to general procedure L. Yield=84%. TLC: R_{f}=0.61 (10% EtOAc/Hexane). ¹H NMR (400 MHz, CDCl₃): δ 2.00 (m, 1H), 2.15 (m, 1H), 4.95 (m, 2H), 5.15 (m, 1H), 5.80 (m, 1H), 7.20 (m, 3H), 7.25 (t, 6H), 7.50 (d, 6H).

### (1S,3R)-1-(2-Dimethylmalonyl)-3-triphenylmethoxy-4-cyclopentene (39)

Compound 39 was prepared by subjecting compound 38 to general procedure M. Yield=72%. TLC: R_{f}=0.27 (10% EtOAc/Hexane). ¹H NMR (400 MHz, CDCl₃): δ 1.35 (m, 1H), 2.05 (m, 1H), 3.00 (m, 1H), 3.30 (d, 1H), 3.70 (s, 6H), 4.60 (m, 1H), 4.90 (m, 1H), 5.60 (m, 1H), 7.20 (m, 3H), 7.25 (t, 6H), 7.45 (d, 6H).

### (1R,3R)-1-Hydroxy-3-(methyl-carboxymethyl)-4-cyclopentene (40)

Compound 39 was subjected to general procedure N. Yield=80%. TLC: R_{f}=0.45 (10% EtOAc/Hexane). ¹H NMR (400 MHz, CDCl₃): δ 1.30 (m, 1H), 2.10 (m, 1H), 2.35 (m, 1H), 2.45 (m, 1H), 2.75 (m, 1H), 3.65 (s, 3H), 4.60 (m, 1H), 4.85 (m, 1H), 5.60 (m, 1H), 7.20 (m, 3H), 7.25 (t, 6H), 7.45 (d, 6H). Subsequent subjection of the product to general procedure P gave compound 40. Yield=39%. TLC: R_{f}=0.38 (50% EtOAc/Hexane). ¹H NMR (400 MHz, CDCl₃): δ 1.40 (m, 1H), 2.45 (m, 2H), 2.55 (m, 1H), 2.95 (m, 1H), 3.65 (s, 3H), 4.80 (m, 1H), 5.85 (m, 2H).

### (1S,3R)-1-(9-Adenenyl)-3-(methyl-carboxymethyl)-4-cyclopentene (41)

Compound 41 was prepared by subjecting compound 40 to general procedure F. Yield=9%. TLC: R_{f}=0.22 (5% MeOH/CHCl₃). Purity: >85% (HPLC method C). ¹H NMR (400 MHz, DMSO): δ 2.30 (m, 2H), 2.60 (m, 2H), 3.50 (m, 1H), 3.75 (s, 3H), 5.75 (m, 1H), 6.05 (m, 1H), 6.30 (m, 1H), 7.40 (s, 2H), 8.10 (s, 1H), 8.25 (s, 1H).

### (1S,3R)-1-(9-Adenenyl)-3-(N-hydroxycarbamoylmethyl)-4-cyclopentene (42)

Compound 42 was prepared by subjecting compound 41 to general procedure B. Yield=37%. TLC: R_{f}=0.40 (CHCl₃/MeOH/H₂O 150/45/5). Purity: >97% (HPLC method C). 1H NMR (400 MHz, DMSO): δ 2.20 (m, 2H), 2.30 (m, 2H), 5.70 (m, 1H), 6.00 (m, 1H), 6.30 (m, 1H), 7.30 (s, 2H), 8.10 (s, 1H), 8.25 (s, 1H), 8.90 (s, 1H), 10.55 (s, 1H).

### (1S,3R)-1-(9-Adenenyl)-3-carboxymethyl-4-cyclopentene (43)

Compound 43 was prepared by subjecting compound 41 to general procedure C. Yield=97%. TLC: R_{f}=0.42 (CHCl₃/MeOH/H₂O 150/45/5). Purity: >86% (HPLC method C). ¹H NMR (400 MHz, DMSO): δ 2.20-2.40 (m, 4H), 3.45 (m, 1H), 5.70 (m, 1H), 6.00 (m, 1H), 6.30 (m, 1H), 7.30 (s, 2H), 8.10 (s, 1H), 8.20 (s, 1H).

### (1R,3R)-1-(9-Adenenyl)-3-(methyl-carboxymethyl)cyclopentane (44)

Compound 44 was prepared by subjecting compound 41 to general procedure G. Yield=95%. TLC: R_{f}=0.21 (CHCl₃/MeOH/H₂O 150/45/5). Purity: >86% (HPLC method C). 1H NMR (400 MHz, DMSO): δ 1.45 (m, 1H), 1.95 (m, 1H), 2.20 (m, 2H), 2.30 (m, 2H), 2.55 (d, 2H), 2.75 (m, 1H), 3.75 (s, 3H), 5.05 (m, 1H), 7.35 (s, 2H), 8.25 (s, 1H), 8.35 (s, 1H).

### (1R,3R)-1-(9-Adenenyl)-3-(N-hydroxycarbamoylmethyl)cyclopentane (45)

Compound 45 was prepared by subjecting compound 44 to general procedure B. Yield=39%. TLC: R_{f}=0.34 (CHCl₃/MeOH/H₂O 150/45/5). Purity: >99% (HPLC method C). ¹H NMR (400 MHz, DMSO): δ 1.45 (m, 1H), 1.95 (m, 1H), 2.15 (d, 2H), 2.20 (m, 3H), 2.35 (m, 1H), 2.70 (m, 1H), 5.05 (m, 1H), 7.30 (s, 2H), 8.25 (s, 1H), 8.35 (s, 1H), 8.85 (s, 1H), 10.50 (s, 1H).

### (1R,3R)-1-(9-Adenenyl)-3-carboxymethylcyclopentane (46)

Compound 46 was prepared by subjecting compound 44 to general procedure C. Yield=83%. TLC: R_{f}=0.38 (CHCl₃/MeOH/H₂O 150/45/5). Purity: >80% (HPLC method C). ¹H NMR (400 MHz, DMSO): δ 1.45 (m, 1H), 1.95 (m, 1H), 2.15-2.40 (m, 4H), 2.45 (d, 2H), 2.70 (m, 1H), 5.05 (m, 1H), 7.30 (s, 2H), 8.25 (s, 1H), 8.35 (s, 1H).

### (1S,3S)-1-Chloro-3-(tert-Butyl-dimethylsiloxy)-4-cyclopentene (47)

Compound 47 was prepared by subjecting compound 14 to general procedure L. Yield=79%. TLC: R_{f}=0.80 (10% EtOAc/Hexane). ¹H NMR (400 MHz, CDCl₃): δ 0.10 (s, 6H), 0.90 (s, 9H), 2.15 (m, 1H), 2.50 (m, 1H), 5.05 (m, 1H), 5.15 (m, 1H), 5.95 (m, 2H).

### (1R,3S)-1-(2-Dimethylmalonyl)-3-(tert-Butyl-dimethylsiloxy)-4-cyclopentene (48)

Compound 48 was prepared by subjecting compound 47 to general procedure M. Yield=74%. TLC: R_{f}=0.32 (10% EtOAc/Hexane). ¹H NMR (400 MHz, CDCl₃): δ 0.10 (s, 6H), 0.90 (s, 9H), 1.40 (m, 1H), 2.40 (m, 1H), 3.20 (m, 1H), 3.35 (m, 1H), 3.75 (s, 6H), 4.80 (m, 1H), 5.80 (m, 2H).

### (1S,3S)-1-Hydroxy-3-(methyl-carboxymethyl)-4-cyclopentene (49)

Compound 48 was subjected to general procedure N. Yield=75%. TLC: R_{f}=0.57 (10% EtOAc/Hexane). ¹H NMR (400 MHz, CDCl₃): δ 0.05 (s, 6H), 0.90 (s, 9H), 1.30 (m, 1H), 2.35 (m, 1H), 2.45 (m, 2H), 2.90 (m, 1H), 3.65 (s, 3H), 4.80 (m, 1H), 5.75 (m, 1H), 5.80 (m, 1H). Subsequent subjection of the product to general procedure I gave compound 49. Yield=61%. TLC: R_{f}=0.39 (50% EtOAc/Hexane). ¹H NMR (400 MHz, CDCl₃): δ 1.40 (m, 1H), 2.25 (m, 2H), 2.35 (m, 1H), 2.95 (m, 1H), 3.65 (s, 3H), 4.80 (m, 1H), 5.85 (m, 2H).

### (1R,3S)-1-(9-Adenenyl)-3-(methyl-carboxymethyl)-4-cyclopentene (50)

Compound 50 was prepared by subjecting compound 49 to general procedure F. Yield=10%. TLC: R_{f}=0.14 (5% MeOH/CHCl₃). Purity: >85% (HPLC method C). ¹H NMR (400 MHz, DMSO): δ 2.30 (m, 2H), 2.60 (m, 2H), 3.75 (s, 3H), 5.75 (m, 1H), 6.05 (m, 1H), 6.30 (m, 1H), 7.30 (s, 2H), 8.10 (s, 1H), 8.25 (s, 1H).

### (1R,3S)-1-(9-Adenenyl)-3-(N-hydroxycarbamoylmethyl)-4-cyclopentene (51)

Compound 51 was prepared by subjecting compound 50 to general procedure B. Yield=63%. TLC: R_{f}=0.40 (CHCl₃/MeOH/H₂O 150/45/5). Purity: >90% (HPLC method C). 1 H NMR (400 MHz, DMSO): δ 2.20 (m, 2H), 2.30 (m, 2H), 5.70 (m, 1H), 6.00 (m, 1H), 6.30 (m, 1H), 7.30 (s, 2H), 8.10 (s, 1H), 8.25 (s, 1H), 9.10 (s, 1H), 10.60 (s, 1H).

### (1R,3S)-1-(9-Adenenyl)-3-carboxymethyl-4-cyclopentene (52)

Compound 52 was prepared by subjecting compound 50 to general procedure C. Yield=100%. TLC: R_{f}=0.31 (CHCl₃/MeOH/H₂O 150/45/5). Purity: >85% (HPLC method C). ¹H NMR (400 MHz, DMSO): δ 2.20-2.40 (m, 4H), 4.15 (m, 1H), 5.70 (m, 1H), 5.95 (m, 1H), 6.30 (m, 1H), 7.30 (s, 2H), 8.10 (s, 1H), 8.20 (s, 1H).

### (1S,3S)-1-(9-Adenenyl)-3-(methyl-carboxymethyl)cyclopentane (53)

Compound 53 was prepared by subjecting compound 50 to general procedure G. Yield=93%. TLC: R_{f}=0.22 (CHCl₃/MeOH/H₂O 150/45/5). Purity: >93% (HPLC method C). ¹H NMR (400 MHz, DMSO): δ 1.45 (m, 1H), 1.95 (m, 1H), 2.20 (m, 2H), 2.30 (m, 2H), 2.55 (d, 2H), 2.75 (m, 1H), 3.75 (s, 3H), 5.05 (m, 1H), 7.35 (s, 2H), 8.25 (s, 1H), 8.35 (s, 1H).

### (1S,3S)-1-(9-Adenenyl)-3-(N-hydroxycarbamoylmethyl)cyclopentane (54)

Compound 54 was prepared by subjecting compound 53 to general procedure B. Yield=34%. TLC: R_{f}=0.39 (CHCl₃/MeOH/H₂O 150/45/5). Purity: >85% (HPLC method C). ¹H NMR (400 MHz, DMSO): δ 1.45 (m, 1H), 1.95 (m, 1H), 2.15 (d, 2H), 2.20 (m, 3H), 2.35 (m, 1H), 2.70 (m, 1H), 5.05 (m, 1H), 7.30 (s, 2H), 8.15 (s, 1H), 8.35 (s, 1H), 8.85 (s, 1H), 10.50 (s, 1H).

### (1S,3S)-1-(9-Adenenyl)-3-carboxymethylcyclopentane (55)

Compound 55 was prepared by subjecting compound 53 to general procedure C. Yield=83%. TLC: R_{f}=0.33 (CHCl₃/MeOH/H₂O 150/45/5). Purity: >80% (HPLC method C). ¹H NMR (400 MHz, DMSO): δ 1.45 (m, 1H), 1.95 (m, 1H), 2.15-2.40 (m, 4H), 2.45 (d, 2H), 2.70 (m, 1H), 5.05 (m, 1H), 7.30 (s, 2H), 8.25 (s, 1H), 8.35 (s, 1H).

### (1R,3S)-1-Chloro-3-(tert-Butyl-dimethylsiloxy)-4-cyclopentene (56)

Compound 56 was prepared by subjecting compound 22 to general procedure L. Yield=99%. TLC: R_{f}=0.86 (10% EtOAc/Hexane). ¹H NMR (400 MHz, CDCl₃): δ 0.10 (s, 6H), 0.90 (s, 9H), 1.95 (m, 1H), 2.90 (m, 1H), 4.75 (m, 2H), 5.90 (m, 2H).

### (1S,3S)-1-(2-Dimethylmalonyl)-3-(tert-Butyl-dimethylsiloxy)-4-cyclopentene (57)

Compound 57 was prepared by subjecting compound 56 to general procedure M. Yield=75%. TLC: R_{f}=0.36 (10% EtOAc/Hexane). ¹H NMR (400 MHz, CDCl₃): δ 0.05 (s, 6H), 0.90 (s, 9H), 1.95 (m, 2H), 3.20 (d, 1H), 3.55 (m, 1H), 3.75 (s, 6H), 4.90 (m, 1H), 5.80 (m, 2H).

### (1S,3R)-1-Hydroxy-3-(methyl-carboxymethyl)-4-cyclopentene (58)

Compound 57 was subjected to general procedure N. Yield=74%. TLC: R_{f}=0.58 (10% EtOAc/Hexane). ¹H NMR (400 MHz, DMSO): δ 0.20 (s, 6H), 0.95 (s, 9H), 1.80 (m, 1H), 1.95 (m, 1H), 2.40 (m, 1H), 2.45 (m, 1H), 3.20 (m, 1H), 3.70 (s, 3H), 5.00 (m, 1H), 5.85 (m, 1H), 5.95 (m, 1H). Subsequent subjection of the product to general procedure K gave compound 58. Yield=59%. TLC: R_{f}=0.40 (50% EtOAc/Hexane). ¹H NMR (400 MHz, CDCl₃): δ 1.85 (m, 1H), 2.00 (m, 1H), 2.35 (m, 2H), 3.30 (m, 1H), 3.65 (s, 3H), 4.90 (m, 1H), 5.85 (m, 1H), 5.95 (m, 1H).

### (1R,3R)-1-(9-Adenenyl)-3-(methyl-carboxymethyl)-4-cyclopentene (59)

Compound 59 was prepared by subjecting compound 58 to general procedure F. Yield=4.3%. TLC: R_{f}=0.17 (5% MeOH/CHCl₃). Purity: >81% (HPLC method C). ¹H NMR (400 MHz, DMSO): δ 1.75 (m, 1H), 2.62 (m, 1H), 2.75 (m, 1H), 2.95 (m, 1H), 3.25 (m, 1H), 3.75 (s, 3H), 5.70 (m, 1H), 6.05 (m, 1H), 6.20 (m, 1H), 7.35 (s, 2H), 8.20 (s, 1H), 8.25 (s, 1H).

### (1R,3R)-1-(9-Adenenyl)-3-(N-hydroxycarbamoylmethyl)-4-cyclopentene (60) and (1R,3R)-1-(9-Adenenyl)-3-carboxymethyl-4-cyclopentene (61)

Compound 59 was subjected to general procedure B and the products were separated by preparative TLC (CHCl₃/MeOH/H₂O 150/45/5). Hydroxamic acid (60): Yield=21%. TLC: R_{f}=0.29 (CHCl₃/MeOH/H₂O 150/45/5). Purity: >90% (HPLC method C). ¹H NMR (400 MHz, DMSO): δ 1.75 (m, 1H), 2.20 (m, 1H), 2.35 (m, 1H), 2.90 (m, 1H), 3.25 (m, 1H), 5.70 (m, 1H), 6.05 (m, 1H), 6.20 (m, 1H), 7.35 (s, 2H), 8.15 (s, 1H), 8.25 (s, 1H), 9.10 (s, 1H), 10.60 (s, 1H). Carboxylic acid (61): Yield=31%. TLC: R_{f}=0.25 (CHCl₃/MeOH/H₂O 150/45/5). Purity: >99% (HPLC method C). ¹H NMR (400 MHz, DMSO): δ 1.70 (m, 1H), 2.20 (m, 1H), 2.30 (m, 1H), 2.90 (m, 1H), 3.20 (m, 1H), 5.65 (m, 1H), 6.00 (m, 1H), 6.35 (m, 1H), 7.35 (s, 2H), 8.15 (s, 1H), 8.25 (s, 1H).

### (1S,3R)-1-(9-Adenenyl)-3-(methyl-carboxymethyl)cyclopentane (62)

Compound 62 was prepared by subjecting compound 59 to general procedure G. Yield=85%. TLC: R_{f}=0.27 (5% MeOH/CHCl₃). ¹H NMR (400 MHz, DMSO): δ 1.72 (m, 1H), 1.88 (m, 1H), 2.04 (m, 1H), 2.16 (m, 1H), 2.28 (m, 1H), 2.46 (m, 2H), 3.70 (s, 3H), 4.95 (m, 1H), 7.30 (s, 2H), 8.25 (s, 1H), 8.35 (s, 1H).

### (1S,3R)-1-(9-Adenenyl)-3-(N-hydroxycarbamoylmethyl)cyclopentane (63) and (1S,3R)-1-(9-Adenenyl)-3-carboxymethylcyclopentane (64)

Compound 62 was subjected to general procedure B and the products were separated by preparative HPLC and isolated as TFA salts. Hydroxamic acid (63): Yield=32%. TLC: R_{f}=0.33 (CHCl₃/MeOH/H₂O 150/45/5). Purity: >90% (HPLC method C). ¹H NMR (400 MHz, DMSO): δ 1.70 (m, 1H), 1.85 (m, 1H), 2.00 (m, 1H), 2.20 (m, 1H), 2.25 (m, 2H), 2.30 (m, 1H), 2.45 (m, 2H), 5.00 (m, 1H), 8.45 (s, 1H), 8.55 (s, 1H). Carboxylic acid (64): Yield=11%. TLC: R_{f}=0.33 (CHCl₃/MeOH/H₂O 150/45/5). Purity: >95% (HPLC method C). ¹H NMR (400 MHz, DMSO): δ 1.70 (m, 1H), 1.85 (m, 1H), 2.05 (m, 1H), 2.15 (m, 1H), 2.30 (m, 1H), 2.45 (m, 1H), 2.50 (m, 3H), 5.05 (m, 1H), 8.50 (s, 1H), 8.60 (s, 1H).

### (1R,3R)-1-Hydroxy-3-triphenylmethoxy-4-cyclopentene (65)

Compound 37 was subjected to general procedure J. TLC: R_{f}=0.36 (10% EtOAc/Hexane). ¹H NMR (400 MHz, DMSO): δ 2.00 (m, 1H), 2.15 (m, 1H), 4.95 (m, 1H), 5.40 (m, 1H), 5.95 (m, 1H), 6.05 (m, 1H), 7.30-7.60 (m, 15H), 8.20 (d, 2H), 8.40 (d, 2H). Subsequent subjection of the product to general procedure E gave compound 65. Yield=77% (2 steps). TLC: R_{f}=0.32 (25% EtOAc/Hexane). ¹H NMR (400 MHz, DMSO): δ 1.55 (m, 1H), 1.80 (m, 1H), 4.70 (m, 1H), 4.80 (m, 1H), 5.10 (m, 1H), 5.80 (m, 1H), 7.40 (m, 3H), 7.45 (m, 6H), 7.55 (m, 6H).

### (1S,3R)-1-Chloro-3-triphenylmethoxy-4-cyclopentene (66)

Compound 66 was prepared by subjecting compound 65 to general procedure L. Yield=93%. TLC: R_{f}=0.58 (10% EtOAc/Hexane). ¹H NMR (400 MHz, CDCl₃): δ 1.90 (m, 1H), 2.40 (m, 1H), 4.55 (m, 2H), 5.20 (m, 1H), 5.75 (m, 1H), 7.20 (m, 3H), 7.25 (t, 6H), 7.50 (d, 6H).

### (1R,3R)-1-(2-Dimethylmalonyl)-3-triphenylmethoxy-4-cyclopentene (67)

Compound 67 was prepared by subjecting compound 66 to general procedure M. Yield=74%. TLC: R_{f}=0.22 (10% EtOAc/Hexane). ¹H NMR (400 MHz, DMSO): δ 1.60 (m, 1H), 1.80 (m, 1H), 3.70 (s, 3H), 3.71 (s, 3H), 4.70 (m, 1H), 5.00 (m, 1H), 5.75 (m, 1H), 7.40 (m, 3H), 7.45 (t, 6H), 7.55 (d, 6H).

### (1R,3S)-1-Hydroxy-3-(methyl-carboxymethyl)-4-cyclopentene (68)

Compound 67 was subjected to general procedure N. Yield=63%. TLC: R_{f}=0.45 (10% EtOAc/Hexane). ¹H NMR (400 MHz, CDCl₃): δ 1.45 (m, 1H), 1.90 (m, 1H), 2.15 (m, 2H), 3.15 (m, 1H), 3.60 (s, 3H), 4.70 (m, 1H), 4.80 (m, 1H), 5.70 (m, 1H), 7.20 (m, 3H), 7.25 (t, 6H), 7.45 (d, 6H). Subsequent subjection of the product to general procedure P gave compound 68. Yield=54%. TLC: R_{f}=0.35 (50% EtOAc/Hexane). ¹H NMR (400 MHz, CDCl₃): δ 1.40 (m, 1H), 1.85 (m, 1H), 2.00 (m, 1H), 2.35 (m, 2H), 3.30 (m, 1H), 3.65 (s, 3H), 4.85 (m, 1H), 5.85 (m, 1H), 5.95 (m, 1H).

### (1S,3S)-1-(9-Adenenyl)-3-(methyl-carboxymethyl)-4-cyclopentene (69)

Compound 69 was prepared by subjecting compound 68 to general procedure F. Yield=16%. TLC: R_{f}=0.17 (5% MeOH/CHCl₃). Purity: >87% (HPLC method C). ¹H NMR (400 MHz, DMSO): δ 1.75 (m, 1H), 2.62 (m, 1H), 2.75 (m, 1H), 2.95 (m, 1H), 3.25 (m, 1H), 3.75 (s, 3H), 5.70 (m, 1H), 6.05 (m, 1H), 6.20 (m, 1H), 7.35 (s, 2H), 8.20 (s, 1H), 8.25 (s, 1H).

### (1S,3S)-1-(9-Adenenyl)-3-(N-hydroxycarbamoylmethyl)-4-cyclopentene (70)

Compound 70 was prepared by subjecting compound 69 to general procedure B. Yield=49%. TLC: R_{f}=0.36 (CHCl₃/MeOH/H₂O 150/45/5). Purity: >89% (HPLC method C). ¹H NMR (400 MHz, DMSO): δ 1.75 (m, 1H), 2.20 (m, 1H), 2.35 (m, 1H), 2.90 (m, 1H), 3.25 (m, 1H), 5.70 (m, 1H), 6.05 (m, 1H), 6.20 (m, 1H), 7.35 (s, 2H), 8.15 (s, 1H), 8.25 (s, 1H), 8.90 (s, 1H), 10.50 (s, 1H).

### (1S,3S)-1-(9-Adenenyl)-3-carboxymethyl-4-cyclopentene (71)

Compound 71 was prepared by subjecting compound 69 to general procedure C. Yield=75%. TLC: R_{f}=0.34 (CHCl₃/MeOH/H₂O 150/45/5). Purity: >84% (HPLC method C). ¹H NMR (400 MHz, DMSO): δ 1.70 (m, 1H), 2.30 (m, 1H), 2.45 (m, 1H), 2.90 (m, 1H), 3.20 (m, 1H), 5.65 (m, 1H), 6.00 (m, 1H), 6.25 (m, 1H), 7.35 (s, 2H), 8.15 (s, 1H), 8.25 (s, 1H).

### (1R,3S)-1-(9-Adenenyl)-3-(methyl-carboxymethyl)cyclopentane (72)

Compound 72 was prepared by subjecting compound 69 to general procedure G. Yield=92%. TLC: R_{f}=0.27 (5% MeOH/CHCl₃). Purity: >87% (HPLC method C). ¹H NMR (400 MHz, DMSO): δ 1.72 (m, 1H), 1.88 (m, 1H), 2.04 (m, 1H), 2.16 (m, 1H), 2.28 (m, 1H), 2.46 (m, 2H), 2.62 (m, 2H), 3.70 (s, 3H), 4.95 (m, 1H), 7.30 (s, 2H), 8.25 (s, 1H), 8.35 (s, 1H).

### (1R,3S)-1-(9-Adenenyl)-3-(N-hydroxycarbamoylmethyl)cyclopentane (73)

Compound 73 was prepared by subjecting compound 72 to general procedure B. Yield=56%. TLC: R_{f}=0.42 (CHCl₃/MeOH/H₂O 150/45/5). Purity: >94% (HPLC method C). ¹H NMR (400 MHz, DMSO): δ 1.70 (m, 1H), 1.85 (m, 1H), 2.00 (m, 1H), 2.20 (m, 1H), 2.25 (m, 2H), 2.30 (m, 1H), 2.45 (m, 2H), 4.95 (m, 1H), 7.30 (s, 2H), 8.25 (s, 1H), 8.35 (s, 1H), 8.85 (bs, 1H), 10.50 (bs, 1H).

### (1R,3S)-1-(9-Adenenyl)-3-carboxymethylcyclopentane (74)

Compound 74 was prepared by subjecting compound 72 to general procedure C. Yield=99%. TLC: R_{f}=0.42 (CHCl₃/MeOH/H₂O 150/45/5). Purity: >82% (HPLC method C). ¹H NMR (400 MHz, DMSO): δ 1.70 (m, 1H), 1.85 (m, 1H), 2.05 (m, 1H), 2.15 (m, 1H), 2.30 (m, 1H), 2.45 (m, 4H), 4.95 (m, 1H), 7.35 (s, 2H), 8.20 (s, 1H), 8.35 (s, 1H).

### (1S,3R)-Methyl-1-aminocyclopent-4-ene-3-carboxylate hydrochloride (76a)

Compound 76a was prepared by subjecting (1S,3R)-1-aminocyclopent-4-ene-3-carboxylic acid to general procedure Q. Yield=100%. ¹H NMR (400 MHz, DMSO): δ 2.05 (m, 1H), 2.65 (m, 1H), 3.80 (s, 3H), 3.85 (m, 1H), 4.30 (m, 1H), 6.00 (m, 1H), 6.20 (m, 1H), 8.40 (bs, 3H).

### (1R,3S)-Methyl-1-aminocyclopent-4-ene-3-carboxylate hydrochloride (76b)

Compound 76b was prepared by subjecting (1R,3S)-1-aminocyclopent-4-ene-3-carboxylic acid to general procedure Q. Yield=100%. ¹H NMR (400 MHz, DMSO): δ 2.05 (m 1H), 2.65 (m, 1H), 3.80 (s, 3H), 3.85 (m, 1H), 4.30 (m, 1H), 6.00 (m, 1H), 6.20 (m, 1H), 8.40 (bs, 3H).

### (1S,3R)-1-[9-(1-Chloroadenenyl)]-3-methylcarboxy-4-cyclopentene (77a)

Compound 76a was subjected to general procedure R. Subsequent subjection of the crude product to general procedure S yielded the desired crude aminopyrimidine. Without purification, the crude aminopyrimidine was subjected to general procedure T giving compound 77a. Yield=40% (3 steps). TLC: R_{f}=0.50% (EtOAc/Hexane). Purity: >90% (HPLC method C). ¹H NMR (400 MHz, DMSO): δ 2.35 (m, 1H), 3.00 (m, 1H), 3.80 (s, 3H), 3.95 (m, 1H), 5.90 (m, 1H), 6.25 (m, 1H), 6.40 (m, 1H), 8.65 (s, 1H), 8.90 (s, 1H).

### (1R,3S)-1-[9-(1-Chloroadenenyl)]-3-methylcarboxy-4-cyclopentene (77b)

Compound 76b was subjected to general procedure R. Subsequent subjection of the crude product to general procedure S yielded the desired crude aminopyrimidine. Without purification, the crude aminopyrimidine was subjected to general procedure T giving compound 77b. Yield=38% (3 steps). TLC: R_{f}=0.50% (EtOAc/Hexane). Purity: >90% (HPLC method C). ¹H NMR (400 MHz, DMSO): δ 2.35 (m, 1H), 3.00 (m, 1H), 3.80 (s, 3H), 3.95 (m, 1H), 5.90 (m, 1H), 6.25 (m, 1H), 6.40 (m, 1H), 8.65 (s, 1H), 8.90 (s, 1H).

### (1S,3R)-1-[9-(1-Azidoadenenyl)]-3-methylcarboxy-4-cyclopentene (78a)

Compound 78a was prepared by subjecting compound 77a to general procedure U. Yield=52%. Purity: >99% (HPLC method C). ¹H NMR (400 MHz, DMSO): δ 2.40 (m, 1H), 3.05 (m, 1H), 3.80 (s, 3H), 4.00 (m, 1H), 6.00 (m, 1H), 6.30 (m, 1H), 6.45 (m, 1H), 8.60 (m, 1H), 10.25 (s, 1H).

### (1R,3S)-1-[9-(1-Azidoadenenyl)]-3-methylcarboxy-4-cyclopentene (78b)

Compound 78b was prepared by subjecting compound 77b to general procedure U. Yield=60%. Purity: >99% (HPLC method C). ¹H NMR (400 MHz, DMSO): δ 2.40 (m, 1H), 3.05 (m, 1H), 3.80 (s, 3H), 4.00 (m, 1H), 6.00 (m, 1H), 6.30 (m, 1H), 6.45 (m, 1H), 8.60 (m, 1H), 10.25 (s, 1H).

### (1R,3S)-1-(9-Adenenyl)-3-methylcarboxycyclopentane (79a)

Compound 79a was prepared by subjecting compound 78a to general procedure G. Yield=99%. Purity: >99% (HPLC method C). ¹H NMR (400 MHz, DMSO): δ 2.20 (m, 3H), 2.30 (m, 1H), 2.40 (m, 1H), 2.55 (m, 1H), 3.15 (m, 1H), 3.80 (s, 3H), 5.00 (m, 1H), 7.35 (s, 2H), 8.20 (m, 1H), 8.35 (s, 1H).

### (1S,3R)-1-(9-Adenenyl)-3-methylcarboxycyclopentane (79b)

Compound 79b was prepared by subjecting compound 78b to general procedure G. Yield=96%. Purity: >99% (HPLC method C). ¹H NMR (400 MHz, DMSO): δ 2.20 (m, 3H), 2.30 (m, 1H), 2.40 (m, 1H), 2.55 (m, 1H), 3.15 (m, 1H), 3.80 (s, 3H), 5.00 (m, 1H), 7.35 (s, 2H), 8.20 (m, 1H), 8.35 (s, 1H).

### (1R,3S)-1-(9-Adenenyl)-3-(N-hydroxycarbamoyl)cyclopentane (80a)

Compound 80a was prepared by subjecting compound 79a to general procedure B. Yield=53%. Purity: >95% (HPLC method C). ¹H NMR (400 MHz, DMSO): δ 2.00 (m, 2H), 2.30 (m, 3H), 2.50 (m, 1H), 2.80 (m, 1H), 5.00 (m, 1H), 7.35 (s, 2H), 8.25 (m, 1H), 8.45 (s, 1H), 8.95 (s, 1H), 10.65 (s, 1H).

### (1S,3R)-1-(9-Adenenyl)-3-(N-hydroxycarbamoyl)cyclopentane (80b)

Compound 80b was prepared by subjecting compound 79b to general procedure B. Yield=53%. Purity: >95% (HPLC method C). ¹H NMR (400 MHz, DMSO): δ 2.00 (m, 2H), 2.30 (m, 3H), 2.50 (m, 1H), 2.80 (m, 1H), 5.00 (m, 1H), 7.35 (s, 2H), 8.25 (m, 1H), 8.45 (s, 1H), 8.95 (s, 1H), 10.65 (s, 1H).

### (1R,3S)-1-(9-Adenenyl)-3-carboxycyclopentane (81 a)

Compound 81a was prepared by subjecting compound 79a to general procedure C. Yield=60%. Purity: >99% (HPLC method C). ¹H NMR (400 MHz, DMSO): δ 2.10-2.40 (m, 5H), 2.55 (m, 1H), 3.05 (m, 1H), 5.00 (m, 1H), 7.35 (s, 2H), 8.25 (s, 1H), 8.35 (s, 1H), 12.40 (s, 1H).

### (1S,3R)-1-(9-Adenenyl)-3-carboxycyclopentane (81b)

Compound 81b was prepared by subjecting compound 79b to general procedure C. Yield=58%. Purity: >99% (HPLC method C). ¹H NMR (400 MHz, DMSO): δ 2.10-2.40 (m, 5H), 2.55 (m, 1H), 3.05 (m, 1H), 5.00 (m, 1H), 7.35 (s, 2H), 8.25 (s, 1H), 8.35 (s, 1H), 12.40 (s, 1H).

### (1S,3R)-1-(9-Adenenyl)-3-methylcarboxy-4-cyclopentene (82a)

Compound 82a was prepared by subjecting compound 78a to general procedure V. Yield=98%. Purity: >99% (HPLC method C). ¹H NMR (400 MHz, DMSO): δ 2.25 (m, 1H), 2.95 (m, 1H), 3.75 (s, 3H), 3.90 (m, 1H), 5.75 (m, 1H), 6.20 (m, 1H); 6.30 (m, 1H), 7.35 (s, 2H), 8.05 (s, 1H), 8.25 (s, 1H).

### (1R,3S)-1-(9-Adenenyl)-3-methylcarboxy-4-cyclopentene (82b)

Compound 82b was prepared by subjecting compound 78b to general procedure V. Yield=97%. Purity: >99% (HPLC method C). ¹H NMR (400 MHz, DMSO): δ 2.25 (m, 1H), 2.95 (m, 1H), 3.75 (s, 3H), 3.90 (m, 1H), 5.75 (m, 1H), 6.20 (m, 1H), 6.30 (m, 1H), 7.35 (s, 2H), 8.05 (s, 1H), 8.25 (s, 1H).

### (1S,3R)-1-(9-Adenenyl)-3-(N-hydroxycarbamoyl)-4-cyclopentene (83a) and (1S,3S)-1-(9-Adenenyl)-3-(N-hydroxycarbamoyl)-4-cyclopentene (84a)

Compound 82a was subjected to general procedure B and the products were separated by preparative HPLC as described. The isolated TFA salts were converted to free bases utilizing MP-carbonate resin (Argonaut) in MeOH. Compound 83a: Yield=44%. TLC: R_{f}=0.34 (CHCl₃/MeOH/H₂O 150/45/5). Purity: >99% (HPLC method C). ¹H NMR (400 MHz, DMSO): δ 2.10 (m, 1H), 2.90 (m, 1H), 3.55 (m, 1H), 5.80 (m, 1H), 6.15 (m, 1H), 6.20 (m, 1H), 7.35 (s, 2H), 8.25 (s, 1H), 8.35 (s, 1H), 9.05 (bs, 1H), 10.80 (bs, 1H). Compound 84a: Yield=26%. TLC: R_{f}=0.29 (CHCl₃/MeOH/H₂O 150/45/5). Purity: >99% (HPLC method C). ¹H NMR (400 MHz, DMSO): δ 2.30 (m, 1H), 2.75 (m, 1H), 3.85 (m, 1H), 5.85 (m, 1H), 6.15 (m, 1H), 6.20 (m, 1H), 7.35 (s, 2H), 8.15 (s, 1H), 8.25 (s, 1H), 9.00 (s, 1H), 10.80 (s, 1H).

### (1R,3S)-1-(9-Adenenyl)-3-(N-hydroxycarbamoyl)-4-cyclopentene (83b) and (1R,3R)-1-(9-Adenenyl)-3-(N-hydroxycarbamoyl)-4-cyclopentene (84b)

Compound 82b was subjected to general procedure B and the products were separated by preparative HPLC as described. The isolated TFA salts were converted to free bases utilizing MP-carbonate resin (Argonaut) in MeOH. Compound 83b: Yield=44%. TLC: R_{f}=0.32 (CHCl₃/MeOH/H₂O 150/45/5). Purity: >99% (HPLC method C). ¹H NMR (400 MHz, DMSO): δ 2.10 (m, 1H), 2.90 (m, 1H), 3.55 (m, 1H), 5.80 (m, 1H), 6.15 (m, 1H), 6.20 (m, 1H), 7.35 (s, 2H), 8.25 (s, 1H), 8.35 (s, 1H), 9.05 (bs, 1H), 10.80 (bs, 1H). Compound 84b: Yield=24%. TLC: R_{f}=0.26 (CHCl₃/MeOH/H₂O 150/45/5). Purity: >99% (HPLC method C). ¹H NMR (400 MHz, DMSO): δ 2.30 (m, 1H), 2.75 (m, 1H), 3.85 (m, 1H), 5.85 (m, lH), 6.15 (m, 1H), 6.20 (m, 1H), 7.35 (s, 2H), 8.15 (s, 1H), 8.25 (s, 1H), 9.00 (bs, 1H), 10.80 (bs, 1H).

### (1R,3R)-1-(9-Adenenyl)-3-(N-hydroxycarbamoyl)cyclopentane (85a)

Compound 85a was prepared by subjecting compound 84a to general procedure G where 10% Pd/C was replaced with 20% Pd(OH)₂/C. Yield=99%. TLC: R_{f}=0.27 (CHCl₃/MeOH/H₂O 150/45/5). Purity: >97% (HPLC method C). ¹H NMR (400 MHz, DMSO): δ 1.90 (m, 1H), 2.15-2.40 (m, 5H), 2.95 (m, 1H), 5.05 (m, 1H), 7.30 (s, 2H), 8.25 (s, 1H), 8.30 (s, 1H), 8.90 (s, 1H), 10.60 (s, 1H).

### (1S,3S)-1-(9-Adenenyl)-3-(N-hydroxycarbamoyl)cyclopentane (85b)

Compound 85b was prepared by subjecting compound 84b to general procedure G where 10% Pd/C was replaced with 20% Pd(OH)₂/C. Yield=95%. TLC: R_{f}=0.27 (CHCl₃/MeOH/H₂O 150/45/5). Purity: >97% (HPLC method C). ¹H NMR (400 MHz, DMSO): δ 1.90 (m, 1H), 2.15-2.40 (m, 5H), 2.95 (m, 1H), 5.05 (m, 1H), 7.30 (s, 2H), 8.25 (s, 1H), 8.30 (s, 1H), 8.90 (s, 1H), 10.60 (s, 1H).

### (1S,3R)-1-(9-Adenenyl)-3-carboxy-4-cyclopentene (86a) and (1S,3S)-1-(9-Adenenyl)-3-carboxy-4-cyclopentene (87a) and (1R)-1-(9-Adenenyl)-3-carboxy-3-cyclopentene (88a)

Compound 82a was subjected to general procedure C yielding a mixture of compounds 86a, 87a and 88a. Utilizing preparative HPLC (0-10% CH₃CN/30 minutes), compound 86a was separated from compounds 87a and 88a. Compounds 87a and 88a could not be separated from one another. All compounds were isolated as TFA salts. Compound 86a: Yield=30%. TLC: R_{f}=0.19 (CHCl₃/MeOH/H₂O 150/45/5). Purity: >84% (HPLC method C). ¹H NMR (400 MHz, DMSO): δ 2.25 (m, 1H), 2.95 (m, 1H), 3.85 (m, 1H), 5.80 (m, 1H), 6.20 (m, 1H), 6.40 (m, 1H), 8.30 (s, 1H), 8.50 (m, 3H). Compounds 87a and 88a: Yield=60%. 87a/88a=4/5. TLC: R_{f}=0.19 (CHCl₃/MeOH/H₂O 150/45/5). Purity: >99% (HPLC method C). ¹H NMR (400 MHz, DMSO): δ 2.35 (m, 1H, 87a), 2.85 (m, 1H, 87a), 3.05 (m, 2H, 88a), 3.25 (m, 2H, 88a), 4.15 (m, 1H, 87a), 5.45 (m, 1H, 88a), 5.90 (m, 1H, 87a), 6.15 (m, 1H, 87a), 6.35 (m, 1H, 87a), 6.90 (m, 1H, 88a), 8.40 (s, 1H, 87a), 8.50 (s, 1H, 87a), 8.50 (s, 1H, 88a), 8.55 (s, 1H, 88a), 8.60 (bs, 2H, 87a), 8.60 (bs, 2H, 88a).

### (1R,3S)-1-(9-Adenenyl)-3-carboxy-4-cyclopentene (86b) and (1R,3R)-1-(9-Adenenyl)-3-carboxy-4-cyclopentene (87b) and (1S)-1-(9-Adenenyl)-3-carboxy-3-cyclopentene (88b)

Compound 82b was subjected to general procedure C yielding a mixture of compounds 86b, 87b and 88b. Utilizing preparative HPLC (0-10% CH₃CN/30 minutes), compound 86b was separated from compounds 87b and 88b. Compounds 87b and 88b could not be separated from one another. All compounds were isolated as TFA salts. Compound 86b: Yield=41%. TLC: R_{f}=0.20 (CHCl₃/MeOH/H₂O 150/45/5). Purity: >93% (HPLC method C). ¹H NMR (400 MHz, DMSO): δ 2.25 (m, 1H), 2.95 (m, 1H), 3.85 (m, 1H), 5.80 (m, 1H), 6.20 (m, 1H), 6.40 (m, 1H), 8.30 (s, 1H), 8.50 (s, 1H), 8.85 (bs, 2H). Compounds 87b and 88b: Yield=68%. 87b/88b=1/2. TLC: R_{f}=0.20 (CHCl₃/MeOH/H₂O 150/45/5). Purity: >99% (HPLC method C). ¹H NMR (400 MHz, DMSO): δ 2.35 (m, 1H, 87b), 2.85 (m, 1H, 87b), 3.05 (m, 2H, 88b), 3.25 (m, 2H, 88b), 4.15 (m, 1H, 87b), 5.45 (m, 1H, 88b), 5.90 (m, 1H, 87b), 6.15 (m, 1H, 87b), 6.35 (m, 1H, 87b), 6.90 (m, 1H, 88b), 8.40 (s, 1H, 87b), 8.50 (s, 1H, 87b), 8.50 (s, 1H, 88b), 8.55 (s, 1H, 88b), 8.60 (bs, 2H, 87b), 8.60 (bs, 2H, 88b).

The following additional examples demonstrate preferred embodiments of the invention. For the purpose of carrying out the experiments described below aimed at examining the specific role of type 5 AC in regulating cardiac and striatum-mediated motor function, a mouse line was developed in which the type 5 AC gene was disrupted. With regard to cardiac function, the specific questions addressed were whether elimination of type 5 AC (a) decreases either baseline cardiac function or HR, (b) impairs sympathetic stimulation or (c) alters parasympathetic modulation of cardiac function and HR. These questions were addressed using a combination of in vitro and in vivo approaches, e.g., by measuring cardiac function echocardiographically and HR in conscious mice, and assessing AC activity in vitro in cardiac membranes. Specifically, the effect of β-AR stimulation with ISO and muscarinic stimulation with acetylcholine (Ach), both under baseline conditions and also superimposed on β-AR stimulation was also examined. Parasympathetic neural function using intravenously (i.v.) administered phenylephrine to elicit baroreflex mediated slowing of HR, which is known to be predominantly a parasympathetic function were also examined. The protocol for the AC5KO mouse model and discusssion of results are as follows.

### EXAMPLE 2

Generation of Knockout Mice

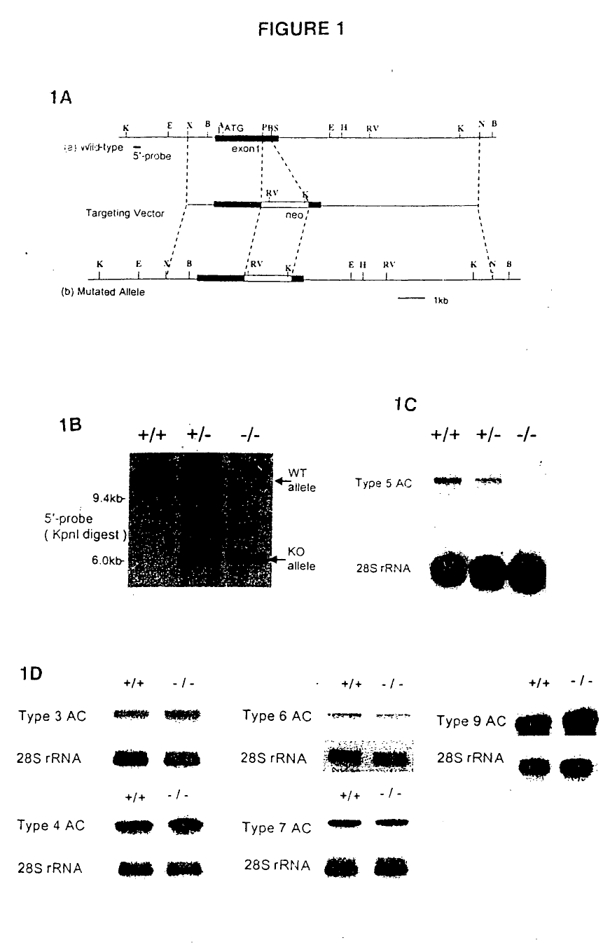

The targeting construct was prepared by ligating a 2.2-kb XhoI-PstI fragment from the 5′ end of the type 5 AC gene, containing the exon with the first translation initiation site (5′-arm), a 1.7-kb fragment containing a neomycin resistance gene fragment (neo) driven by a phosphoglycerate kinase (PGK) promoter, and a BssHII-NcoI 7.0-kb fragment of the type 5 AC gene (3′-arm), into pBluscript II KS (Stratagene, La Jolla, Calif., USA). The type 5 AC gene has another translational start site accompanied by a reasonable Kozak consensus sequence located 738-bp downstream of the first translational start site within the same exon. To impair the second site, inventors excised a 0.15 kb PstI-BssHII fragment containing the second ATG and replaced it with a PGK-neo cassette in the final targeting vector (FIG. 1A).

Embryonic stem cells were transfected with 50 μg linearized targeting vector by electroporation (Bio-Rad Gene pulsar set at 250 V and 960° F.). G418 (200 μg/ml) selection was applied 48 hours after transfection and resistant clones were isolated after 7-10 days of transfection. Subsequently, inventors obtained 576 clones. Genomic DNA from these resistant clones was digested with KpnI and probed with a 5′ probe. Digesting genomic DNA with BamHI and probing with a 3′ probe reconfirmed 8 positive clones. A single integration of the targeting vector was confirmed by a neo-probe. Two clones (clones #314 and #378) were injected into C57BL/6 blastocysts and chimeras were obtained. These chimeras successfully allowed germ-line transmission and were crossed with C57BL/6 females. F1-heterozygous offspring were then interbred to produce homozygous mutations. All mice were 129/SvJ-C57BL/6 mixed background litter mates from F1 heterozygote crosses. All experiments were performed in 4-6 month old homozygous AC5KO and wild-type (WT) littermates.

Rotor Rod Test

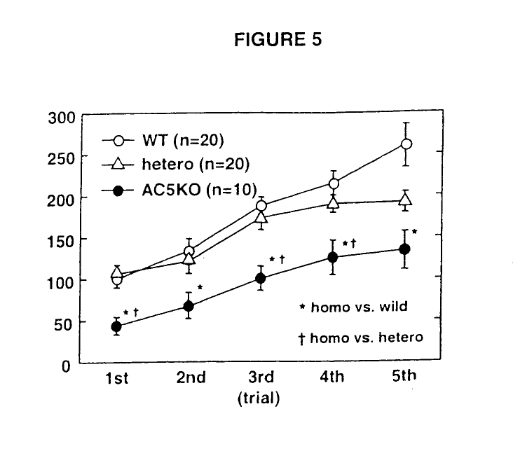

The locomotor activity of intact animals, AC5KO versus WT was examined (FIG. 5). At first glance the animals appeared normal, being neither catatonic nor rigid. However, standard behavior tests revealed that the mice had a significatnt impairment in motor function. The mice were studied using a rotor rod test in which mice were placed on a rototating rod and had to make continuous adjustment in balance in order to remain upright. The time that the mice spent on the accelerating rotor rod without falling was measured. The rod increased from 3 rpm to 30 rpm during each 5 min. trial. Each mouse went through 5 trials, which showed a gradual increase in the time on a rod showing “learning effects”. There was no significant difference between WT and Hetero at the 1^{st} through 4^{th} trial. At the 5^{th} trial, there was a small but signiifcant decrease in their performance in Hetero. AC5KO, by contrast, showed a significant improvement at the 1^{st} trial and constantly had and constantly has a shorter time on a rotor rod with poor learning effect, suggesting that the locomotor activity in AC5KO was significantly impaired.

RNase Protection Assay

Partial fragments of mouse AC cDNA clones for each isoform (types 1-9) were obtained by PCR. Sequencing and restriction mapping verified these cDNA fragments. Total RNA was isolated using RNeasy Midi kit (QIAGEN, Valencia, Calif., USA). Single strand cDNA was synthesized from total RNA using reverse transcriptase. The plasmid constructs were linearized by appropriate restriction enzyme. ³²P-labeled cRNA probes were then generated using the Riboprobe Systems (Promega, Madison, Wis., USA). A human 28S ribosomal RNA probe was used as an internal control. RNase protection assay was performed using the RPA III kit (Ambion, Austin, Tex., USA) as suggested by the manufacture, followed by analysis on a 5% polyacrylamide-urea gel. Gels were exposed to X-OMAT film (Kodak, Rochester, N.Y., USA) for quantitation.

AC Assay and Tissue cAMP Measurement

Hearts were dissected from the mice and membrane preparations were prepared as described previously. Protein concentration was measured by the method of Bradford using bovine serum albumin as a standard. AC activity was measured as described previously. AC activity was linear within the incubation time up to 30 min. In order to harvest hearts for tissue cAMP content measurements, mice were allowed to acclimate to the surroundings in the laboratory for an hour before sacrifice. Freshly isolated hearts were briefly immersed in liquid nitrogen. The tissue was homogenized in ice-cold 6% percholic acid, and cAMP was extracted as described before. The concentration of cAMP was determined with an RIA kit (PerkinElmer Life Sciences, Boston, Mass., USA).

Physiological Studies

AC5KO (6.4±0.2 month old, n=6) and WT (6.7±0.1 month old, n=6) of either sex from the same genetic background as the transgenic mice were used for the physiological studies. Measurements of LV ejection fraction (LVEF) were performed as described previously. Briefly, after determination of body weight, mice were anesthetized with ketamine (0.065 mg/g), acepromazine (0.002 mg/g), and xylazine (0.013 mg/g) injected intraperitoneally and were allowed to breathe spontaneously. Echocardiography was performed using ultrasonography (Sequoia C256; Acuson Corporation, Mountain View, Calif., USA). A dynamically focused 15-MHz annular array transducer was applied from below, using a warmed saline bag as a standoff. M-mode echocardiographic measurements of the LV were performed at baseline and during intravenous infusion of ISO (0.005, 0.01, 0.02, and 0.04 μg/kg/min i.v. for 5 minutes each) (Abbott Laboratories Inc, North Chicago, Ill., USA) using an infusion pump (PHD 2000; Harvard Apparatus, Inc., Holliston, Mass., USA). The total amount of the infusion volume was <100 μL in each mouse. On a separate occasion, each mouse received an infusion of saline as a control to ensure that the volume of infusion alone did not contribute to enhance ventricular performance. To examine the responses to a muscarinic agonist, intraperitoneal (i.p.) infusion of Ach (25 mg/kg) was performed on top of the i.v. infusion of ISO (0.04 μg/kg/min).

In AC5KO and WT mice, four ECG wires (New England Electric Wire Corporation, Lisbon, N.H., USA) were placed subcutaneously, a silicone elastomer tubing (Cardiovascular Instrument Corp., Wakefield, Mass., USA) was inserted into the right external jugular vein and a 1.4 F micromanometer catheter (Millar Instruments, Inc., Houston, Tex., USA) was inserted into the lower abdominal aorta via the femoral artery as described previously with some modifications. The ECG wires, the silicone elastomer tubing and the micromanometer catheter were tunneled subcutaneously to the back, externalized, and secured in a plastic cap. On the day of the study, each mouse was placed in the mouse holder, the jugular venous catheter was accessed and connected to a microliter syringe (Hamilton Co., Reno, Nev., USA), the 1.4 F micromanometer catheter was connected to a recorder (Dash 4u; Astro-Med, Inc., West Warwick, R.I., USA) and the ECG wires were connected to an ECG amplifier (Gould Inc., Cleveland, Ohio, USA). All experiments were recorded with animals in the conscious state. After at least 6 hours recovery from the implantation of the catheter, when a stable HR was achieved, the baseline ECG and arterial pressure (AP) were recorded for 5 min. Ach (0.05 μg/g) was then administered intravenously (i.v.), and the ECG and AP recording were repeated. A recovery period of 15 min was allowed for the HR and AP to return to baseline before administering the next drug. Baseline HR slowing was examined in response to phenylephrine (0.2 μg/g i.v.).

Statistics

All data are reported as mean±SEM. Comparisons between AC5KO and WT values were made using a t-test. P<0.05 was taken as a minimal level of significance.

Results:

Targeted Disruption of the Type 5 AC Gene.

The type 5 AC gene was disrupted in mice using homologous recombination (FIG. 1A). Mice were genotyped by Southern blotting using genomic DNA from tail biopsies (FIG. 1B). mRNA expression of the type 5 AC in heterozygous mice was approximately half of that in WT and it was undetectable in AC5KO (FIG. 1C). The growth, general appearance and behavior were similar to those of WT.

No Compensatory Increase in the Other Isoforms of AC.

Inventors then examined whether there were compensatory increases in the expression of the other isoforms of AC in AC5KO. Since AC isoform antibodies that can convincingly determine the level of protein expression of all the isoforms are not available, inventors quantitated the mRNA expression of the AC isoforms by an RNase protection assay. cRNA of the 28S ribosomal RNA was used as an internal control. Types 3, 4, 6, 7 and 9 AC were readily detected, but not increased (FIG. 1D), while types 1, 2, and 8 were hardly detectable (data not shown), arguing that type 6 AC, a homologue of type 5 AC in the heart, could not compensate for the type 5 AC deficiency. AC activity was decreased in the hearts of AC5KO in vitro.

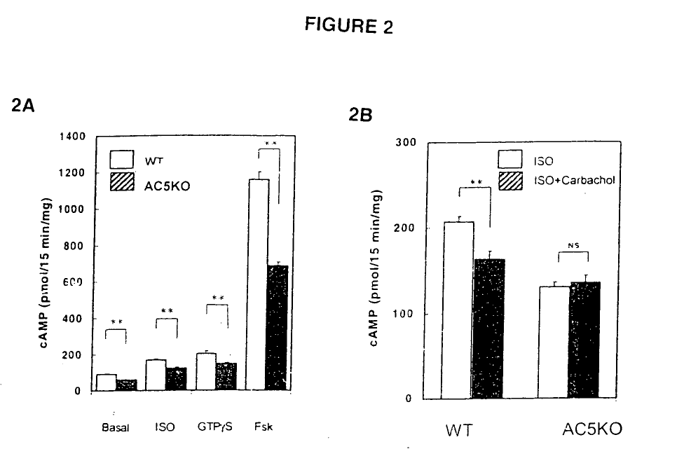

Inventors then examined cAMP production in membranes from the hearts of AC5KO and WT at 6 month of age (FIG. 2A). The steady state AC activity was determined as the maximal capacity of cAMP production in the presence of ISO (100 μM ISO+100 μM GTP), GTPγS (100 μM) or forskolin (100 μM). AC activity was decreased in AC5KO relative to that in WT by 35±4.3% (basal), 27±4.6% (ISO), 27±2.4% (GTPγS), and 40±4.7% (forskolin). These data indicate that type 5 AC, as the major isoform in the heart, is responsible for approximately 30-40% of total AC activity in the mouse heart. However, cardiac tissue cAMP content was not significantly decreased in AC5KO compared to WT (55±7.5 vs 62±3.4 pmol/mg protein, respectively, n=4, p=NS). Carbachol (10 μM), a muscarinic agonist, decreased ISO-stimulated AC activity by 21±3.4% in WT, but did not inhibit ISO-stimulated AC activity in AC5KO (FIG. 2B). Basal cardiac function was not decreased, but the response to ISO and muscarinic inhibition of ISO were attenuated.

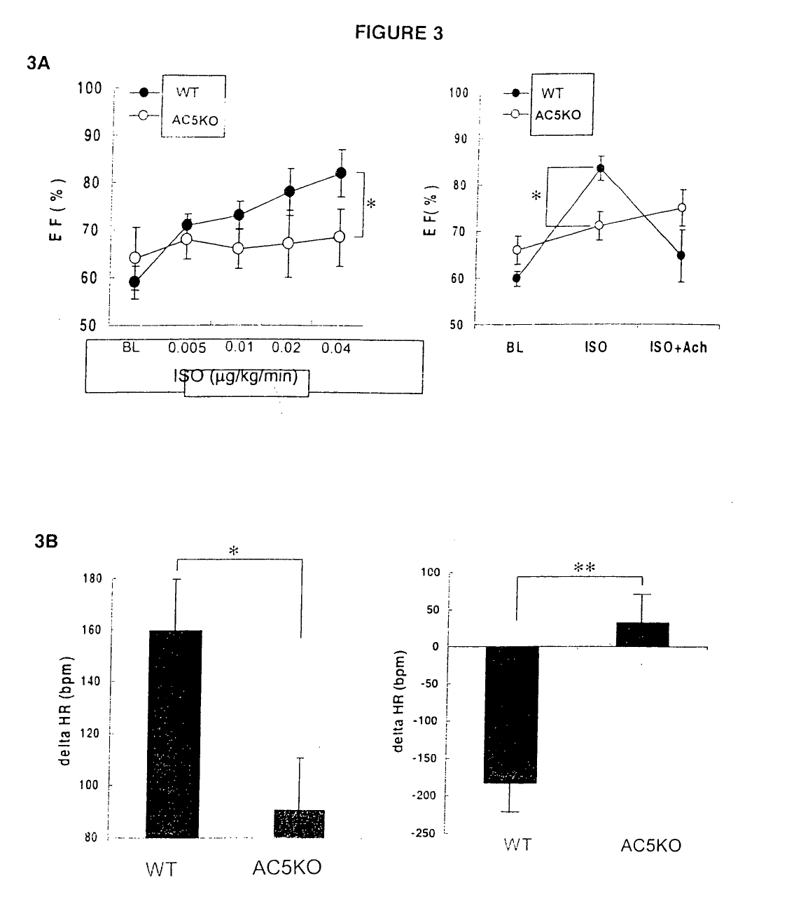

Inventors originally hypothesized that cardiac function, both basal and ISO-stimulated, would be depressed. The cardiac responses to i.v. ISO on LVEF and fractional shortening (FS) in AC5KO were attenuated as expected (FIGS. 3A and 3B). However, baseline cardiac function tended to be increased; LVEF (WT vs. AC5KO; 59±2.4% vs. 64±4.3%) and FS (26±1.4% vs. 29±2.7%). Muscarinic inhibition of ISO stimulated cardiac function, as measured by LVEF, was prominent in WT, as expected, but was abolished in AC5KO (FIG. 3A).

Parasympathetic (Muscarinic) Control of HR.

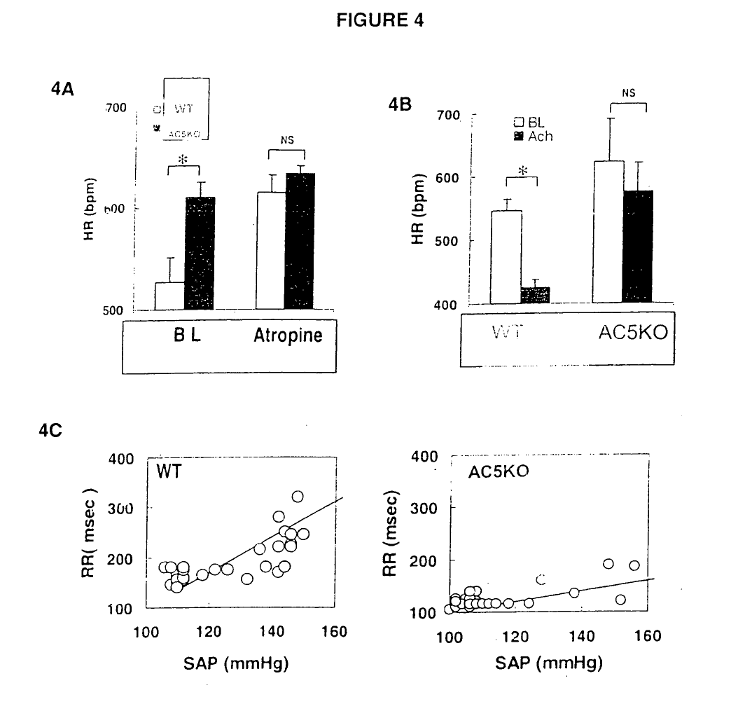

In the presence of ISO, Ach reduced HR in WT, but not in AC5KO (FIG. 3B). Baseline HR was significantly elevated in conscious AC5KO (FIG. 4A). Muscarinic stimulation in conscious WT with Ach (0.01 μg/g i.v.) decreased HR by 22% but significantly less (7.5%) in AC5KO (FIG. 4B). Phenylephrine (0.2 μg/g i.v.) increased systolic arterial pressure significantly in both WT and AC5KO, but induced less baroreflex mediated slowing of HR in AC5KO than in WT (FIG. 4C). The increase in HR following atropine (1 μg/g i.v.), in WT (102±22.2 beats/min) was not observed in AC5KO (19±7.5 beats/min) (FIG. 4A).

AC is critical to regulating cardiac contractility and rate, particularly in response to sympathetic activation. The rate of cardiac contraction is also under sympathetic control, but parasympathetic mechanisms may be even more important in its regulation, particularly with regard to reflex cardiac slowing. Importantly, AC is involved in parasympathetic modulation of cardiac function and HR, particularly in the presence of sympathetic stimulation.

A key mechanistic approach to understanding the role of AC in vivo is to alter AC genetically in the heart. Previous studies have overexpressed types 5, 6 and 8 AC in the heart. These studies found the expected increases in response to β-AR stimulation, but failed to observe any changes in parasympathetic control. Although targeted disruption of cardiac AC would be the preferred experimental approach to understand the mechanistic role of AC in the heart, this has not been accomplished previously. Some of the reasons are simply technical difficulties in producing the knockout. More importantly, there is not one AC, but rather 9 mammalian membrane-bound AC isoforms and significant heterogeneity exists in their distribution and biochemical properties, such that function of the isoforms may differ even within the same tissue. One laboratory deleted types 1, 3 and 8 AC, but the effects on cardiac function were not delineated. Inventors selected type 5 AC for deletion in this investigation, since this is the major AC isoform in the adult heart, which was confirmed in cardiac membrane preparations from AC5KO, where 30-40% of AC activity was lost. In addition, its biochemical properties reflect the overall signature of cardiac AC, in that types 5 and 6 are sensitive to direct inhibition by Gi.

It was predictable that increases in cardiac function and rate in responses to ISO would be diminished in AC5KO, as was demonstrated in this study. Similarly, inventors had expected that baseline cardiac function and HR would be reduced in AC5KO. This was not observed. In fact, baseline LVEF tended to be increased, and HR was significantly elevated in conscious AC5KO. Autonomically-mediated increases in HR can be attributed to increased sympathetic tone or reduced parasympathetic tone. Since the elevated HR was not likely due to enhanced sympathetic tone, i.e., inventors had already demonstrated that sympathetic responses were attenuated in AC5KO, inventors hypothesized that it was due to loss of parasympathetic inhibition. Inventors addressed this hypothesis with several experiments. First, it was demonstrated that muscarinic inhibition was reduced in AC5KO in the presence of enhanced β-AR stimulation with ISO. Neither the reduction in EF nor the decrease in HR induced by Ach in the presence of ISO in AC5KO were observed, whereas both effects were pronounced in the WT. Inventors next determined the effects of muscarinic stimulation in the absence of enhanced sympathetic stimulation. Inventors reasoned that these experiments would be best conducted in the conscious state. As already noted, in conscious AC5KO mice, HR is significantly elevated at baseline, which should facilitate an experiment with the object of demonstrating a decrease in HR. Muscarinic stimulation with Ach elicited the expected prominent decline in HR in WT, but a significantly blunted bradycardia in AC5KO. Conversely, atropine increased HR in WT, but not in AC5KO.

The above experiments indicate that muscarinic inhibition of HR in AC5KO is impaired, but do not demonstrate that this mechanism plays a role, in vivo, under conditions of enhanced parasympathetic tone. Perhaps the most intense parasympathetic tone can be elicited by activating reflex mechanisms. The best characterized reflex is the arterial baroreflex, which responds to an elevation in arterial pressure with bradycardia, mediated predominantly by parasympathetic mechanisms in the conscious animal. In WT, arterial pressure elevation with phenylephrine elicited intense bradycardia, which was blunted significantly in the AC5KO, indicating that parasympathetic mediation of reflex HR is impaired in AC5KO. These data taken together, provide convincing evidence in vivo that type 5 AC exerts a major role in parasympathetic regulation of cardiac function, in addition to its key role in sympathetic regulation, which has been recognized for some time. Thus, cAMP-mediated parasympathetic modulation of ventricular function and atrial function, i.e., HR, must be considered along with the more widely recognized mechanisms involving muscarinic modulation of potassium channel activity.

In summary, the AC5KO mouse provides an excellent model to study AC isoform specific regulation of the heart. The in vitro experiments confirmed that type 5 AC is the major isoform in the heart, and that in vivo, ISO stimulation of cardiac function and rate were blunted. Since type 5 AC is the major AC isoform expressed in the adult mouse heart, it was surprising to find no effect on baseline cardiac function, but rather an increase in HR, despite reduced baseline AC activity. Paradoxically, the increased basal HR, is more likely related to a loss of parasympathetic restraint, since loss of sympathetic stimulation would act in the opposite direction. The blunted parasympathetic restraint was also observed in response to baroreflex mediated bradycardia, and conversely, atropine induced less tachycardia in the AC5KO than in the WT. Thus, type 5 AC regulates cardiac inotropy and chronotropy through both the sympathetic and parasympathetic arms of the autonomic nervous system.

### EXAMPLE 3

AC Assay

Hearts were dissected from the mice, and membrane preparations were prepared as described previously. Protein concentration was measured by the Bradford method using bovine serum albumin as a standard. AC activity was measured as described previously. AC activity was linear within the incubation time up to 30 min. For the study of Ca 2+ inhibition, the membranes were treated first with EGTA to extract the endogenous Ca 2+ prior to the assay. Free Ca 2+ concentrations were obtained with the use of 200 μmol/L EGTA buffers as described previously. The experiments with Ca 2+ inhibition were conducted in the presence of 100 μmol/L isoproterenol (ISO)+100 μmol/L GTP.

Physiological Studies

Electrocardiogram (ECG) wires, a jugular vein catheter for drug infusion, and a femoral artery catheter for arterial pressure monitoring were implanted under anesthesia as described previously. Measurements of left ventricular ejection fraction (LVEF) were taken using echocardiography under anesthesia with 2.5% tribromomethanol (0.010-0.015 ml/g body wt) injected intraperitoneally (i.p.). Intravenous (i.v.) infusion of ISO (0.04 μg/kg/min i.v. for 5 min) was performed using an infusion pump. To examine the responses to a muscarinic agonist, acetylcholine (ACh) (25 mg/kg i.p.) was co-administered i.p. during the i.v. infusion of ISO (0.04 μg/kg/min). In addition, in conscious mice ACh (0.01 and 0.05 mg/kg), atropine (0.25, 1 and 2 mg/kg), or verapamil (0.75 mg/kg) were administered i.v., and the ECG was recorded. A recovery period of 15 min was allowed for the HR to return to baseline before administering the next drug. To examine HR responses to baroreflex hypertension, phenylephrine (0.2 mg/kg i.v.) was infused, and the ECG and arterial pressure were measured

Pathology

The pathological examination included assessment of body weight, heart weight, and light microscopy of hematoxylin- and eosin-stained sections of the left ventricle.

Radioligand Binding Assays and Western Blotting

Radioligand binding assays for α-AR were conducted using the above membrane preparations and 125 I-cyanopindolol as previously described 30. Western blotting for Gsα, Giα, Gqα, Gβα, α1-adrenergic receptor (α1-AR), α-adrenergic receptor kinase (α-ARK) and muscarinic receptor type 2 were conducted using either the membrane preparation or whole tissue homogenates.

Electrophysiological Studies

Whole-cell currents were recorded using patch-clamp techniques. Cell capacitance was measured using voltage ramps of 0.8 V/s from a holding potential of −50 mV. All experiments were performed at room temperature. Ca²⁺ channel currents (ICa) were measured with an external solution (mmol/L): CaCl₂ or BaCl₂ 2; MgCl₂ 1; tetraethyl ammonium chloride 135; 4-aminopyridine 5; glucose 10; and HEPES, 10 (pH 7.3). The pipette solution contained (mmol/L): Cs-aspartate, 100; CsCl, 20; MgCl₂, 1; MgATP, 2; GTP, 0.5; EGTA, 5 or 1,2-bis (2-aminophenoxy)ethane-N,N,N′,N′-tetraacetic acid (BAPTA), 10 and HEPES, 5 (Ph 7.3). For potassium (K⁺) channel current recordings, the external solution was normal Tyrode's solution (mmol/L): NaCl, 135; CaCl₂, 1.8; MgCl₂, 1; KCl, 5.4; glucose, 10; HEPES, 10 (pH 7.3). Nifedipine (10 μmol/L) was added to block L-type Ca²⁺ channel currents. The patch pipette solution contained (mmol/L): potassium aspartate, 110; KCl, 20; MgCl₂, 2; ATP, 2; GTP, 0.5; EGTA, 5; HEPES, 5 (pH 7.3).

Statistical Analysis

All data are reported as mean±SEM. Comparisons between AC5−/− and WT values were made using a Student's t-test. For statistical analysis of data from multiple groups, one way ANOVA was used, with Bonferroni post hoc test. P<0.05 was taken as a minimal level of significance.

Results

AC Activity Was Decreased in the Heart of AC5KO In Vitro

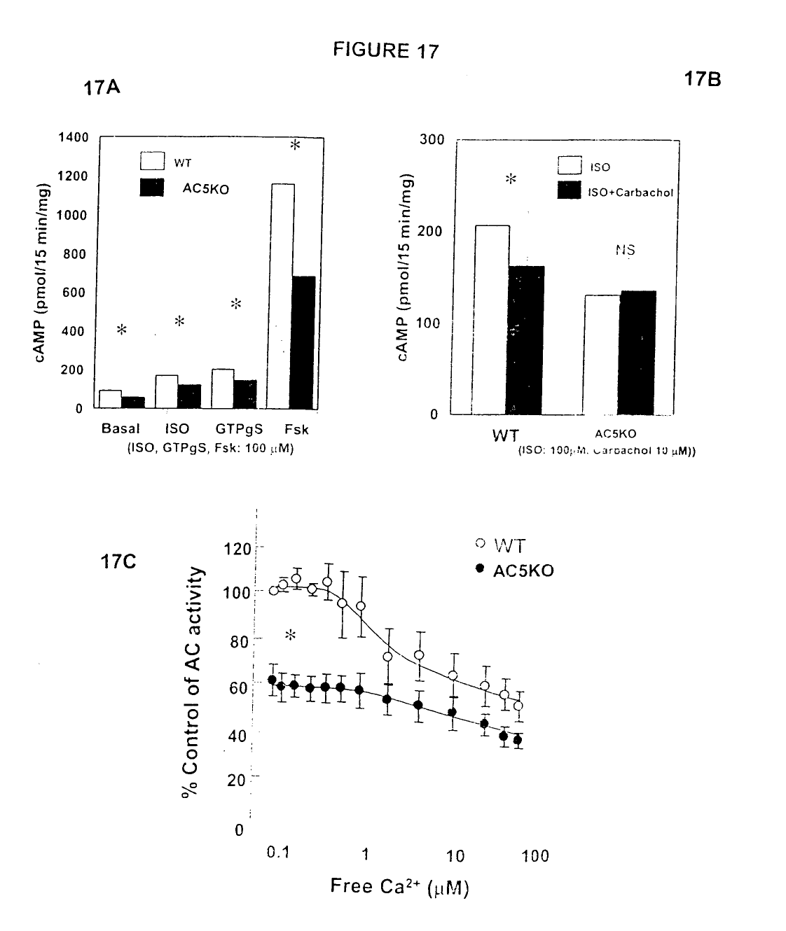

cAMP production in membranes from the hearts of AC5KO and WT mice at the age of 6 months were examined. The steady state AC activity in the membrane preparation was determined as the maximal capacity of cAMP production in the presence of ISO (100 μmol/L ISO+100 μmol/L GTP), guanosine-5′-O-(3-triophosphate) GTPγS (100 μmol/L), or forskolin (100 μmol/L) (FIG. 17A). AC activity was decreased in AC5KO relative to that of WT by 35±4% (basal), 27±5% (ISO), 27±2% (GTPγS), and 40±5% (forskolin). More specifically, ISO increased AC activity by 78±6 pmol/15 min/mg in WT, but only 64±4 pmol/15 min/mg in AC5KO, indicating that the response to ISO was attenuated in AC5KO. These data indicate that type 5AC is responsible for approximately 30%-40% of the total AC activity in the mouse heart Carbachol (10 μmol/L), a muscarinic agonist, decreased ISO stimulated activity by 21±3% in WT, but this was hardly detectable in AC5KO (FIG. 17B), indicating that muscarinic (Gi induced inhibition of the AC activity is markedly attenuated in AC5KO.

Regulation of AC Activity by Free Ca 2+

To investigate the modulation of AC activity by free Ca 2+, cAMP production was examined in membranes from the hearts of WT and AC5KO at different Ca²⁺ concentrations in the presence of ISO (100 μmol/L ISO+100 μmol/L GTP) (FIG. 3C). The ISO-stimulated AC activity was inhibited by increasing concentrations of Ca²⁺ as expected in WT. The Ca²⁺ inhibition of AC activity was impaired in AC5KO . The reduction in magnitude of inhibition was most apparent in AC5KO, i.e., in the submicromolar range of Ca²⁺ (FIG. 3C).

Basal Cardiac Function Was Not Decreased, But the Response to ISO and Muscarinic Inhibition of ISO Were Impaired

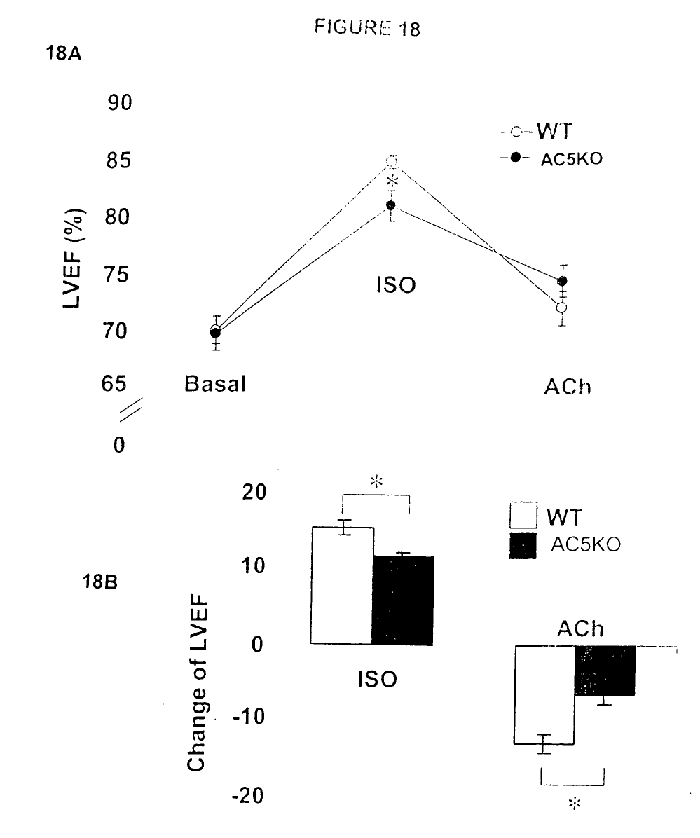

The cardiac responses to i.v. ISO on LVEF in AC5KO were attenuated as expected (FIG. 18). However, baseline cardiac function was not different between WT and AC5KO (FIG. 1 8A); LVEF (WT vs. AC5KO: 70±1.2% vs. 70±1.5%, n=10-11); fractional shortening (WT vs. AC5KO : 33±0.9% vs. 33±1.0%, n=10-11) (see Table 1). Muscarinic inhibition of ISO stimulated cardiac function, as measured by LVEF, was prominent in WT, as expected, but was attenuated in AC5KO (Figure FIG. 18B), suggesting that muscarinic inhibition of α-adrenergic stimulation was impaired.

TABLE 1

| | WT(n) | AC5KO |
|---|---|---|
| Age(month) | 4.4 ± 0.1(15) | 4.2 ± 0.2(15) |
| BW(g) | 25 ± 1.0(15) | 27 ± 1.0(14) |
| LV/BW(mg/g) | 3.9 ± 0.1(9) | 4.1 ± 0.2(8) |
| HR(bpm) | 523 ± 11(15) | 613 ± 8(14)* |
| LVDD(mm) | 3.9 ± 0.1(11) | 4.0 ± 0.1(10) |
| LVSD(mm) | 2.6 ± 0.09(11) | 2.7 ± 0.1(10) |
| LVEF(%) | 70 ± 1.2(11) | 70 ± 1.5(10) |
| % FS | 33 ± 0.9(11) | 33 ± 1.0(10) |

Data are mean ± SEM
HR is under conscious state and other functional data are under anesthesia
LVEF: Left Ventricular Ejection Fraction
LVDD: LV end-diastolic diameter
LVSD: LV end-systolic diameter
% FS: % fractional shortening
*P < 0.01

Parasympathetic (Muscarinic) Control of HR

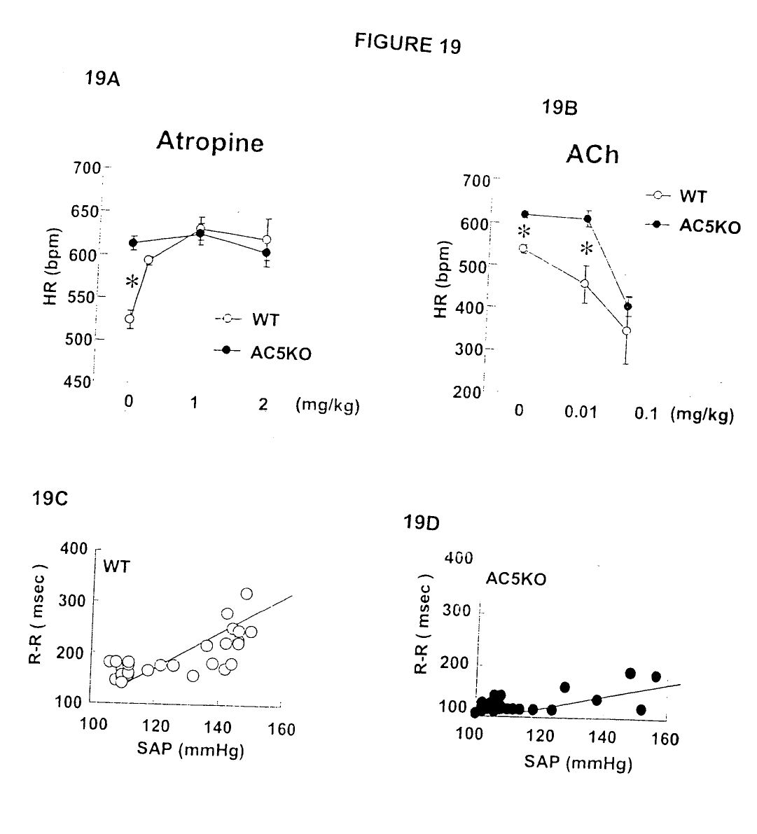

Baseline HR was significantly elevated in conscious AC5KO (WT vs. AC5KO: 523±11 vs. 613±8 beats/min, P<0.01, n=14-15) (Table 1). The increase in HR following muscarinic receptor blockade by atropine (1 mg/kg i.v.) in WT was not observed in AC5KO (FIG. 19A). Muscarinic stimulation in conscious WT with ACh (0.01 mg/kg i.v.) decreased HR by 15% but significantly less (1.3%) in AC5KO (FIG. 19B). However, high doses of ACh (0.05 mg/kg i.v.) decreased HR similarly in both WT and AC5KO. At the higher doses of ACh, it is possible that the lack of AC5 inhibition was overwhelmed. In contrast, verapamil, which decreases HR through a non-muscarinic mechanism, reduced HR in AC5KO and WT similarly (−33±10 vs. −36±10 beats/min). These findings suggest that muscarinic inhibition was impaired in the conscious state in the absence of ISO-stimulation in AC5KO. To confirm that muscarinic, and therefore parasympathetic, neural regulation of the heart was changed, phenylephrine (0.2 mg/kg i.v.) was injected to elevate arterial pressure transiently through vasoconstriction and to induce baroreflex-mediated slowing of HR. Phenylephrine increased systolic arterial pressure similarly in both WT and AC5KO. However, the degree of HR slowing was significantly less in AC5KO than in WT (FIG. 19C), suggesting that the baroreflex, most likely through its parasympathetic control, was attenuated in AC5KO.

β-AR Binding Assay and Western Blotting

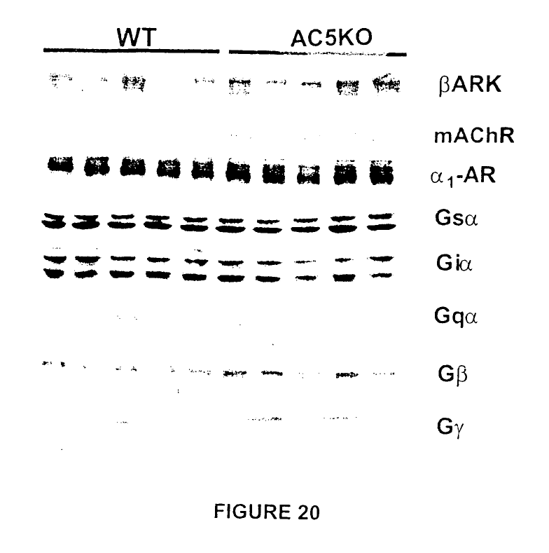

Because a decrease in the content of AC may be compensated by changes in the expression of the other molecules involved in cAMP signaling, the protein expression of other related molecules, such as α-AR was examined by radioligand binding assays, and Gsα, Giα, Gqα, Gβα as well as α-ARK, α1-AR, and muscarinic receptor type 2 (the major isoform in the adult heart) by western blotting. The expression of α-AR was not different (Kd: WT 102±17 pmol/L, AC5−/−115±29 pmol/L; Bmax: WT 36±5 fmol/mg, AC5−/−31±4 fmol/mg; n=5, P=NS), nor was the expression of Gsα, Giα, Gqα, Gβα, α-ARK, α1-AR, and muscarinic receptor type 2 (FIG. 20).

K⁺ Current Activity

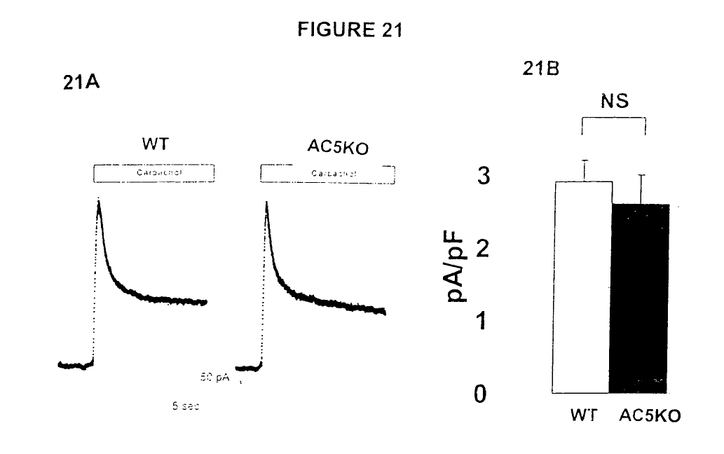

Normal pacemaker activity is also regulated by vagal stimulation via muscarinic receptor-coupled K⁺ channels, i.e., GIRK (G-protein-activated inwardly rectifying K⁺ channel), independent of intermediary signaling 32,35-38 . To determine whether enhanced baseline HR and blunted response to muscarinic agonists in AC5KO are due to changes in the K⁺ channel, muscarinic receptor coupled K⁺ channel currents were examined in atrial myocytes. FIG. 21 A shows representative atrial K⁺ channel currents induced by carbachol (10 μmol/L) recorded in WT and AC5KO myocytes. Rapid application of carbachol elicited an outward K⁺ current via Gi proteins. The carbachol-induced currents rose quickly to a peak and then decayed slowly to a steady level. The peak amplitude and decay time were similar between WT and AC5KO myocytes (FIG. 21B). These results indicate that coupling between muscarinic receptors and the Gi-gated K⁺ channel are not altered in AC5KO myocytes.

Basal Ca 2+ Channel Activity and Response to ISO

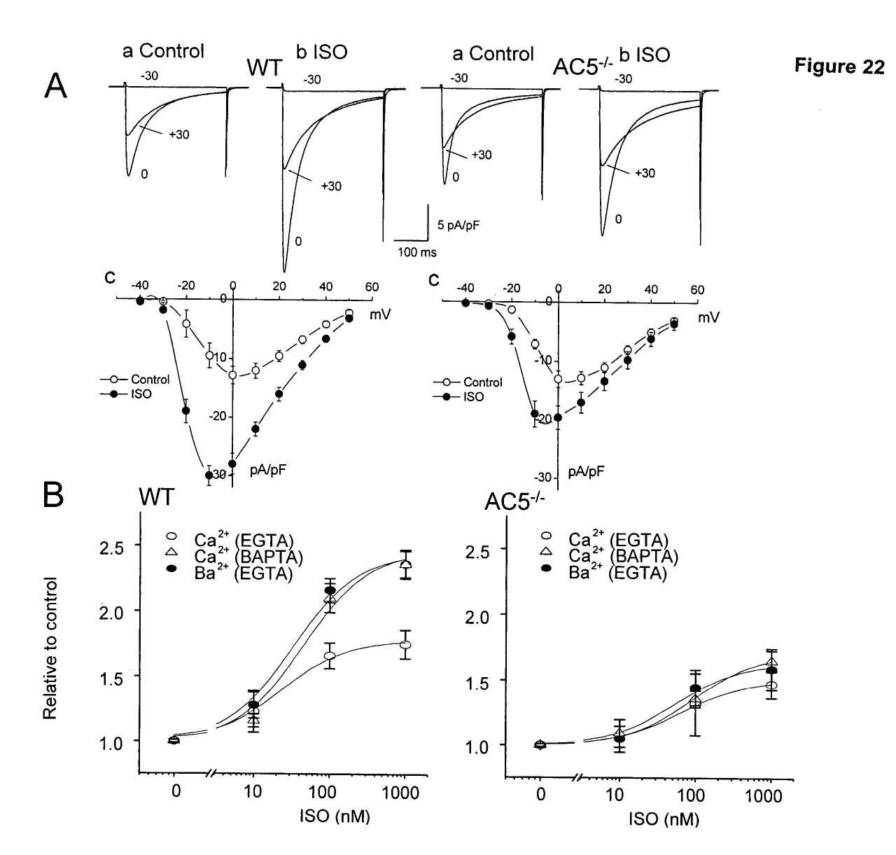

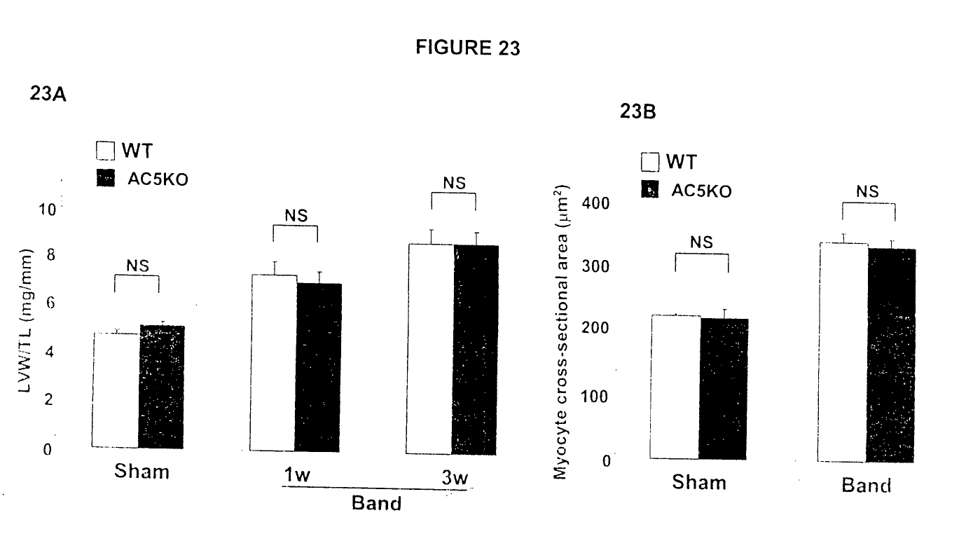

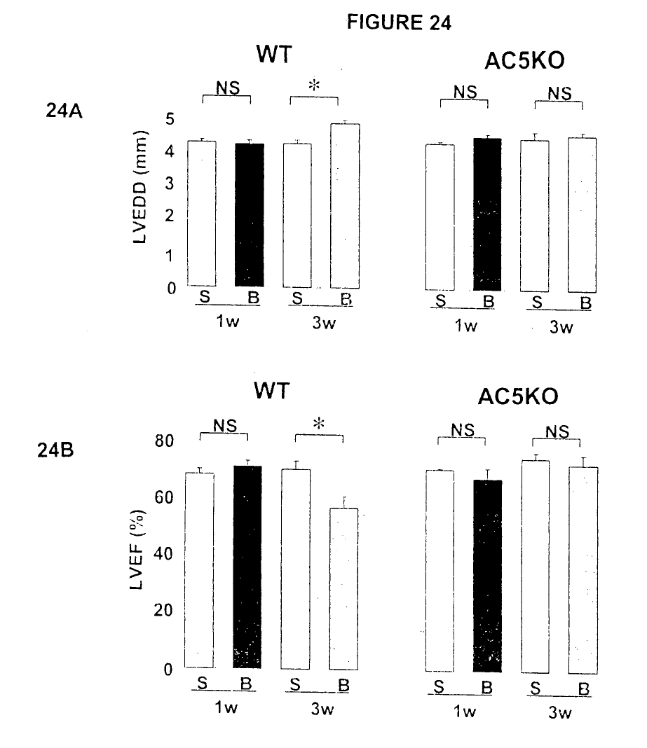

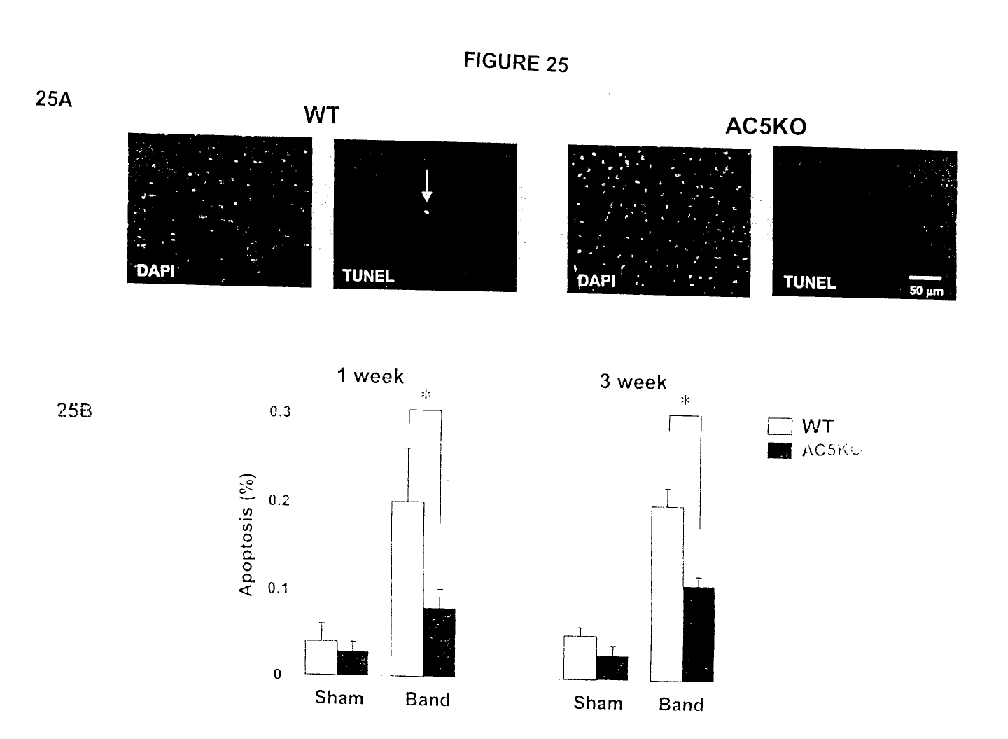

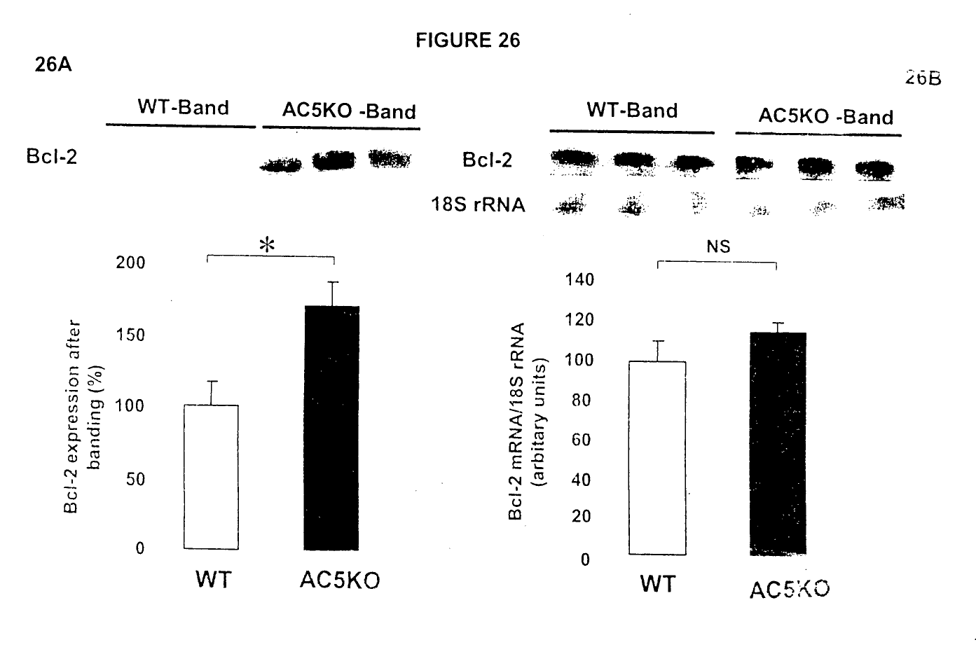

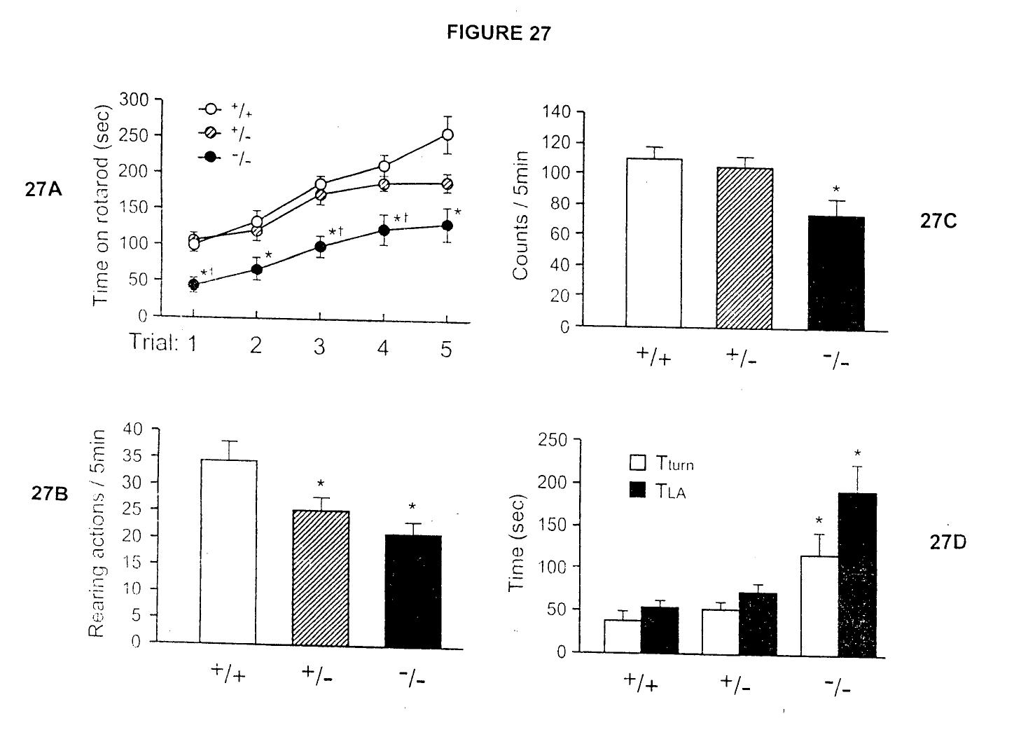

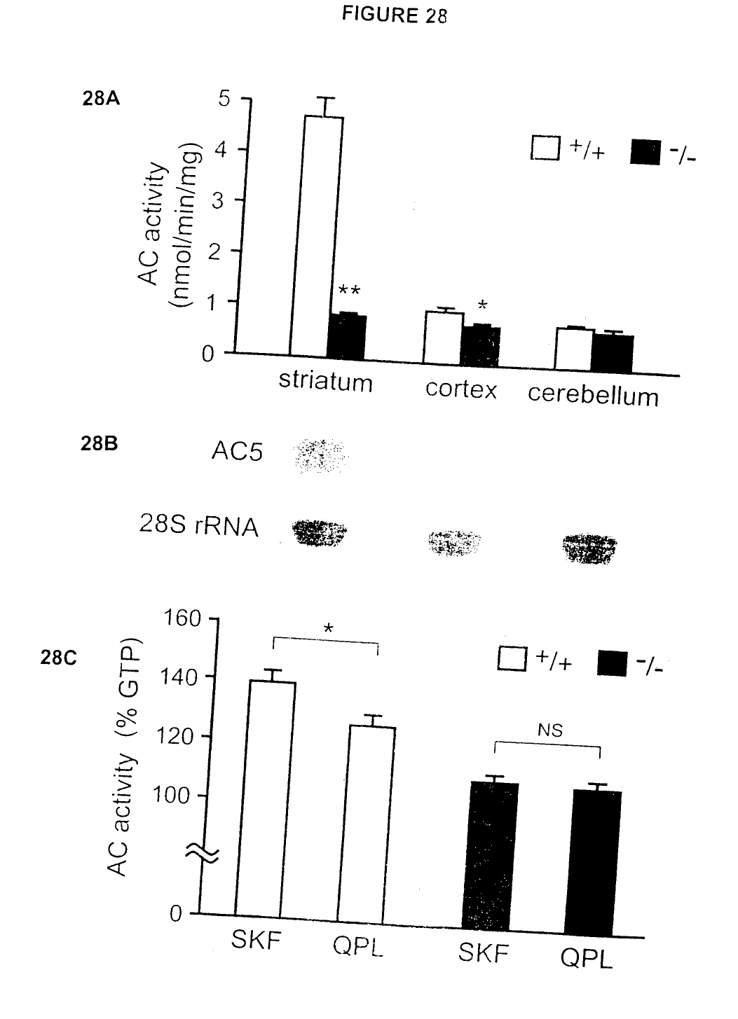

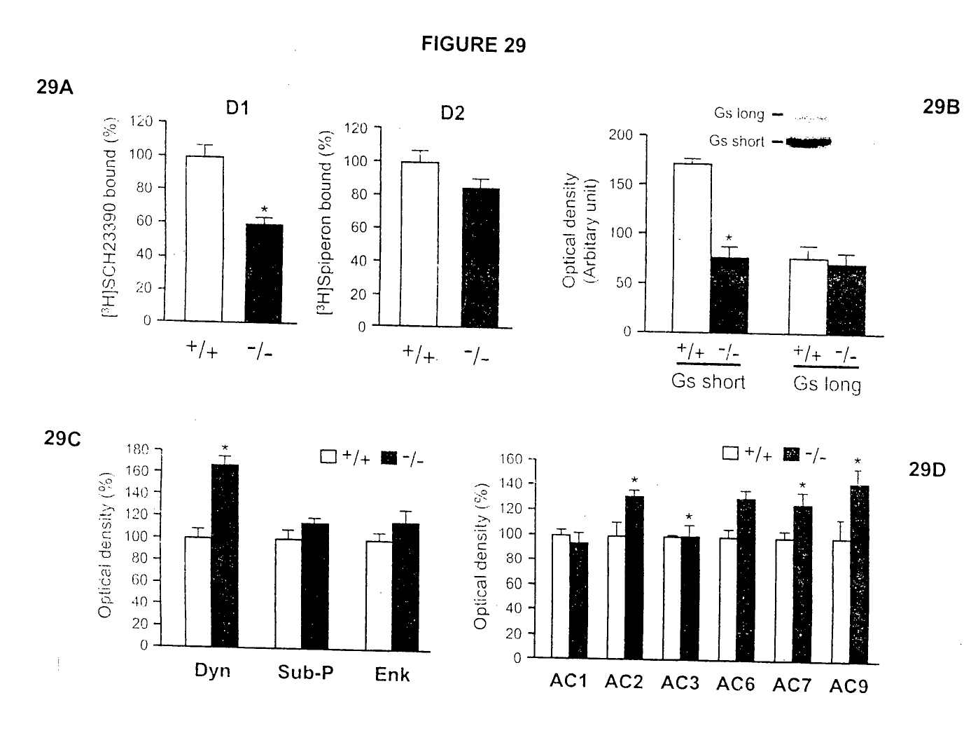

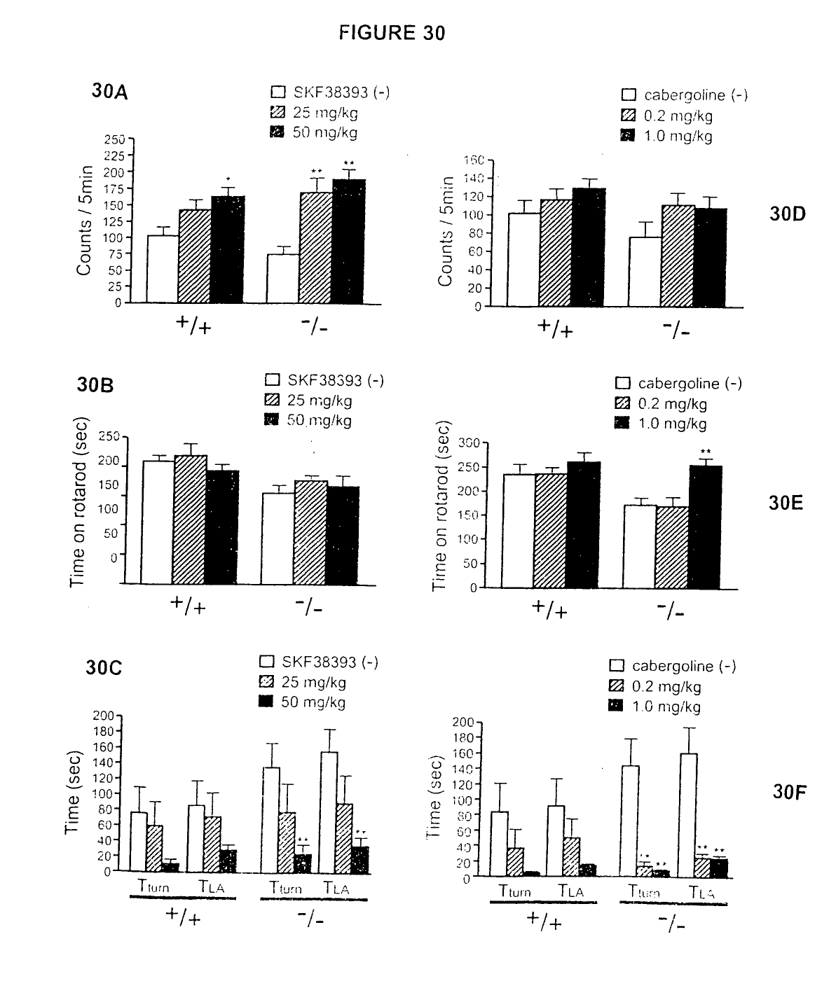

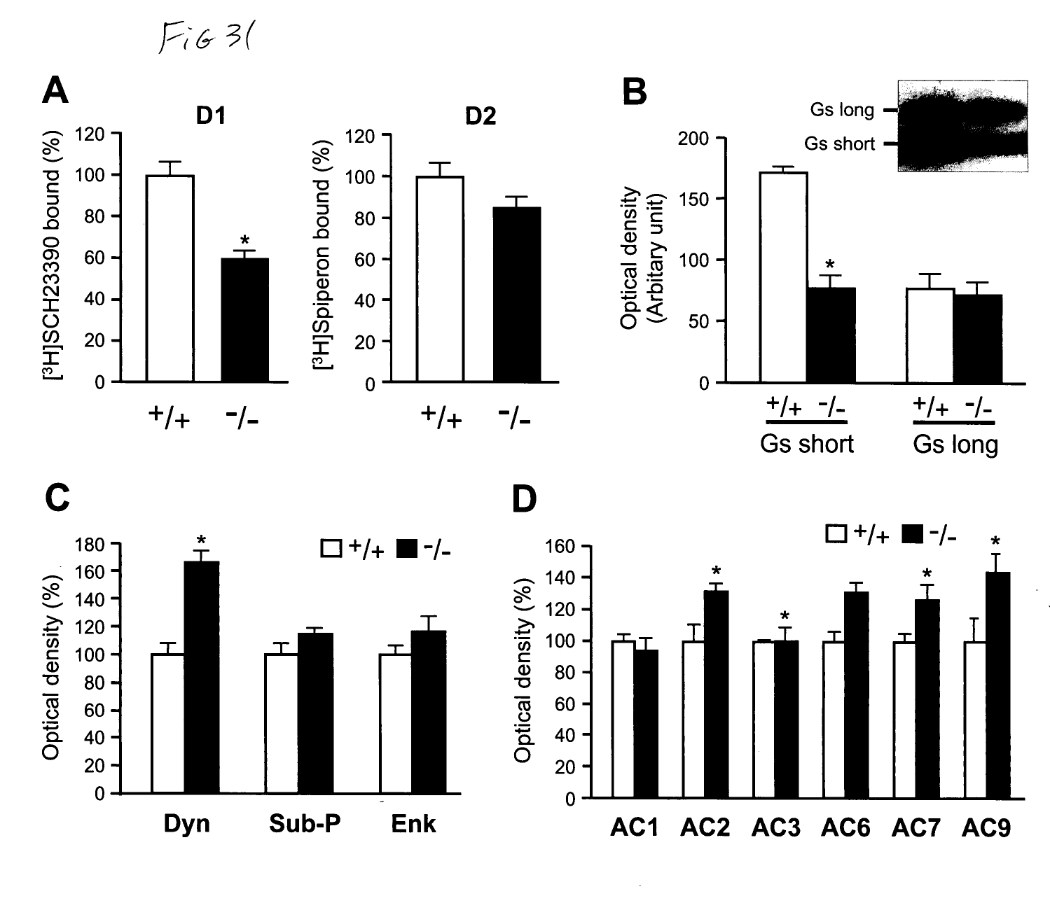

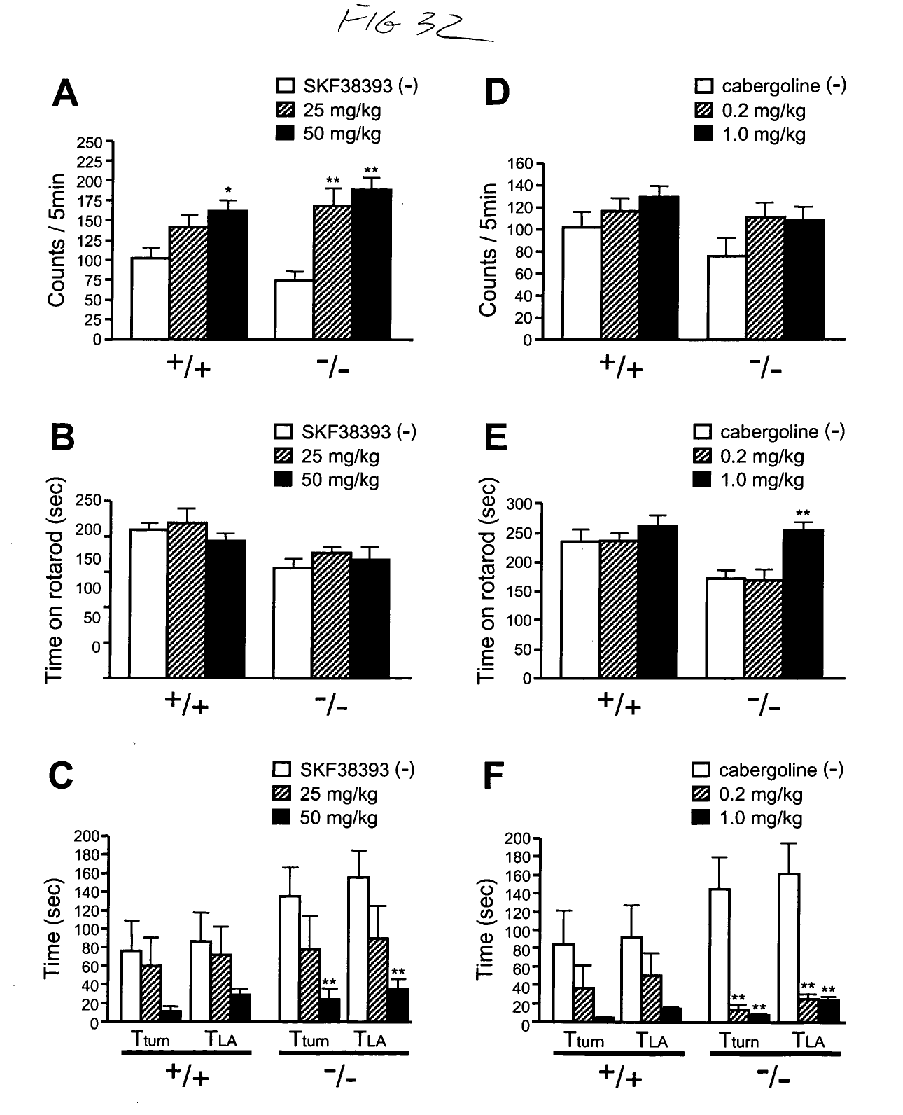

Ca²⁺ influx through L-type Ca²⁺ channels is essential for cardiac contractility, thus basic Ca²⁺ channel properties were characterized. Peak inward ICa amplitude (with 5 mmol/L EGTA in the pipette solution), normalized relative to cell capacitance (ICa density), was similar in myocytes isolated from AC5KO (7.1±0.3 pA/pF, n=69) and WT (6.7±0.3 pA/pF, n=55). T½ (half decay time of ICa at +10 mV was 21.9±1.4 msec and 21.0±1.4 msec, for AC5KO and WT, respectively. These data suggest that changes in AC activity did not directly influence Ca²⁺ channel density or inactivation kinetics. In previous studies, it was proposed that AC activity and subsequent cAMP synthesis, which modulate Ca²⁺ channel activity, are regulated by Ca²⁺ entering through the Ca²⁺ channel. The effects of ISO on ICa using procedures designed to modulate the cytoplasmic Ca²⁺ concentration with two different Ca²⁺ chelators, EGTA, which has slower Ca²⁺ binding kinetics, and BAPTA, which has faster Ca²⁺ binding kinetics, and with the use of extracellular barium (Ba²⁺), which permeates the Ca²⁺ channel but does not trigger Ca²⁺ of the sarcoplasmic reticulum (SR), were compared. FIG. 22A shows a typical example of the effect of ISO (1 μmol/L) on ICa in WT and AC5KO. In these experiments myocytes were dialyzed with BAPTA, and peak ICa amplitude, as the function of voltage (I-V relationships) before and after exposure to ISO, was determined. In both groups, ISO increased the current amplitude at all test potentials and also shifted the I-V relationships toward more negative potentials. However, the maximal increase was significantly less in AC5KO. Analysis of cumulative dose-response effects of ISO (FIG. 22B) revealed that, when either BAPTA or Ba²⁺ was used, the maximum response of the Ca²⁺ channel to ISO was significantly augmented (˜2.4-fold) compared to cells dialyzed with EGTA (˜1.7-fold), suggesting that Ca²⁺ inhibited Ca AC5KO channel activity in WT 20 . In contrast, the responses of AC5KO myocytes to ISO were essentially the same in all three conditions (˜1.5-fold), suggesting that Ca²⁺ -mediated inhibition of Ca²⁺ channel activity was markedly diminished in AC5KO. These results suggest that intracellular Ca²⁺ can inhibit β-AR-mediated activation of Ca²⁺ channels, presumably through directly inhibiting cardiac AC activity 20, and that type 5 AC is a major target of this inhibition (FIG. 22B).

Discussion

Using a mouse model with disruption of the major AC isoform (AC5KO), it was predictable that increases in cardiac function in response to ISO would be diminished in AC5KO, as was demonstrated in this study. Indeed the decrease in cardiac responsiveness to ISO in vivo paralleled the data in vitro on AC activity. Since overexpression of type 5 AC in the heart enhanced cardiac function, it had been expected that baseline cardiac function and HR would be reduced in AC5KO, which was not observed. Despite the decrease in AC activity, basal cardiac function and HR were not decreased in AC5KO. Actually, HR was significantly elevated in conscious AC5KO. Although not completely understood yet, it is proposed that at least three mechanisms, those which are impaired in AC5KO: 1) muscarinic inhibition of AC activity, 2) baroreflex restraint of HR, and 3) Ca²⁺-mediated inhibition of AC activity. Since the elevated HR was not likely due to enhanced sympathetic tone, i.e., sympathetic responses were attenuated in AC5KO in both in vivo and in vitro experiments, it was hypothesized to be due, at least in part, to the loss of parasympathetic inhibition, since type 5 AC is a major Gi-inhibitable isoform in the adult heart 17, 18. To confirm this, it was demonstrated that muscarinic stimulation, which inhibits cardiac function and HR, was attenuated in AC5KO both in the presence and absence of enhanced β-AR stimulation with ISO. Conversely, atropine increased HR in WT, but not in AC5KO, supporting the concept that the higher baseline HR was due to the loss of parasympathetic restraint. Furthermore, the arterial baroreflex slowing of HR, which occurs through parasympathetic nerves, was also blunted in the AC5KO. Therefore, at any given arterial pressure there is less baroreflex restraint, resulting in elevated HR. These data provide convincing evidence in vivo that type 5 AC exerts a major role in parasympathetic regulation of cardiac function in addition to its key role in sympathetic regulation, which has been recognized for some time. Thus, AC-mediated parasympathetic modulation of ventricular function and atrial function, i.e., HR, must be considered along with the more widely recognized mechanisms involving muscarinic modulation of K⁺ channel activity and muscarinic regulation at the level of membrane receptors, or Gi. To support this conclusion, the K⁺ current in atrial myocytes and the expression of G proteins, β-ARK, muscarinic receptor type 2, and β- and α 1-AR were not altered in AC5KO. Finally, it is also conceivable that the impaired Ca²⁺ inhibition of AC also contributes to the increased HR at baseline. In order to conclude that tachycardia in AC5KO was due to the loss of parasympathetic restraint, it is important to rule out the possibility that some other compensatory pathway did not cause the tachycardia. This possibility is unlikely for several reasons. First, the increase in HR is not compensatory, but is actually opposite to the prediction that reduced contractility and HR would be expected from disruption of AC. Although unlikely, it is still possible that the resetting autonomic activity in the brain, or some mechanism at the level of Ca²⁺ channels, could be involved. Type 5 AC is also located in the striatum of the brain, and disrupting this isoform of AC does alter dopaminergic transmission in the brain. However, it is more likely that parasympathetic stimulation leads to activation of muscarinic receptors and Gi to inhibit type 5 AC in the heart, which results in restraint on baseline HR. In the absence of type 5 AC, this restraint is lost and HR rises, as was observed in the AC5KO mice in this investigation. It is important to note that the bradycardia resulting from pharmacologic muscarinic inhibition with ACh was attenuated in AC5KO, indicating that the mechanism is localized to the heart and does not reside in the CNS. In further support of this conclusion are the complementary in vitro data from cardiac membranes. HR is thought to be regulated at the level of the muscarinic receptor, or Gi, or GIRK 38 . In the current investigation, coupling between muscarinic receptors and the GIRK was not altered in AC5KO. In view of the major alteration in muscarinic control in AC5KO. These results lead to the conclusion that cardiac rate of contractility is also regulated at the level of AC.

In cardiac muscle, Ca²⁺ influx through the L-type Ca²⁺ channel is the primary pathway for initiation and maintenance and for the modulation of contractility by catecholamines. The increase in ICa by the β-adrenergic agonist, ISO, occurs via a cascade of events leading to PKA-mediated phosphorylation of components associated with the Ca²⁺ channel. In turn, cardiac AC is regulated negatively by low concentrations of Ca²⁺. This mechanism was also impaired in AC5KO. The extent to which this mechanism is impaired in AC5KO must be interpreted cautiously, since small changes in experimental conditions can influence the magnitude of the results. Our finding suggested that under physiological conditions, an increase in Ca²⁺ entry and inhibition of type 5 AC, leading to decreased phosphorylation and thus activity of the Ca²⁺ channel, can work synergistically to provide an intrinsic feedback mechanism for cellular Ca²⁺ homeostasis. Thus, due to the lack of Ca²⁺-inhibitable type 5 AC in AC5KO, this negative feedback inhibition of the L-type Ca²⁺ channel may be lost. This loss may account for, at least in part, the maintained cardiac function in AC5KO. It is also important to consider the possibility that differences in SR loading and Ca²⁺ handling may have affected the response to ISO. However, in previous studies, it was found that mouse ventricular myocytes that AC activity and subsequent cAMP synthesis, which modulate Ca²⁺ channel activity, are regulated by the Ca²⁺ entering through the Ca²⁺ channel rather than by Ca²⁺ released from the SR stores in mouse ventricular myocytes. Another consideration is potential changes in calmodulin levels, which could regulate Ca²⁺-dependent Ca²⁺ channel inactivation. However, AC5KO mice did not exhibit changes in Ca²⁺ channel amplitude or inactivation time course. Furthermore, calmodulin content assessed by western blotting did not change in the AC5KO (data not shown). In summary, since type 5 AC is the major AC isoform expressed in the adult mouse heart, it was surprising to find no effect on baseline cardiac function, but rather an increase in HR, despite reduced baseline AC activity. Both the increased basal HR and blunted baroreflex-mediated bradycardia may be related to a loss of parasympathetic restraint and reduced Ca²⁺ regulation of AC. Thus, type 5 AC regulates cardiac inotropy and chronotropy through the parasympathetic arm of the autonomic nervous system, as well as through the sympathetic arm. Therefore, these new mechanisms for regulation of parasympathetic/sympathetic interactions and Ca²⁺-mediated regulation conveyed by this specific AC isoform in the heart will likely have broad significance for the understanding of the pathophysiology and treatment of heart failure as well as in normal cardiac regulation.

### EXAMPLE 4

Adenine or its Analogs Inhibit AC5

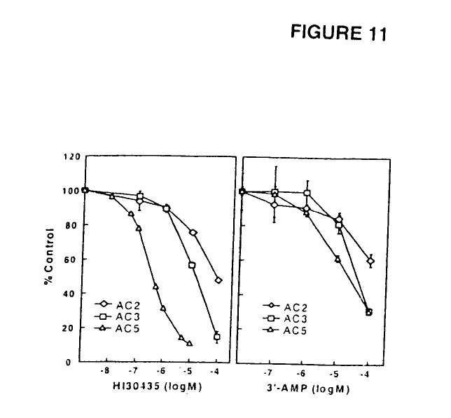

As described previously, “HI30435” showed a high selectivity to inhibit AC5. The result from a dose-response analysis and the determination of the IC50 values are discussed below and shown in FIG. 11.

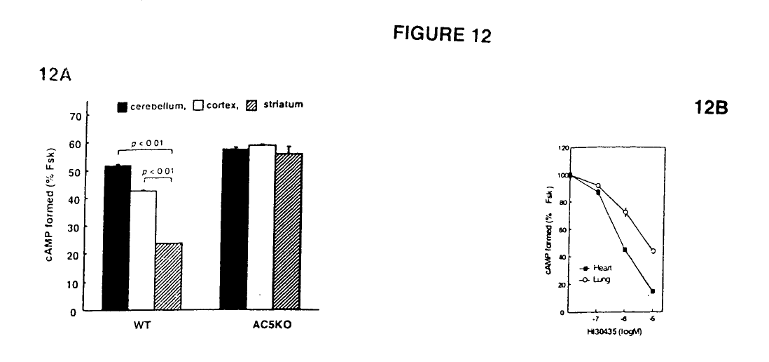

Inventors will first determine the selectivity among the AC isoforms (FIG. 12). The relative potency of HI30435, in comparison to classic AC inhibitor (3′-AMP) is shown as an example. HI30435 potently inhibited AC5 while that inhibited AC2 and AC3 only to a modest degree. The IC₅₀ values were calculated to be 0.32 micro M for AC5, 11.1 micro M for AC3, 65.3 micro M for AC2. The selectivity ratio of HI30435 was 207 between AC5 and AC2. 3′-AMP showed a weak selectivity for AC5 in inhibiting AC catalytic activity. The IC₅₀ values were calculated to be 14.6 micro M for AC5, 30.2 micro M for AC3, 263 micro M for AC2. The selectivity ratio was 18 between AC5 and AC2. These data suggest that HI30435 is extremely specific and strong inhibitor for AC5. Most importantly, HI30435, but not NKY80, inhibited cAMP accumulation in intact H9C2 cells. This suggests that membrane penetration of these compounds is important for biological activity and that HI30435, but not NKY80, has such a capability.

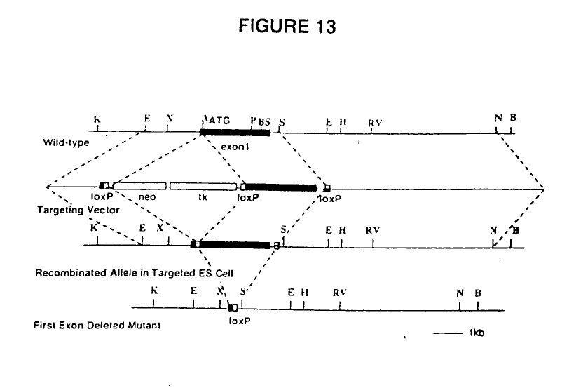

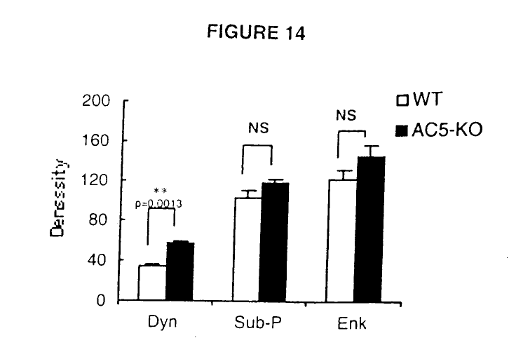

Inventors then determined the effect of compounds on AC activity in various brain regions (FIG. 13). As stated below, the brain expresses the neuronal subgroup of AC isoforms (AC1 and AC8) that is not included in our initial screening. As shown below, HI30435 inhibited striatal AC activity most potently, but only to a small degree in the cortex and the cerebellum (FIG. 13, left). These findings are in agreement with those using AC isoforms. Similarly, the effect of HI30435 was compared between the heart and lungs. As shown below, the inhibitory effect of HI30435 was greater in the heart relative to that of lungs. It should be noted, however, that the degree of organ-selectivity of this compound is smaller than that of AC isoform-selectivity. This is due to the presence of multiple AC isoforms in each organ. New compounds that will be examined in the future will follow the same process.

AC5 versus AC6: Since AC6 has a biochemical property very similar to that of AC5, and AC6 is also expressed in the striatum, greater degree of inhibition of striatal AC than that of cortical and cerebella AC may also represent the inhibition of AC6 in the striatum. To address this issue, inventors examined the effect of HI30435 on striatal, cortical and cerebellar AC in AC5KO (FIG. 13, left). The degree of inhibition in each brain region became much smaller and similar in AC5KO in comparison to that in WT. In particular, striatal inhibition was dramatically attenuated. Inhibition of cerebella AC was also attenuated, but only very slightly. These findings suggest that dramatic inhibition of striatal AC in WT results from the inhibition of AC5, not AC6. Similarly, inventors will examine the effect of HI30435 on cardiac AC in WT and AC5KO. The inhibition of cardiac AC in WT may represent the inhibition of both AC5 and AC6. However, the inhibition in AC5KO should represent that of only AC6. Thus, the comparison between WT and AC5KO would demonstrate the contribution of AC5 activity in each tissue subtype.

### EXAMPLE 5

Disruption of Type 5 AC Gene Preserves Cardiac Function Against Pressure Overload

Chronic pressure overload is a cause of heart failure. In response to pressure overload, the myocardium undergoes adaptive hypertrophy in order to maintain cardiac output against the increased afterload. Prolonged pressure overload eventually leads to heart failure as reflected by the dilatation of the Left Ventricle (LV) and a decrease in cardiac contractility, eg. Left Ventricular Ejection Fraction (LVEF). Pressure overload also results in apoptosis, which is thought to be part of the mechanism of cardiac decompensation. The role of the beta adrenergic (β-AR) signaling is well defined as a primary defense against acute stress or changes in hemodynamic load; however, uncertainty remains about its role in the pathogenesis of heart failure. The purpose of the experiment below was to examine the effects of chronic presssure overload induced by aortic banding in AC5KO and Wild Type (WT) controls. Specifically, the extent to which LV hypertrophy and apoptosis developed in response to pressure overload and and the resultant effects on cardiac function were examined.

Aortic Banding

Transverse aortic banding or sham operation was performed in 4-6 month-old homozygous AC5KO and WT littermates. The method of imposing pressure overload in mice has been described in other work. Mice were anesthetized intraperitoneally with a mixture of ketamine (0.065 mg/g), xylazine (0.013 mg/g), and acepromazine (0.002 mg/g). Mice were ventilated via intubation with a tidal volume of 0.2 ml and a respiratory rate of 110 breaths per minute. The left side of the chest was opened at the second intercostal space and the transverse thoracic aorta was constricted. To measure the pressure gradient across the constriction, two high-fidelity catheter tip transducers (1.4F; Millar Instruments Inc.) were used at one week after aortic banding. One was inserted into the right carotid artery and the other into the right femoral artery, and they were advanced carefully to the ascending aorta and abdominal aorta, respectively, where pressures were measured simultaneously.

Echocardiography

Mice were anesthetized as already discussed. Echocardiography was performed using ultrasonography (Sequoia C256; Acuson Corporation) (12-14). A dynamically focused 13 MHz annular array transducer was applied from below, using a warmed saline bag as a standoff. M-mode measurements of LV internal diameter were made from more than three beats and averaged. Measurements of the LV end-diastolic diameter (LVEDD) were taken at the time of the apparent maximal LV diastolic dimension, while measurements of the LV end-systolic diameter (LVESD) were taken at the time of the most anterior systolic excursion of the posterior wall. LVEF was calculated by the cubic method: LVEF (%)=[(LVEDD)³−(LVESD)³]/(LVEDD)³.

Evaluation of Apoptosis

DNA fragmentation was detected in situ by using TUNEL staining (14). Briefly, deparaffinized sections were incubated with proteinase K and DNA fragments labeled with biotin-conjugated dUTP and terminal deoxyribonucleotide transferase and visualized with FITC-ExtrAvidin (Sigma-Aldrich). Nuclear density was determined by manual counting of 4′,6-diamidine-2′-phenylindole dihydrochloride (DAPI)-stained nuclei in six fields of each animal using the x40 objective, and the number of TUNEL-positive nuclei was counted by examining the entire section using the same power objective. Limiting the counting of total nuclei and TUNEL-positive nuclei to areas with a true cross section of myocytes made it possible to selectively count only those nuclei that were clearly within myocytes. For some samples, triple staining with propidium iodide (Vector Laboratories Inc.), TUNEL, and anti-α-sarcomeric actin antibody (Sigma-Aldrich), and subsequent analyses using confocal microscopy, were performed in order to verify the results obtained with light microscopy.

Myocyte Cross-Sectional Area

Myocyte cross-sectional area was measured from images captured from silver-stained 1-μm-thick methacrylate sections (14). Suitable cross sections were defined as having nearly circular capillary profiles and circular-to-oval myocyte sections. No correction for oblique sectioning was made. The outline of 100-200 myocytes was traced in each section. MetaMorph image system software (Universal Imaging Corp.) was used to determine myocyte cross-sectional area.

Western Blotting

Crude cardiac membrane fractions were prepared and separated on 4-20% SDS-polyacrylamide gel and blotted onto nitrocellulose membrane. Western blotting was conducted with anti-Bcl-2 and anti-Bax antibodies (BD Biosciences). Expression of these proteins was quantified by densitometry.

RNase Protection Assay

Total mRNA in the heart was prepared, and the amount of mRNA of Bcl-2 was determined by RNase protection assay using RPA III kit (Ambion). To probe Bcl-2, a partial fragment of mouse Bcl-2 gene was obtained by RT-PCR. A human 18S rRNA probe was used as an internal control. The relative intensity of Bcl-2 to 18S rRNA was quantified by densitometry.

Statistical Analysis

All data are reported as mean±SEM. Comparisons between AC5KO and WT values were made using Student's t-test. For statistical analysis of data from multiple groups, ANOVA was used. P<0.05 was taken as a minimal level of significance.

Results for Example 5: Distruption of Type 5 AC Did Not Affect the Development of Cardiac Hypertrophy

At baseline, there was no difference between WT and AC5KO in the LV weight (LVW; mg)/tibial length (TL; mm) (WT 4.7±0.2, AC5KO 5.1±0.2 mg/mm, n=9-14, P=NS). The time course and the degree of the development of cardiac hypertrophy (LVW/TL) in response to pressure overload were similar between WT and AC5KO (FIG. 1A). LVW/body weight, another index of cardiac hypertrophy, confirmed the data from LVW/TL (data not shown). Myocyte cross-sectional area, another index of hypertrophy, increased similarly in both WT and AC5KO at 3 weeks of banding, confirming the gross pathological data (FIG. 1B).

Cardiac Function Was Preserved in AC %^{-/-} After 3 Weeks of Aortic Banding

LV dimensions and cardiac function were evaluated echocardiographically. There was no difference in LVEDD and LVEF between WT and AC5KO at baseline and a 1 week after banding when they were compared to each other or to sham-operated animals (FIG. 2). At 3 weeks after banding, however, LVEDD was significantly increased in WT, while it remained unchanted in AC5KO (FIG. 2A). Similarly, LVEF fell significantly from 70±2.8 to 57±3.9% (P<0.05, n=8-11) in WT, while it remained unchanged at 74±2.2% in AC5KO (FIG. 2B). These results suggest that cardiac function was protected following chronic pressure overload in AC5KO. This was not due to a difference in pressure gradient, which was similar at 1 week after banding in AC5KO (102±8.2 mmHg) vs. WT (112±3.1 mmHg). Heart rate was not significantly different in WT and AC5KO under anesthesia during echocardiography, but was elevated in the conscious state in AC5KO (25).

Apoptosis Was Protected in AC5KO at 1 Week of Banding.

Before banding, there was no difference in the number of TUNEL-positive cells between the two groups, suggesting that the lack of type 5 AC did not alter the viability of cardiac myocytes at baseline. Aortic banding increased the number of TUNEL-positive cells in WT roughly 4-fold, at both 1 and 3 weeks after aortic banding (FIG. 3). The increase in apoptosis was roughly half that of WT at 3 weeks and less at 1 week after banding (FIG. 3).

Expression of Bcl-2 is Enhanced in AC5KO Hearts in Response to Pressure Overload

To examine changes in the molecules that are involved in apoptosis signaling, we quantitated Bel-2, an inhibitor of apoptosis, and Bax, an accelerator of apoptosis, in WT and AC5KO (FIG. 4). Bcl-2 expression was hardly detectable in the sham groups (data not shown). Interestingly, Bcl-2 protein expression was upregulated after 3 weeks of banding in both WT and AC5KO, although the magnitude of the increment was greater, P<0.05, in AC5KO (FIG. 4A). On the other hand, Bax expression was not different in the sham and banded groups (data not shown). We also examined the mRNA expression of Bcl-2. In parallel with Bcl-2 protein, mRNA of Bcl-2 was upregulated after 3 weeks of banding in both WT and AC5KO, but the magnitude of the increment was not different between WT and AC5KO (FIG. 4B). These results suggest that the apoptotic process is attenuated, at least in part, through the post-transcriptional regulation of Bcl-2 in AC5KO hearts.

### EXAMPLE 6

Motor Dysfunction in Type 5AC Null Mice

The neurotransmitter dopamine acts through various dopaminergic receptor subtypes that are associated with either stimulation or inhibition of adenylyl cyclases (AC), leading to the regulation of physiological functions such as the control of various motor functions or psychomotor activity. This dopamine-sensitive AC activity is highest in the striatum as well as in associated limbic structures of the brain, where their levels of activity by orders of magnitude exceed those in other areas of the brain. Such differences in striatal enzymatic activity may be attributed to the amount and/or combination of the enzyme isoforms that are expressed differentially in each brain region. The brain expresses all nine AC isoforms (AC1-AC9) that have distinct biochemical properties, i.e., regulation by Gi, Gβγ, calcium, or various kinases. Most, if not all, isoforms are enriched in specific brain region, rather than diffusely distributed throughout the brain. AC5, for example, is the dominant isoform in the striatum as well as in the heart. However, the coupling of each enzyme isoform to a specific neuronal function or functions, and a receptor signal remains unknown, as does whether the function of an AC isoform, unlike that of the receptors, can be substituted by another isoform.

The striatum is considered to be the center of sensorimotor integration within the basal ganglia (9) and receives widespread excitatory input from all regions of the cortex that converge with extensive dopaminergic, both D1 and D2, afferent from the midbrain. Concerted and balanced activity of these two dopaminergic signals is believed to play a key role in regulating striatal motor functions. In this study, we examined the role of their potential target enzyme isoform, AC5, by the use of knockout mice in which the AC5 gene was disrupted.

cAMP Production in Striatum Most Greatly Affected in AC5KO

To perform the experiments below, AC5KO mice were created. cAMP production in membranes from the striatum of AC5KO and WT at two months of age were examined. Results are shown in FIG. 7. AC activity was decreased in the striatum relative to that in WT by approximately 80% indicating that AC5 is the major isoform. In contrast, AC activity was significantly, but to a small degree, decreased in the cortext where where AC5 could be detected in WT and showed no difference in the cerebellum where AC was scarcely expressed in the WT. These findings confirmed the dominant expression of AC5 in the striatum, but not in the cortex or cerebellum. For comparison, AC activity in the heart was decreased in AC5KO by 30%-40%. Thus, the degree of contribution of AC5 to the total AC activity is greater in the striatum than in the heart.

AC5 and its Regulation of cAMP Signaling.

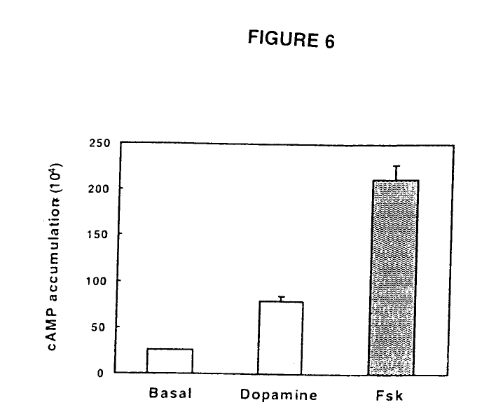

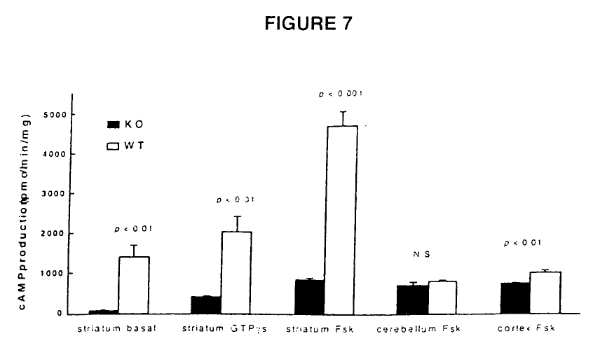

D1 agonist increases cAMP production while D2 agonist decreases it in Wild Type (“WT”). The effect of A2a agonist will be similar to that of D1 agonist. Results for WT are shown in FIG. 6. In AC5KO, both D1 and a2A agonists may increase cAMP production, but the absolute amount of increase may be smaller than that in WT, due to the decrease of total amount of AC expression in AC5KO.

Experimental Procedures

Rotor Rod Test

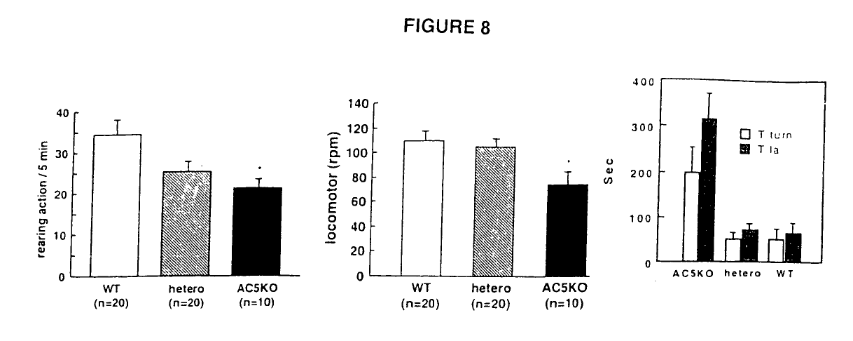

Rotor rod test, along with the rearing test (examines vertical movements), spontaneous movements test (examines horizontal movements), and pole test (examines the degree of bradykinesia) were used. Taken together these tests provide an index of spontaneous movement which can be used as an index of motor function. Most imporatntly, these tests were also used to evaluate the recovery of motor function upon cAMP administration and/or adenovirus mediated gene transfer of Ac5. Data were obtained from littermates of AC5KO, Hetero, and WT male. Horizontal and vertical spontaneous movements were not different between WT and Hetero, but were greatly reduced in AC5KO (FIG. 8, left and middle). Similarly, the pole test showed that the mice had an increased degree of bradykinesia (FIG. 8, right).

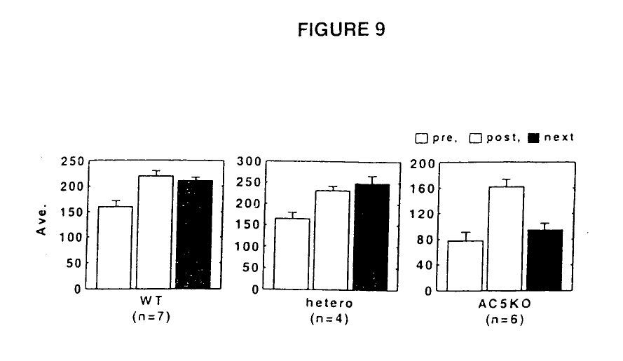

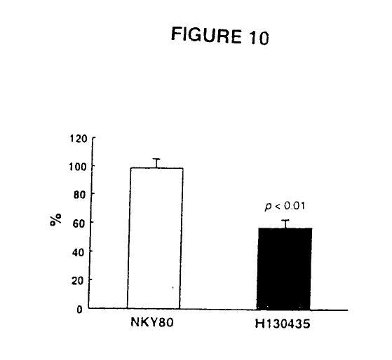

Motor function may be improved by the administration of cAMP analogues. The experiment demonstrated that the time that the mice spent on the rod without falling increased 30 minutes after db-cAMP injection; this was most likely a result from habituation or learning effect. However, the magnitude of improvements was greater in AC5KO (two-fold increase) than hetero and WT (25-30% increase). When the rotor rod performance was evaluated 48 hours after injection, the performance dropped to the basal level in AC5KO while remaining similar in Hetero and WT, which may be due to remaining habituation (FIG. 9).

RNase Protection Assay

Partial fragments of mouse AC cDNA clones for each isoform (types 1-9) and neutopeptides, i.e., enkephalin, substance P, dynorphin, were obtained by PCR. A human 28S ribosomal RNA probe was used as an internal control. RNase protection assay was performed using the RPA III kit (Ambion, Austin, Tex., USA).

AC assay

Striatal tissues were dissected from mice and membrane preparations were prepared for AC assays as previously described (10, 11).

Radioligand Binding Assay

D1 and D2 dopaminergic receptor binding assays were performed using ³H-SCH23390 and ³H-spiperone. Preliminary experiments demonstrated the Kd and Bmax values for D1 and D2 dopaminergic receptors were similar to those previously reported.

Behavioral Tests

Motor functions of mice were assessed by rotarod test (14), locomotor activity test, pole test, and tail suspension test.

Results

Impaired Motor Functions

Given that AC is the major effecter enzyme of dopaminergic signals in the striatum, we conducted various motor function tests to evaluate striatal function in an animal model in which the AC5 gene was disrupted (AC5KO). The most dramatic change was found in coordinated movement, which was evaluated by rotarod performance. In this test, we measured the time that mice could stay on an accelerating roltarod without falling. In general, there was a major impairment in AC5KO and to a lesser degree in the heterozygous mice relative to WT (FIG. 2A). AC5KO could spend significantly less time on the rotarod and heterozygous mice slightly less than WT. When tests were repeated, their performance improved significantly after a few trials. However, AC 5KO performed very poorly even after several trials. When the test was repeated on the following day, the results were similar, disputing the possibility that AC5KO required a longer period to learn the performance (data not shown).

Spontaneous activity was determined both horizontally (locomotion) and vertically (rearings). Mice were placed in a cage and their movements were videotaped for analysis. WT and heterozygous mice revealed a similar performance in open field locomotor activity while AC5KO showed a small, but significant degree of reduction (FIGS. 2B and 2C). In order to evaluate bradykinesia, pole test was performed. The time until they turned downward (Tturn) and the time until they descended to the floor were measured (TLA). We found that AC5KO had marked deficits in this test; they showed an over 3-fold prolongation of both recording time indexes (FIG. 2D). It was also possible that striatal dysfunction led to choleric or dystonic movements. Such abnormal movements may be demonstrated most readily in mice as a clasping of the limbs that is triggered by tail suspension test (17). However, we found no such abnormal movements in both AC5KO and WT (data not shown).

AC5 Expression and AC Activity in AC5KO

These results indicated impairments of striatal functions in AC5KO, presumably induced by the loss of AC5. While AC5 may be striatum-specific with regard to its distribution (18), it remained unknown whether it was dominant for cAMP production in the striatum. AC5 mRNA was expressed at least 10-20 fold more abundantly in the striatum than in the other brain regions, such as the cortex and the cerebellum, in WT (FIG. 3A); this was in agreement with previous findings (18). In AC5KO, AC5 mRNA expression was negated, but histological examinations revealed no changes such as neuronal loss and/or reactive gliosis at 8 to 12 weeks-old (data not shown). We found, however, that AC activity was greatly decreased in striatal membrane preparations in AC5KO (FIG. 3B). In contrast, AC activity was significantly, but only to a small degree, decreased in the cortex where AC5 could be detected in WT, and showed no difference in the cerebellum where AC5 was scarcely detected in WT. For comparison, AC activity in the heart, another tissue in which AC5 is dominantly expressed (8), was decreased by only 30% (data not shown), indicating that the contribution of AC5 to cAMP production is greater in the striatum than in the heart.

We also examined receptor agonist-stimulated AC activity (FIG. 3C). In many tissues including the heart, in general, marked stimulation of AC is readily attainable with Gs-coupled receptor agonists although the inhibition of AC with Gi-coupled receptor agonists may not be always easy. In the striatum, however, SKF38393, a D1 dopaminergic receptor agonist, modestly stimulated AC activity in WT (40.7±2.6% increase over that with 10 μM GTP). Quinpirole, a D2 agonist, inhibited SKF38393-stimulated AC activity; the inhibition was significant but small (13.5±1.1% decrease). In AC5KO, the responses to D1 and D2 dopaminergic receptor agonists were markedly diminished; the D1 dopaminergic agonist-mediated stimulation was very small and the D2 dopaminergic agonist-mediated inhibition in AC5KO was hardly detectable. It is tentative to speculate that the loss of D2 agonist-mediated inhibition was due to the loss of AC5, which is Gi-inhibitable, as proposed recently in a similar model (20), while it is also possible that the AC catalytic activity was too low to demonstrate inhibition by AC assays with membrane preparations. Thus, in vitro AC assays may not be sufficient in terms of sensitivity to study changes in selective dopaminergic signal in AC5KO. We did not understand, however, why the response to D1 agonist stimulation as shown by percent increase was also attenuated in AC5KO because other remnant AC isoforms must be able to respond to Gs, if not Gi, stimulation. We also examined cAMP accumulation in intact striatal neuronal cells from the fetus; however, the difference in cAMP production was not as great as in the above AC assays using membrane preparations from adults (data not shown).

Changes in Other Molecules Involved in Dopaminergic Signal

The disruption of the major striatal AC isoform may change the expression of other molecules involved in dopaminergic signaling. D1 dopaminergic receptor binding sites were modestly decreased in AC5KO while D2 receptor binding sites were similar to those in WT (FIG. 4A). The short, but not the long, form of Gs protein expression was decreased in AC5KO (FIG. 4B), presumably due to the loss of positive feed forward regulatory loop. Western blotting for various molecules, using either the membrane preparation or whole tissue homogenates, revealed that the protein expression of Golf, Gi, Gq, Gβ, and PKA (the alpha catalytic subunit) were not changed (data not shown). Changes in neurotransmitters, such as dynorphin, substance P, and enkephalin, were examined by RNase protection assays that may be linked to the activity of D1 and D2 dopaminergic receptors. The expression of dynorphin, which acts on presynaptic k-receptors to inhibit AC (20), was modestly increased (FIG. 4C). In contrast, the expressions of enkephalin and substance P were unchanged (FIG. 4C). The expressions of glutamic acid decarboxylase and tyrosine hydroxylase, which are involved in the synthesis of gamma aminobutyric acid and dopamine respectively, were also unchanged as determined by immunoblotting (data not shown). The above findings suggested that the expression of some molecules, i.e., D1 dopaminergic receptors, Gs, and dynorphin, was changed in such a way to suppress the D1 dopaminergic pathway despite the disruption of the major AC isoform.

We then examined if there was any increase in the expression of other AC isforms in AC5KO. Since AC isoform antibodies that can convincingly determine the level of protein expression are not available, we quantitated the mRNA expression of the AC isoforms by RNase protection assays (FIG. 4D). All AC isoforms except AC4 and AC8 could be detected. Among these isoforms, we found a modest increase of AC6, the most relevant isoform to AC5, as well as AC2, AC7, and AC9, but not AC1 and AC3 in AC5KO. We thought, however, that such small increases in the expression of AC isoforms were not sufficient to explain decreases in the expression of Gs and D1 dopaminergic receptors, which occurred as if to inhibit D1 dopaminergic pathway.

Dopaminergic Agonists Improved Motor Function in ACD^{-/-}

If either or both D1 and D2 dopaminergic signals were attenuated in AC5KO leading to motor dysfunction in vivo, stimulation of dopaminergic receptors, D1 and/or D2, with specific agonists may restore the function. Administration of SKF38393 (25 or 50 mg/kg), a D1 dopaminergic agonist, increased locomotor activity in both WT and AC5KO. In particular, locomotor activity response appeared pronounced, and might be supersensitive, in AC5KO relative to WT (FIG. 5A). This finding was reminiscent of the supersensitive response of the direct pathway neurons observed in dopamine depletion of Parkinson's disease, in which the D1 dopaminergic function becomes supersensitive but is accompanied by an actual reduction of D1 dopamine receptor levels (21, 22) (FIG. 4A). SKF38393 did not improve rotarod performance in both WT and AC5KO (FIG. 5B). We then examined the effect of cabergoline (0.2 or 1.0 mg/kg), a D2 dopaminergic agonist, which has been used in the treatment of Parkinson's diseases. Cabergoline had no significant effect on locomotor activity in both WT and AC5KO although both showed a tendency of small increases (FIG. 5D). In contrast, cabergoline improved rotarod performance selectively in AC5KO; their performance indeed reached an equivalent level to that of WT (FIG. 5E) while cabergoline essentially had no effect on WT, suggesting that coordination in AC5KO was restored by D2 dopaminergic stimulation. We also examined the effect of these agonists on pole test performance (FIGS. 5C and 5F). Both SKF38393 and cabergoline improved pole test performance in AC5KO, the latter of which induced a dramatic improvement even with a lower dose (0.2 mg/kg).

Discussion

We have demonstrated that the disruption of the AC5 gene led to a major deficit in AC activity in a striatal specific manner and an abnormal coordination represented by impaired rotarod performance as well as other motor disorders, which mimicked Parkinson's disease. Selective stimulation of D2 dopaminergic receptors by cabergoline restored coordination, suggesting that the attenuation of D2 dopaminergic signal underlied abnormal coordination in AC5KO, and that D2 dopaminergic signal targets AC5 as a major effecter isoform. Locomotor activity was also attenuated and restored by selective D1 dopaminergic stimulation, suggesting that this dopaminergic signal also targets AC5. In contrast, both dopaminergic signals may be able to couple to other AC isoforms as well because D1 or D2 dopaminergic stimulation could restore specific motor function, i.e. coordination or locomotion, respectively. Nevertheless, such selective dopaminergic agonist stimulation could not restore all of the motor disorders, indicating that AC5 is essential in balancing and maintaining both coordination and locomotion, and may provide the site of convergence of both D1 and D2 dopaminergic signals. D1 and D2 are the most abundant dopaminergic receptors expressed in he brain, and both are involved in the two major striatal output pathways, i.e., the “direct” and the “indirect” pathways, which are dominantly mediated by D1 and D2 receptors, respectively. Although it is still unknown whether these receptor subtypes are expressed in the distinct populations of striatal neurons (23) or within the same populations (24), it has been believed that the parallel and balanced activation of these two pathways and their synergistic action control striatal motor functions. Our findings indicate that the parallel and balanced activation are maintained by the presence of AC5 that is coupled to both dopaminergic pathways. In the absence of AC5, AC6 and AC1 are still present, but can not fully compensate for the function of AC5.

The supersensitive response to D1 dopaminergic stimulation (FIG. 5A) mimicked the supersensitivity in Parkinson's disease. Alike Parkinson's disease, AC5KO also had decreased D1 dopaminergic receptor expression (21, 22). Because there was no upregulation of G protein or PKA expression, changes responsible for this supersensitization must be located in the downstream of PKA although we do not deny that compensation in the AC pathway could include increased translation and/or post translation activation of remaining AC isoforms or other pathway components. The exact molecular mechanisms for this paradoxical phenomenon have remained unexplained also in Parkinson's disease, but a very recent study suggested that a switch in the regulation of downstream MAPK signal may be involved (25). The dopamine depletion in Parkinson's disease and the lack of its major effecter enzyme isoform may be similar in many aspects and thus AC5KO may be useful to explore the molecular mechanisms for supersensitization in future studies.

The following References, cited in Example 1 are hereby icorprated in their entirety.
1. Murray, K. J., Reeves, M. L., and England, P. J. 1989. Protein-phosphorylation and compartments of cyclic AMP in the control of cardiac contraction. Mol. Cell. Biochem. 89:175-179.
2. Federman, A. D., Conklin, B. R., Schrader, K. A., Reed, R. R., and Bourne, H. R. 1992. Hormonal stimulation of adenylyl cyclase through Gi-protein beta gamma subunits. Nature 356:159-161.
3. Ishikawa, Y. 1998. Regulation of cAMP signaling by phosphorylation. Adv. Second Messenger Phosphoprotein Res. 32:99-120.
4. Hanoune, J., and Defer, N. 2001. Regulation and role of adenylyl cyclase isoforms. Annu. Rev. Pharmacol. Toxicol. 41:145-174.
5. Caulfield, M. P. 1993. Muscarinic receptors—characterization, coupling and function. Pharmacol. Ther. 58:319-379.
6. Yatani, A., Codina, J., Brown, A. M., and Birnbaumer, L. 1987. Direct activation of mammalian atrial muscarinic potassium channels by GTP regulatory protein Gk. Science 235:207-211.
7. Wickman, K., Nemec, J., Gendler, S. J., and Clapham, D. E. 1998. Abnormal heart rate regulation in GIRK4 knockout mice. Neuron 20:103-114.
8. Ishikawa, Y., and Homcy, C. J. 1997. The adenylyl cyclases as integrators of transmembrane signal transduction. Circ. Res. 80:297-304.
9. Tang, W. J., and Hurley, J. H. 1998. Catalytic mechanism and regulation of mammalian adenylyl cyclases. Mol. Pharmacol. 54:231-240.
10. Hurley, J. H. 1999. Structure, mechanism, and regulation of mammalian adenylyl cyclase. J. Biol. Chem. 274:7599-7602.
11. Tobise, K., Ishikawa, Y., Holmer, S. R., Im, M. J., Newell, J. B., Yoshie, H., Fujita, M., Susannie, E. E., and Homcy, C. J. 1994. Changes in type VI adenylyl cyclase isoform expression correlate with a decreased capacity for cAMP generation in the aging ventricle. Circ. Res. 74:596-603.
12. Espinasse, I., Iourgenko, V., Defer, N., Samson, F., Hanoune, J., and Mercadier, J. J. 1995. Type V, but not type VI, adenylyl cyclase mRNA accumulates in the rat heart during ontogenic development. Correlation with increased global adenylyl cyclase activity. J. Mol. Cell. Cardiol. 27:1789-1795.
13. Taussig, R., Iniguez-Lluhi, J. A., and Gilman, A. G. 1993. Inhibition of adenylyl cyclase by Gi alpha. Science 261:218-221.
14. Dessauer, C. W., Tesmer, J. J., Sprang, S. R., and Gilman, A. G. 1998. Identification of a Gialpha binding site on type V adenylyl cyclase. J. Biol. Chem. 273:25831-25839.
15. Vatner, S. F., and Braunwald, E. 1972. Cardiac frequency: control and adjustments to alterations. Prog. Cardiovasc. Dis. 14:431-445.
16. Higgins, C. B., Vatner, S. F., and Braunwald, E. 1973. Parasympathetic control of the heart. Pharmacol. Rev. 25:119-155.
17. Ishikawa, Y., Sorota, S., Kiuchi, K., Shannon, R. P., Komamura, K., Katsushika, S., Vatner, D. E., Vatner, S. F., and Homcy, C. J. 1994. Downregulation of adenylylcyclase types V and VI mRNA levels in pacing-induced heart failure in dogs. J. Clin. Invest. 93:2224-2229.
18. Bradford, M. M. 1976. A rapid and sensitive method for the quantitation of microgram quantities of protein utilizing the principle of protein-dye binding. Anal. Biochem. 72:248-254.
19. Kawabe, J. -i., Iwami, G., Ebina, T., Ohno, S., Katada, T., Ueda, Y., Homcy, C. J., and Ishikawa, Y. 1994. Differential activation of adenylyl cyclase by protein kinase C isoenzymes. J. Biol. Chem. 269:16554-16558.
20. Kawabe, J. -i., Aizawa, Y., Takehara, N., Hasebe, N., and Kikuch, K. 2000. Glucose modifies the cross-talk between insulin and the beta-adrenergic signaling system in vascular smooth muscle cells. J. Hypertens. 18:1457-1464.
21. Iwase, M., Uechi, M., Vatner, D. E., Asai, K., Shannon, R. P., Kudej, R. K., Wagner, T. E., Wight, D. C., Patrick, T. A., Ishikawa, Y., et al. 1997. Cardiomyopathy induced by cardiac Gs alpha overexpression. Am. J. Physiol. 272:H585-589.
22. Uechi, M., Asai, K., Osaka, M., Smith, A., Sato, N., Wagner, T. E., Ishikawa, Y., Hayakawa, H., Vatner, D. E., Shannon, R. P., et al. 1998. Depressed heart rate variability and arterial baroreflex in conscious transgenic mice with overexpression of cardiac Gsalpha. Circ. Res. 82:416-423.
23. Homcy, C. J., Vatner, S. F., and Vatner, D. E. 1991. Beta-adrenergic receptor regulation in the heart in pathophysiologic states: abnormal adrenergic responsiveness in cardiac disease. Annu. Rev. Physiol. 53:137-159.
24. Tepe, N. M., Lorenz, J. N., Yatani, A., Dash, R., Kranias, E. G., Dorn, G. W., 2nd, and Liggett, S. B. 1999. Altering the receptor-effector ratio by transgenic overexpression of type V adenylyl cyclase: enhanced basal catalytic activity and function without increased cardiomyocyte beta-adrenergic signalling. Biochemistry 38:16706-16713.
25. Gao, M. H., Lai, N. C., Roth, D. M., Zhou, J., Zhu, J., Anzai, T., Dalton, N., and Hammond, H. K. 1999. Adenylylcyclase increases responsiveness to catecholamine stimulation in transgenic mice. Circulation 99:1618-1622.
26. Lipskaia, L., Defer, N., Esposito, G., Hajar, I., Garel, M. C., Rockman, H. A., and Hanoune, J. 2000. Enhanced cardiac function in transgenic mice expressing a Ca(2+)-stimulated adenylyl cyclase. Circ. Res. 86:795-801.
27. Wu, Z. L., Thomas, S. A., Villacres, E. C., Xia, Z., Simmons, M. L., Chavkin, C., Palmiter, R. D., and Storm, D. R. 1995. Altered behavior and long-term potentiation in type I adenylyl cyclase mutant mice. Proc. Natl. Acad. Sci. USA 92:220-224.
28. Wong, S. T., Trinh, K., Hacker, B., Chan, G. C., Lowe, G., Gaggar, A., Xia, Z., Gold, G. H., and Storm, D. R. 2000. Disruption of the type III adenylyl cyclase gene leads to peripheral and behavioral anosmia in transgenic mice. Neuron 27:487-497.
29. Watson, E. L., Jacobson, K. L., Singh, J. C., Idzerda, R., Ott, S. M., DiJulio, D. H. Wong, S. T., and Storm, D. R. 2000. The type 8 adenylyl cyclase is critical for Ca2+ stimulation of cAMP accumulation in mouse parotid acini. J. Biol. Chem. 275:14691-14699.
30. Wickman, K., and Clapham, D. E. 1995. Ion channel regulation by G proteins. Physiol. Rev. 75:865-b885.

1-38. (canceled)

39. A pharmaceutical composition comprising a pharmaceutically acceptable carrier and at least one compound of the formula 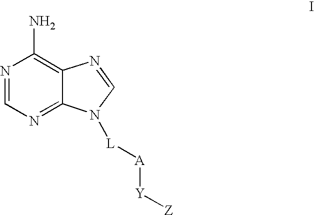
wherein A is a direct link or A is a divalent member selected from the group consisting of:
phenyl, thienyl, furanyl, pyrrolyl, indolyl, 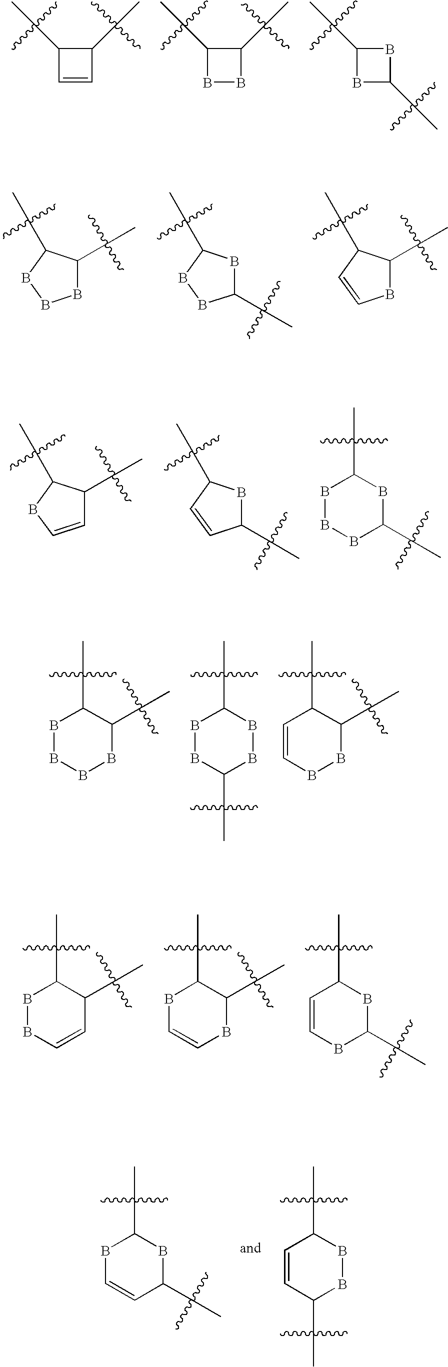
wherein
each B is independently —C(—R¹)(—R²)—, —O— or —N(-J-R³)—, and wherein only one ring B is either O or —N(-J-R³)—;
m and n are each independently an integer from 0-4;
q is an integer from 0 to 8;
Y is —(CH₂)_{q}—, —(CH₂)ₘO—, —(CH₂)ₘ—N(-J¹-)-R⁴;
Z is —(CH₂)ₙ—C(═O)—NHOH and —(CH₂)ₙCOOH;
L is —(CH₂)_{q}—, —(CH₂)ₘO—, —(CH₂)ₘ—N—(-J²) -R⁵;
J, J¹ and J² are each independently —C(═O)— or a direct link;
R¹ is H, —N(-J³-R⁶)(-J⁴-R⁷) or —O-J⁵-R⁸;
wherein J³, J⁴ and J⁵ are each independently —C(═O)—, a direct link, or at least one of J³ and J⁴ is a direct link;
R² is H, —N(-J⁶-R⁹)(-J⁷-R¹⁰) or —O-J⁸-R¹¹;
wherein J⁶, J⁷ and J⁸ are each independently —C(═O)—, a direct link, or at least one of J⁶ and J⁷ is a direct link;
R³ is H, C₁-C₈ alkyl, CF₃, or —O—R¹²;
R⁴ is H, C₁-C₈ alkyl, CF₃, or —O—R¹³;
R⁵ is H, C₁-C₈ alkyl, CF₃, or —O—R¹⁴;
R⁶ is H, C₁-C₈ alkyl, CF₃, or —O—R¹⁵;
R⁷ is H, C₁-C₈ alkyl, CF₃, or —O—R¹⁶;
R⁸ is H, C₁-C₈ alkyl, CF₃, or —O—R¹⁷;
R⁹ is H, C₁-C₈ alkyl, CF₃, or —O—R¹⁸;
R¹⁰ is H, C₁-C₈ alkyl, CF₃, or —O—R¹⁹;
R¹¹ is H, C₁-C₈ alkyl, CF₃, or —O—R²⁰;
R¹², R ¹³, R¹⁴, R¹⁵, R ¹⁶, R¹⁷, R¹⁸, R¹⁹ and R²⁰ are each independently C₁-C₄ alkyl, cycloalkyl or benzyl

40. A method of treating neurodegenerative diseases, said method, comprising administering a pharmaceutically effective amount of at least one compound capable of regulating AC5 to a patient in need of treatment.

41. A method of treating a cardiovascular disease or condition, comprising administering a pharmaceutically effective amount of at least one compound capable of regulating AC5 to a patient in need of treatment.

42. The method of claim 40, wherein the at least one compound has the formula 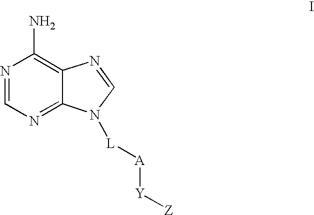
wherein A is a direct link or A is divalent member selected from the group consisting of:
phenyl, thienyl, furanyl, pyrrolyl, indolyl,
wherein
each B is independently —C(—R¹)(—R²)—, —O— or —N(-J-R³)—, and wherein only one ring B is either O or —N(-J-R³)—;
m and n are each independently an integer from 0-4;
q is an integer from 0 to 8;
Y is —(CH₂)_{q}—, —(CH₂)ₘO—, —(CH₂)ₘ—N(-J¹-)-R⁴;
Z is —(CH₂)ₙ—C(═O)—NHOH and —(CH₂)ₙCOOH;
L is —(CH₂)_{q}—, —(CH₂)ₘO—, —(CH₂)ₘ—N—(-J²-)-R⁵;
J, J¹ and J² are each independently —C(═O)— or a direct link;
R¹ is H, —N(-J³-R⁶)(-J⁴-R⁷) or —O-J⁵-R⁸;
wherein J³, J⁴ and J⁵ are each independently —C(═O)—, a direct link, or at least one of J³ and J⁴ is a direct link;
R² is H, —N(-J⁶-R⁹)(J⁷-R¹⁰) or —O-J⁸-R¹¹;
wherein J⁶, J⁷ and J⁸ are each independently —C(═O)—, a direct link, or at least one of J⁵ and J⁷ is a direct link;
R³ is H, C₁-C₈ alkyl, CF₃, or —O—R¹²;
R⁴ is H, C₁-C₈ alkyl, CF₃, or —O—R¹³;
R⁵ is H, C₁-C₈ alkyl, CF₃, or —O—R¹⁴;
R⁶ is H, C₁-C₈ alkyl, CF₃, or —O—R¹⁵;
R⁷ is H, C₁-C₈ alkyl, CF₃, or —O—R¹⁶;
R⁸ is H, C₁-C₈ alkyl, CF₃, or —O—R¹⁷;
R⁹ is H, C₁-C₈ alkyl, CF₃, or —O—R¹⁸;
R¹⁰ is H, C₁-C₈ alkyl, CF₃, or —O—R¹⁹;
R¹¹ is H, C₁-C₈ alkyl, CF₃, or —O—R²⁰;
R¹², R¹³, R¹⁴, R¹⁵, R¹⁶, R¹⁷, R¹⁸, R¹⁹ and R²⁰ are each independently C₁-C₄ alkyl, cycloalkyl or benzyl;
and all pharmaceutically acceptable isomers, salts, hydrates, solvates and prodrug derivatives thereof.

43. A method of treating a patient for loss of motor function comprising administering at least one compound capable of regulating AC5.

44. The method of claim 40, wherein said patient is in need of treatment of decreased motor function.

45. The method of claim 40, wherein said patient is in need of treatment of impaired motor function.

46. The method of claim 1, wherein said patient is in need of treatment of a disease or condition selected from the group consisting of Parkinson's Disease, Huntington's disease, Alzheimer's disease, stroke, and dementia.

47. The method of claim 40 wherein the regulating is stimulation of AC5 activity.

48. The method of claim 40 wherein the regulating is inhibition of AC5 activity.

49. The method of claim 40, wherein the at least one compound is selected from compounds of formula: 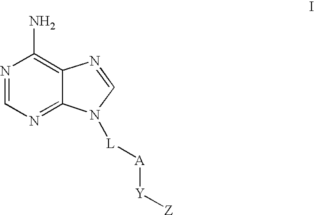
wherein
A is a direct link or A is divalent member selected from the group consisting of:
phenyl, thienyl, furanyl, pyrrolyl, indolyl, 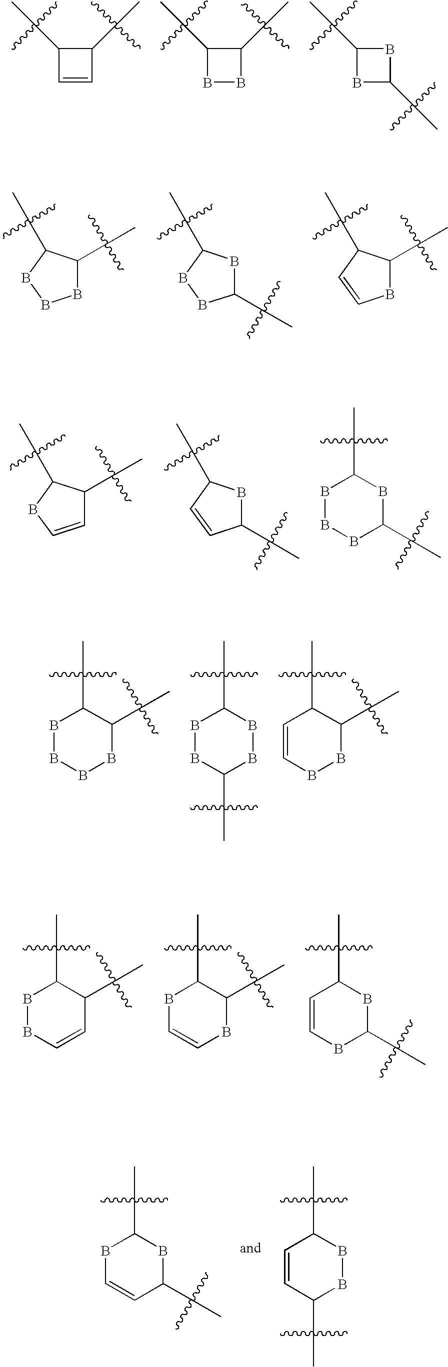 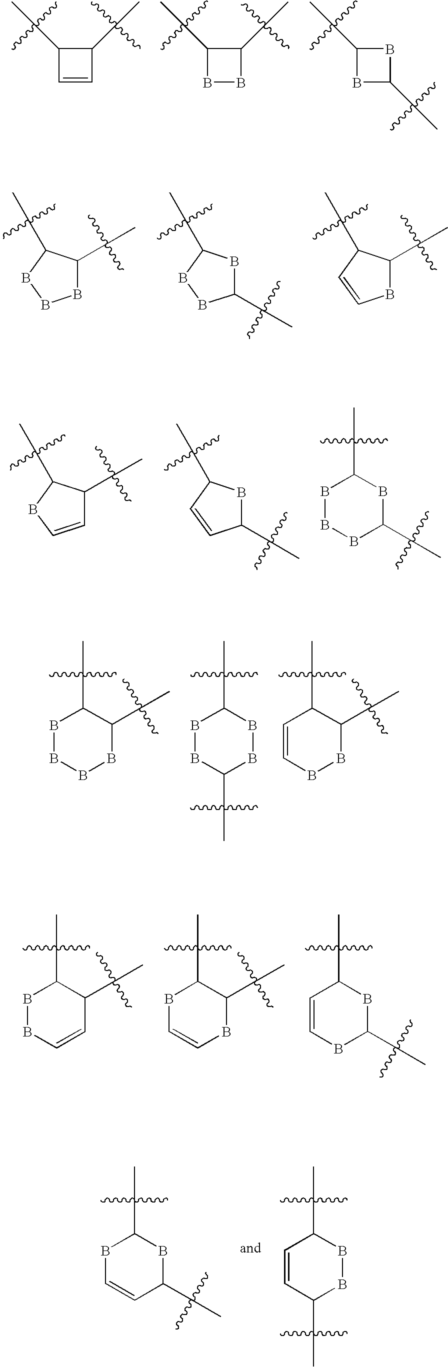
wherein
each B is independently —C(—R¹)(—R²)—, —O— or —N(-J-R³)—, and wherein only one ring B is either O or —N(-J-R³)—;
m and n are each independently an integer from 0-4;
q is an integer from 0 to 8;
Y is —(CH₂)_{q}—, —(CH₂)ₘO—, —(CH₂)ₘ—N(-J¹-)-R⁴;
Z is —(CH₂)ₙ—C(═O)—NHOH and —(CH₂)ₙCOOH;
L is —(CH₂)_{q}—, —(CH₂)ₘO—, —(CH₂)ₘ—N-(-J²-)-R⁵;
J, J¹ and J² are each independently —C(═O)— or a direct link;
R¹ is H, —N(-J³-R⁶)(J⁴-R⁷) or —O-J⁵-R⁸;
wherein J³, J⁴ and J⁵ are each independently —C(═O)—, a direct link, or at least one of J³ and J⁴ is a direct link;
R² is H, —N(-J⁶-R⁹)(-J⁷-R¹⁰) or —O-J⁸-R¹¹;
wherein J⁶, J⁷ and J⁸ are each independently —C(═O)—, a direct link, or at least one of J⁶ and J⁷ is a direct link;
R³ is H, C₁-C₈ alkyl, CF₃, or —O—R¹²;
R⁴ is H, C₁-C₈ alkyl, CF₃, or —O—R¹³;
R⁵ is H, C₁-C₈ alkyl, CF₃, or —O—R¹⁴;
R⁶ is H, C₁-C₈ alkyl, CF₃, or —O—R¹⁵;
R⁷ is H, C₁-C₈ alkyl, CF₃, or 1'O—R¹⁶;
R⁸ is H, C₁-C₈ alkyl, CF₃, or —O—R¹⁷;
R⁹ is H, C₁-C₈ alkyl, CF₃, or —O—R¹⁸;
R¹⁰ is H, C₁-C₈ alkyl, CF₃, or —O—R¹⁹;
R¹¹ is H, C₁-C₈ alkyl, CF₃, or —O—R²⁰;
R¹², R¹³, R¹⁴, R¹⁵, R¹⁶, R¹⁷, R¹⁸, R¹⁹ and R²⁰ are each independently C₁-C₄ alkyl, cycloalkyl or benzyl

50. The method of claim 40, wherein said patient is human.

51. A method of treating motor function complications after cerebrovascular disease, said method comprising administering a pharmaceutically effective amount of at least one compound capable of regulating AC5 activity to a patient in need of treatment.

52. The method of claim 41, wherein the at least one compounds is selected from compounds of formula: 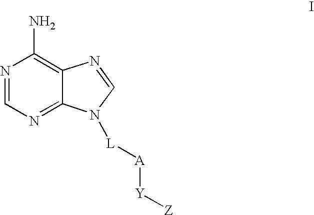
wherein A is a direct link or A is divalent member selected from the group consisting of:
phenyl, thienyl, furanyl, pyrrolyl, indolyl, 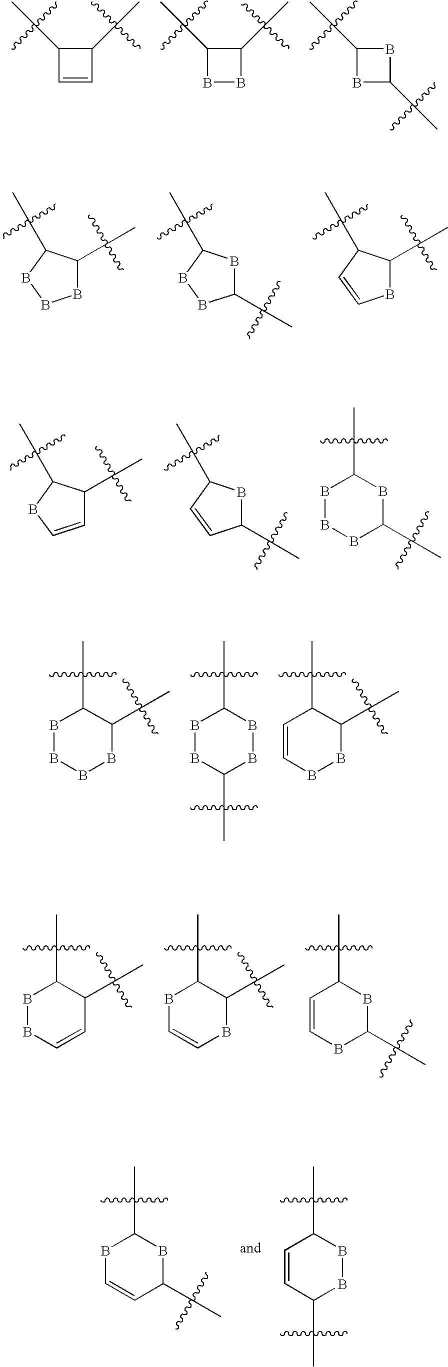
wherein
each B is independently —C(—R¹)(—R²)1', —O— or —N(-J-R³)—, and wherein only one ring B is either O or —N(-J-R³)—;
m and n are each independently an integer from 0-4;
q is an integer from 0 to 8;
Y is —(CH₂)_{q}—, —(CH₂)ₘO—, —(CH₂)ₘ—N(-J¹-)-R⁴;
Z is —(CH₂)ₙ—C(═O)—NHOH and —(CH₂)ₙCOOH;
L is —(CH₂)_{q}—, —(CH₂)ₘO—, —(CH₂)ₘ—N-(J²) -R⁵;
J, J¹ and J² are each independently —C(═O)— or a direct link;
R¹ is H, —N(-J³-R⁶)(-J⁴-R⁷) or —O-J⁵-R⁸;
wherein J³, J⁴ and J⁵ are each independently —C(═O)—, a direct link, or at least one of J³ and J⁴ is a direct link;
R² is H, —N(-J⁶-R⁹)(-J⁷-R¹⁰) or —O-J⁸-R¹¹;
wherein J⁶, J⁷ and J⁸ are each independently —C(═O)—, a direct link, or at least one of J⁶ and J⁷ is a direct link;
R³ is H, C₁-C₈ alkyl, CF₃, or —O—R¹²;
R⁴ is H, C₁-C₈ alkyl, CF₃, or —O—R¹³;
R⁵ is H, C₁-C₈ alkyl, CF₃, or —O1'R¹⁴;
R⁶ is H, C₁-C₈ alkyl, CF₃, or —O1'R¹⁵;
R⁷ is H, C₁-C₈ alkyl, CF₃, or —O—R¹⁶;
R⁸ is H, C₁-C₈ alkyl, CF₃, or —O—R¹⁷;
R⁹ is H, C₁-C₈ alkyl, CF₃, or —O—R¹⁸;
R¹⁰ is H, C₁-C₈ alkyl, CF₃, or —O—R¹⁹;
R¹¹ is H, C₁-C₈ alkyl, CF₃, or —O—R²⁰;
R¹², R¹³, R¹⁴, R¹⁵, R¹⁶, R¹⁷, R¹⁸, R¹⁹ and R²⁰ are each independently C₁-C₄ alkyl, cycloalkyl or benzyl

53. The method of claim 41 wherein the regulating is inhibition of AC5 activity.

54. A method of treating neuronal infection, said method comprising administering a pharmaceutically effective amount of at least one compound capable of regulating AC5 inhibitor to a patient in need of treatment.

55. The method of claim 54, wherein the at least one compound is selected from compounds of formula: 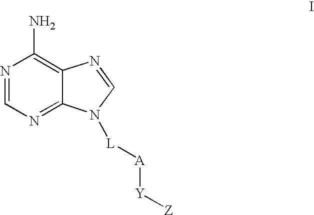
wherein A is a direct link or A is divalent member selected from the group consisting of:
phenyl, thienyl, furanyl, pyrrolyl, indolyl, 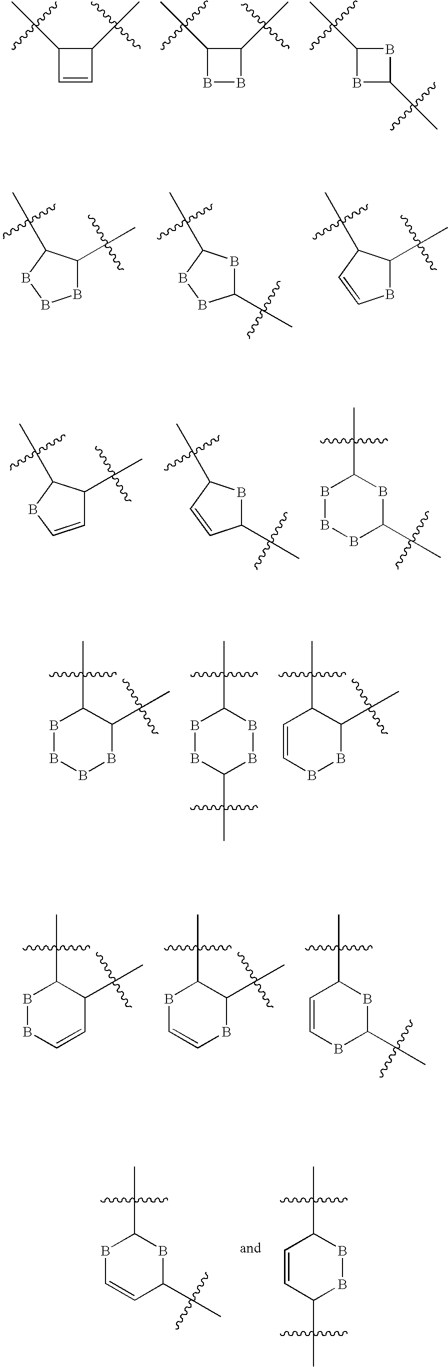
wherein
each B is independently —C(—R¹)(—R²)—,—O— or —N(-J-R³)—, and wherein only one ring B is either O or —N(-J-R³)—;
m and n are each independently an integer from 0-4;
q is an integer from 0 to 8;
Y is —(CH₂)_{q}—, —(CH₂)ₘO—, —(CH₂)ₘ—N(-J¹-)-R⁴;
Z is —(CH₂)ₙ—C(═O)—NHOH and —(CH₂)ₙCOOH;
L is —(CH₂)_{q}—, —(CH₂)ₘO—, —(CH₂)ₘ—N-(J²-)-R⁵;
J, J¹ and J² are each independently —C(═O)— or a direct link;
R¹ is H, —N(-J³-R⁶)(-J⁴-R⁷) or —O-J⁵-R⁸;
wherein J³, J⁴ and J⁵ are each independently —C(═O)—, a direct link, or at least one of J³ and J⁴ is a direct link;
R² is H, —N(-J⁶-R⁹)(J⁷-R¹⁰) or —O-J⁸-R¹¹;
wherein J⁶, J⁷ and J⁸ are each independently —C(═O)—, a direct link, or at least one of J⁶ and J⁷ is a direct link;
R³ is H, C₁-C₆ alkyl, CF₃, or —O—R¹²;
R⁴ is H, C₁-C₈ alkyl, CF₃, or —O—R¹³;
R⁵ is H, C₁-C₈ alkyl, CF₃, or —O—R¹⁴;
R⁵ is H, C₁-C₈ alkyl, CF₃, or —O—R¹⁵;
R⁷ is H, C₁-C₈ alkyl, CF₃, or —O—R¹⁶;
R⁸ is H, C₁-C₈ alkyl, CF₃, or —O—R¹⁷;
R⁹ is H, C₁-C₈ alkyl, CF₃, or —O—R¹⁸;
R¹⁰ is H, C₁-C₈ alkyl, CF₃, or —O—R¹⁹;
R¹¹ is H, C₁-C₈ alkyl, CF₃, or —O—R²⁰;
R¹², R¹³, R¹⁴, R¹⁵, R¹⁶, R¹⁷, R¹⁸, R¹⁹ and R²⁰ are each independently C₁-C₄ alkyl, cycloalkyl or benzyl;
and all pharmaceutically acceptable isomers, salts, hydrates, solvates and prodrug derivatives thereof.

54. The method of claim 43, wherein said patient is in need of treatment of secondary motor function complications following encephalitis.

55. The method of claim 43, wherein said patient is in need of treatment of secondary motor function complications following West Nile virus.

56. The method of claim 43, wherein the at least one compounds is selected from compounds of formula: 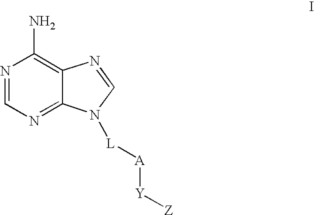
wherein A is a direct link or A is divalent member selected from the group consisting of:
phenyl, thienyl, furanyl, pyrrolyl, indolyl, 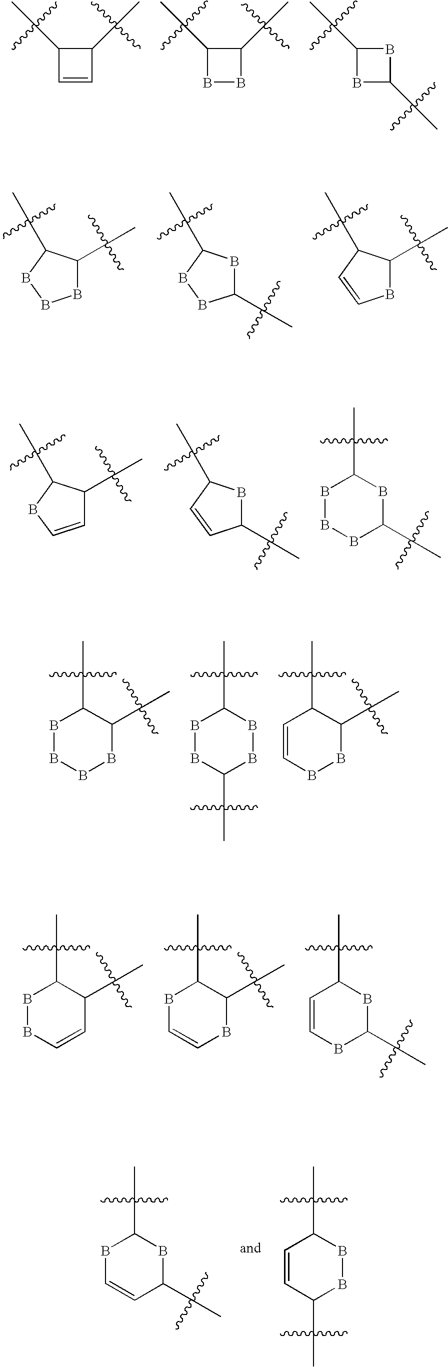
wherein
each B is independently —C(—R¹)(—R²)—, —O— or -N(-J-R³)—, and wherein only one ring B is either O or —N(-J-R³)—;
m and n are each independently an integer from 0-4;
q is an integer from 0 to 8;
Y is —(CH₂)_{q}—, —(CH₂)ₘO—, —(CH₂)ₘ—N(-J¹-)-R⁴;
Z is —(CH₂)ₙ—C(═O)—NHOH and —(CH₂)ₙCOOH;
L is —(CH₂)_{q}—, —(CH₂)ₘO—, —(CH₂)ₘ—N-(J²-)-R⁵;
J, J¹ and J² are each independently —C(═O)— or a direct link;
R¹ is H, —N(-J³-R⁶)(-J⁴-R⁷) or —O-J⁵-R⁸;
wherein J³, J⁴ and J⁵ are each independently —C(═O)—, a direct link, or at least one of J³ and J⁴ is a direct link;
R² is H, —N(-J⁶-R⁹)(J⁷-R¹⁰) or —O-J⁸-R¹¹;
wherein J⁶, J⁷ and J⁸ are each independently —C(═O)—, a direct link, or at least one of J⁶ and J⁷ is a direct link;
R³ is H, C₁-C₆ alkyl, CF₃, or —O—R¹²;
R⁴ is H, C₁-C₈ alkyl, CF₃, or —O—R¹³;
R⁵ is H, C₁-C₈ alkyl, CF₃, or —O—R¹⁴;
R⁵ is H, C₁-C₈ alkyl, CF₃, or —O—R¹⁵;
R⁷ is H, C₁-C₈ alkyl, CF₃, or —O—R¹⁶;
R⁸ is H, C₁-C₈ alkyl, CF₃, or —O—R¹⁷;
R⁹ is H, C₁-C₈ alkyl, CF₃, or —O—R¹⁸;
R¹⁰ is H, C₁-C₈ alkyl, CF₃, or —O—R¹⁹;
R¹¹ is H, C₁-C₈ alkyl, CF₃, or —O—R²⁰;
R¹², R¹³, R¹⁴, R¹⁵, R¹⁶, R¹⁷, R¹⁸, R¹⁹ and R²⁰ are each independently C₁-C₄ alkyl, cycloalkyl or benzyl

57. A method of treating motor dysfunction as a complication of other drug treatment, said method comprising administering a pharmaceutically effective amount of AC5 inhibitor to a patient in need of treatment.

58. The method of claim 57, wherein the at least one compound is selected from compounds of formula: 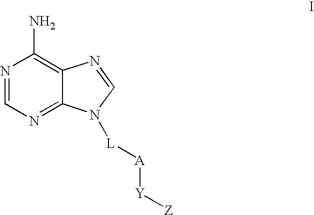
wherein A is a direct link or A is divalent member selected from the group consisting of:
phenyl, thienyl, furanyl, pyrrolyl, indolyl, 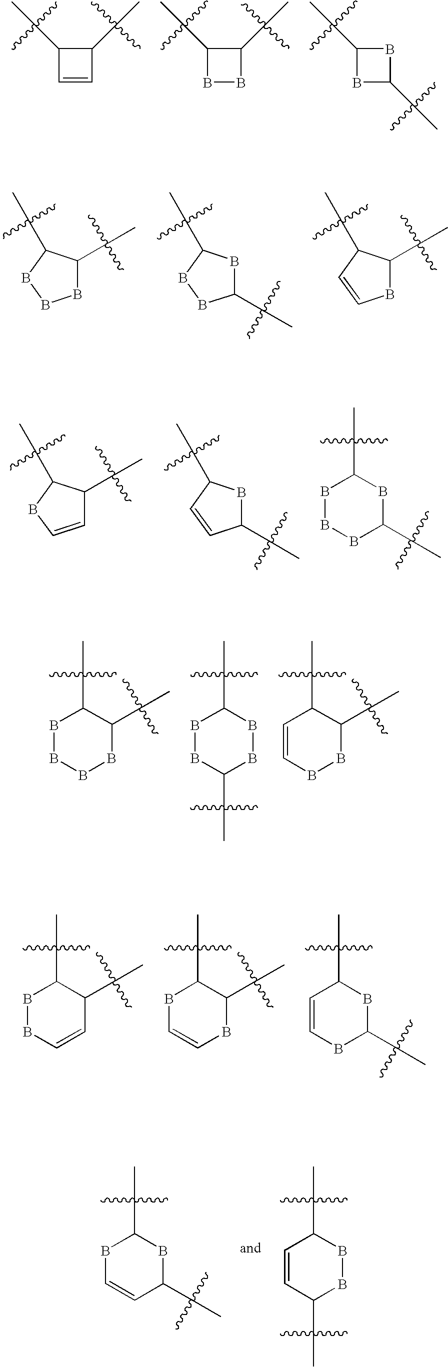
wherein
each B is independently —C(—R¹)(—R²)—, —O— or —N(-J-R³)—, and wherein only one ring B is either O or —N(-J-R³)—;
m and n are each independently an integer from 0-4;
q is an integer from 0 to 8;
Y is —(CH₂)_{q}, —(CH₂)ₘO—, —(CH₂)ₘ—N(-J¹-)-R⁴;
Z is —(CH₂)ₙ—C(═O)—NHOH and —(CH₂)ₙCOOH;
L is —(CH₂)_{q}—, —(CH₂)ₘO—, —(CH₂)ₘ—N-(J²-)-R⁵;
J, J¹ and J² are each independently —C(═O)— or a direct link;
R¹ is H, —N(-J³-R⁶)(-J⁴-R⁷) or —O-J⁵-R⁸;
wherein J³, J⁴ and J⁵ are each independently —C(═O)—, a direct link, or at least one of J³ and J⁴ is a direct link;
R² is H, —N(-J⁶-R⁹)(J⁷-R¹⁰) or —O-J⁸-R¹¹;
wherein J⁶, J⁷ and J⁸ are each independently —C(═O)—, a direct link, or at least one of J⁶ and J⁷ is a direct link;
R³ is H, C₁-C₆ alkyl, CF₃, or —O—R¹²;
R⁴ is H, C₁-C₈ alkyl, CF₃, or —O—R¹³;
R⁵ is H, C₁-C₈ alkyl, CF₃, or —O—R¹⁴;
R⁵ is H, C₁-C₈ alkyl, CF₃, or —O—R¹⁵;
R⁷ is H, C₁-C₈ alkyl, CF₃, or —O—R¹⁶;
R⁸ is H, C₁-C₈ alkyl, CF₃, or —O—R¹⁷;
R⁹ is H, C₁-C₈ alkyl, CF₃, or —O—R¹⁸;
R¹⁰ is H, C₁-C₈ alkyl, CF₃, or —O—R¹⁹;
R¹¹ is H, C₁-C₈ alkyl, CF₃, or —O—R²⁰;
R¹², R¹³, R¹⁴, R¹⁵, R¹⁶, R¹⁷, R¹⁸, R¹⁹ and R²⁰ are each independently C₁-C₄ alkyl, cycloalkyl or benzyl.

59. The method of claim 43 wherein the treatment is for essential tremor.

60. The method of claim 41 wherein the condition or disease is selected from the group consisting of hypertension, abnormal heart rate, and arrhythmia.

61. A pharmaceutical composition according to claim 39 wherein the AC5 regulating compound is present in an amount of about 0.01 to about 0.1 μg/ml.

62. The method of claim 40 wherein the administration is oral.

63. The method of claim 40 further comprising administering the AC5 regulating compound in an amount of about 0.05 to about 500 mg/kg/day.

63. The method of claim 40 further comprising administering the AC5 regulating compound in an amount of about 0.1 to about 100 mg/kg/day.

65. The method of claim 40 further comprising administering the AC5 regulating compound in an amount of about 1.0 to about 50 mg/kg/day.

66. The method of claim 40 wherein the administration is parenteral.

67. The method of claim 40 further comprising administering the AC5 regulating compound in an amount of about 0.01 to about 1000 mg/kg/day.

68. The method of claim 40 further comprising administering the AC5 regulating compound in an amount of about 0.05 to about 500 mg/kg/day.

69. The method of claim 40 further comprising administering the AC5 regulating compound in an amount of about 0.1 to about 100 mg/kg/day.

70. A method for screening of pharmaceutically active treatments of neurodegenerative disorders comprising the administration of treatment to a mouse with the genotype described in Sequence 1.

71. A method for screening of pharmaceutically active treatments of secondary motor dysfunction complications comprising the administration of treatment to a mouse with the genotype described in Sequence 1.

72. A method for screening of pharmaceutically active treatments for prevention of motor dysfunction as a complication of other medical treatment comprising the administration of treatment to a mouse with the genotype described in Sequence 1.

73. A method for screening of pharmaceutically active treatments for hypertension comprising the administration of treatment to a mouse with the genotype described in Sequence 1.

74. recombinant vector comprising the isolated nucleotide Sequence 1.

75. A gene targeting vector comprising the nucleotide Sequence 1 operatively associated with selection marker for neomycin resistance and transfected into a host cell thereby altering the adenylyl cyclase expression in the host.

76. A vector as in claim 35 wherein the host is a mammal.
